(19) United States
(12) Patent Application Publication
(10) Pub. No.: US 2014/0256660 A1
(43) Pub. Date: Sep. 11, 2014

(54) ANTIBIOTIC CONJUGATES LINKED WITH STEROID DRUGS

(71) Applicant: Allergan, Inc., Irvine, CA (US)

(72) Inventors: Santosh C. Sinha, Ladera Ranch, CA (US); Smita S. Bhat, Irvine, CA (US); Ken Chow, Newport Coast, CA (US); LIMING WANG, Irvine, CA (US); MICHAEL E. GARST, Newport Beach, CA (US); MAYSSA ATTAR, Placentia, CA (US); BRANDON D. SWIFT, Camarillo, CA (US)

(73) Assignee: Allergan, Inc., Irvine, CA (US)

(21) Appl. No.: 14/199,101

(22) Filed: Mar. 6, 2014

Related U.S. Application Data
(60) Provisional application No. 61/775,121, filed on Mar. 8, 2013.

(51) Int. Cl. A61K 47/48; A61K 9/00

(52) U.S. Cl. CPC A61K 47/48115; A61K 9/0048
USPC 514/26; 540/110; 514/176; 536/5; 540/120; 514/179

(57) ABSTRACT

The present invention describes novel single drug entities, formed by the linkage of an antibiotic with a steroidal drug via a linker. Upon topical application to the eye, the conjugate would undergo enzymatic and/or hydrolytic cleavage to release the individual drugs.

### RELATED APPLICATIONS

This application claims the benefit of U.S. Provisional Patent Application Ser. No. 61/775,121 filed Mar. 8, 2013, the disclosure of which is hereby incorporated in its entirety by reference.

### FIELD OF THE INVENTION

The present invention describes novel single drug entities, formed by the linkage of an antibiotic with a steroidal drug via a linker. Upon topical application to the eye, the conjugate would undergo enzymatic and/or hydrolytic cleavage to release the individual drugs.

### SUMMARY OF THE INVENTION

Due to increasing bacterial resistance to antibiotics there is a constant need for antibiotic compounds. A conjugate drug, also referred to as a co-drug, a pro-drug, or a hybrid drug, comprises two or more different or same drugs within one single chemical entity wherein each drug contains an appropriate chemical functionality to enable them to be connected together, by means of a covalent linker, which is cleavable and biologically labile.

Hybrid drugs may incorporate at least two drugs joined together by a linker moiety such as an ester, a carboxylate, a carbonyl, a carbonate, an amido, a carbamate, a ketone, an amino, an oxo, an ethylene glycol, an alkylene, a polyethylene glycol, which is cleaved enzymatically or hydrolytically in vivo to release the active drugs.

By appropriate structural design of these linkers, it may be possible to control the release of each individual drug. When the drugs are chemically combined, the resulting hybrid drug will usually have different physicochemical properties compared to the individual parent drugs, which may provide superior properties for delivery when compared to delivery of a physical mixture of the drugs. The antibiotic moiety and the steroid moiety, of the compounds disclosed herein are connected each separately via a covalent bond to a linker such that said compound degrades in vivo to yield the individual antibiotic and steroid.

Degradation of these covalent bonds generally, yields the corresponding carboxylic acid, or alcohol, or amine by hydrolysis or by a related reaction. A compound which degrades in vivo to yield the individual antibiotic and steroid, produces the active antibiotic drug and the steroid drug at some point in the metabolic process of the claimed compound. The linker structure may be designed such that cleavage at a single covalent bond initiates a cascade of reactions that results in the ultimate release of the active drugs.

### BRIEF DESCRIPTION OF THE DRAWINGS

FIG. 1 Shows the cellular uptake of ester linked hybrid (parent) compounds and the hydrolyzed metabolites (steroid and antibiotic) after a two hour incubation with Human Corneal Epithelial Cells.

FIG. 2 Shows the mean±standard error of the enzymatically cleaved steroid area under the concentration-time profile (AUC₀₋₁₀ₕᵣ) Following a Single Topical Ocular Dose 10 hr, of 0.4% of the Hybrid Compound in Rabbits.

FIG. 3 Shows the mean±standard error of the enzymatically cleaved antibiotic area under the concentration-time profile (AUC₀₋₁₀ₕᵣ) Following a Single Topical Ocular Dose of 0.4% of the Hybrid Compound in Rabbits

### DETAILED DESCRIPTION OF THE INVENTION

The hybrid drugs of the invention provide a unique delivery of an antibiotic and a steroid for the treatment and prevention of ophthalmic bacterial infections and anti-inflammatory conditions. A single drug entity is advantageous to individual dosing of each drug because of the ability for simultaneous dosing and elimination of washout concerns when applying each drug separately.

The hybrid drugs of the invention have anti-bacterial activity and anti-inflammatory activity and are very useful compounds capable of producing the effect of an antibacterial drug and an anti-inflammatory drug with a broad range of activity in monotherapy.

The use of an antibiotic/steroid hybrid drug is indicated where the risk of infection is high or where there is an expectation that potentially dangerous numbers of bacteria will be present in the eye. The anti-inflammatory component of the composition is useful in treating inflammation associated with physical trauma to ophthalmic tissues, inflammation associated with bacterial infections and inflammation resulting from surgical procedures. The combination of an antibiotic and steroid is also useful in post-operative inflammation where there is an increased chance of bacterial infection. The composition of the invention may also be used prophylactically in connection with various ophthalmic surgical procedures that create a risk of bacterial infection. Other examples of ophthalmic conditions which may be treated with the compositions of the present invention include infective conditions with associated inflammation and where the use of steroids is acceptable; such conditions may include, but not limited toconjunctivitis, keratitis, blepharitis, endophthalmitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, red eye, hyperemia, post-surgical inflammation, inflammatory conditions of the palpebral and bulbar conjunctiva, cornea, and anterior segment of the globe, such as allergic conjunctivitis, ocular rosacea, dry eye, blepharitis, endophthalmitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

The present invention relates to hybrid drugs comprising at one antibiotic moiety and one steroid moiety, or a pharmaceutical salt thereof, which are separately connected via a covalent bond to a linker such that said covalent bonds degrade in vivo to yield the respective antibiotic and steroid independently.

In another aspect, the present invention relates to hybrid drugs, which degrade in vivo into an antibiotic and a steroidal drug.

In another aspect, the present invention relates to hybrid drugs having two bonds, wherein said bonds are asymmetrically degraded in vivo to release the two independent drugs: an antibiotic and a steroidal drug.

The hybrid drugs disclosed herein comprise antibiotics moieties belonging to distinct classes: fluoroquinolones, cephalosporins, chloramphenicol, aminoglycosides, penicillins, erythromycin, macrolide antibiotics and oxazolidionones.

Fluoroquinolones include, but are not limited to: levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, and sitafloxacin.

Cephalosporins include, but are not limited to: loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, and cephalothin.

Aminoglycosides include, but are not limited to: tobramycin, streptomycin, gentamicin, kanamycin, amikacin and netilmicin.

Penicillins include, but are not limited to: penicillin G, ticarcillin, methicillin, phenthicillin, cloxacillin, dicloxacillin, nafcillin, oxacillin.

Macrolide antibiotics include, but are not limited to: erythromycin and azithromycin.

Oxazolidinones include, but are not limited to: linezolid.

In another embodiment the compounds disclosed herein comprise one antibiotic drug moiety selected from levofloxacin, moxifloxacin, gatifloxacin, gemifloxacin, besifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin, oxacillin, erythromycin and azithromycin.

In another embodiment the hybrid compounds disclosed herein comprise a steroidal moiety selected from: dexmethasone, betamethasone, triamcinolone acetonide, prednisolone and hydrocortisone.

In another embodiment the hybrid compounds disclosed herein comprise a antibiotic moiety is selected from: moxifloxacin, besifloxacin, gatifloxacin, amikacin chloramphenicol, tobramycin and clindamycin.

In another embodiment the hybrid compounds disclosed herein comprise a gatifloxacin moiety and the steroid moiety is selected from: dexmethasone, betamethasone, triamcinolone acetonide, prednisolone and hydrocortisone.

In another embodiment the hybrid compounds disclosed herein comprise a moxifloxacin moiety and the steroid moiety is selected from: dexmethasone, betamethasone, triamcinolone acetonide, prednisolone and hydrocortisone.

In another embodiment the compounds disclosed herein comprise one linker and one antibiotic moiety selected from levofloxacin, moxifloxacin, gatifloxacin, clindamycin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin.

In another embodiment the hybrid compounds disclosed herein comprise one linker and a pro-drug moiety and one antibiotic moiety selected from levofloxacin, clindamycin, besifloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin.

In another embodiment the hybrid compounds disclosed herein comprise one linker and a pro-drug moiety and one antibiotic moiety selected from levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin; and one steroid moiety selected from: dexmethasone, betamethasone, triamcinolone acetonide, prednisolone and hydrocortisone.

In another embodiment the compounds disclosed herein comprise one linker and one antibiotic moiety selected from levofloxacin, moxifloxacin, gatifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin and oxacillin; and one steroid moiety selected from: dexmethasone, betamethasone, triamcinolone acetonide, prednisolone and hydrocortisone.

In another aspect the invention provides a method comprising administrating to an eye of a mammal a pharmaceutical composition comprising a therapeutically active amount of a hybrid drug comprising one antibiotic moieties and one steroid moiety, which are connected via two separate covalent bonds to a linker such that said covalent bonds degrade in vivo to yield the antibiotic and the steroid, wherein each bond is an ester bond or an amide bond, wherein said method is effective in the treatment of a bacterial infection or an inflammation affecting said eye.

In another aspect the invention provides a method according, wherein the bacterial infection is selected from: conjunctivitis, keratitis, blepharitis, dacyrocystitis, hordeolum, corneal ulcers, anterior blepharitis, posterior blepharitis, endophthalmitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies and allergy.

In another aspect the invention provides a method comprising administrating to an eye of a human a pharmaceutical composition comprising a therapeutically active amount of a hybrid drug comprising one antibiotic moieties and one steroid moiety, which are connected via two separate covalent bonds to a linker such that said covalent bonds degrade in vivo to yield the antibiotic and the steroid, wherein each bond is an ester bond or an amide bond, wherein said method is effective in the treatment of a bacterial infection or an inflammation affecting said eye.

In another aspect the invention provides a pharmaceutical composition comprising a hybrid drug comprising an antibiotic moiety and a steroid, which are connected via two separate covalent bonds to a linker such that said covalent bonds degrade in vivo to yield the antibiotic moiety and the steroid moiety, and wherein each bond is an ester bond or an amide bond, and wherein said pharmaceutical composition is formulated for topical ophthalmic administration.

Depending of the bond formation site, the antibiotic moiety can be linked via an ester bond or via an amido bond and the steroid moiety can be linked via an ester bond, as shown in the following schemes:

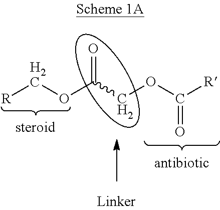

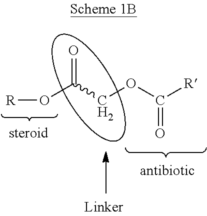

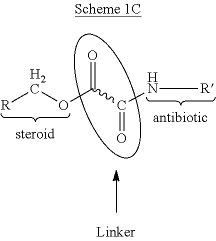

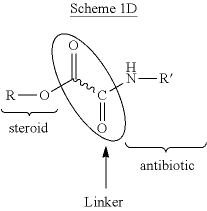

In another aspect the invention provides compounds which may comprise a linker moiety selected from, but not limited to, an ester, a carboxylate, a carbonyl, a carbonate, an amido, a carbamate, a ketone, an amino, an oxo, an ethylene glycol, a polyethylene glycol, an ethylene.

In another aspect, the invention provides compounds which may comprise a linker moiety comprising any combination of an ester, a carboxylate, a carbonyl, a carbonate, an amido, a carbamate, a ketone, an ethylene, an amino, an oxo, an ethylene glycol and/or a polyethylene glycol. Such linkers moieties and linker structures are exemplified in Table 1.

Examples of ester moieties comprised in the linkers are:

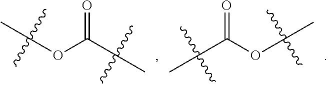 [SMILES: *OC(*)=O ; *OC(*)=O]

Examples of carboxylate moieties comprised in the linkers are:

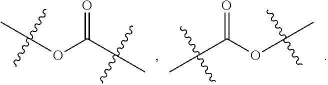 [SMILES: *OC(*)=O ; *OC(*)=O]

Example of a carbonyl moiety comprised in the linkers is

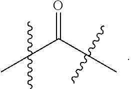 [SMILES: *C(*)=O]

Example of a carbonate moiety comprised in the linkers is:

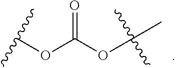 [SMILES: *OC(=O)O*]

Examples of amido moieties comprised in the linkers are:

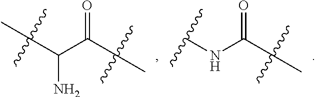 [SMILES: *C(=O)C(*)N ; *NC(*)=O]

Example of carbamate moiety comprised in the linkers is:

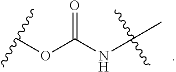 [SMILES: *NC(=O)O*]

Example of a ketone moiety comprised in the linkers is:

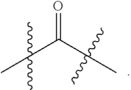 [SMILES: *C(*)=O]

Examples of amino moieties comprised in the linkers are:

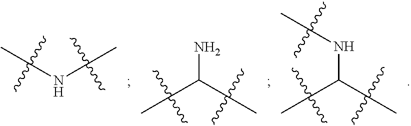

Example of an oxo moiety comprised in the linker is:

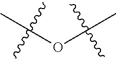 [SMILES: *O*]

Example of ethylene glycol moieties comprised in the linkers are:

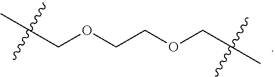 [SMILES: *COCCOC*]

Example of polyethylene glycol moiety comprised in the linkers is:

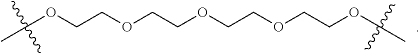 [SMILES: *OCCOCCOCCOCCO*]

Further the compounds disclosed herein comprise a linker selected from Table 1:

TABLE 1

| | Linker Number | | | | |
|---|---|---|---|---|---|
| Linker Structure | | n = 0 | n = 1 | n = 2 | n = 3 |
| 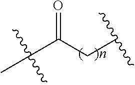 | | | L1 | L2 | L3 |
| 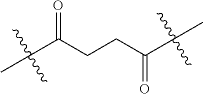 [SMILES: *C(=O)CCC(*)=O] | L4 | | | | |
| 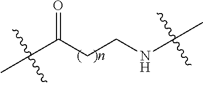 | | | L5 | L6 | |
| 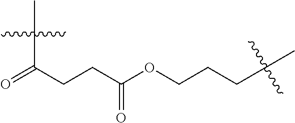 [SMILES: *CCCOC(=O)CCC(*)=O] | L7 | | | | |
| 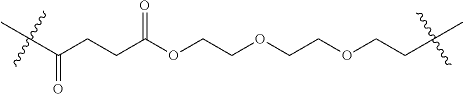 [SMILES: *CCOCCOCCOC(=O)CCC(*)=O] | L8 | | | | |
| 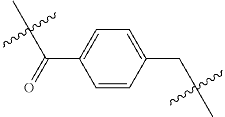 [SMILES: *Cc1ccc(C(*)=O)cc1] | L9 | | | | |
| 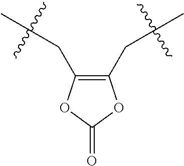 [SMILES: *Cc1oc(=O)oc1C*] | L10 | | | | |
| 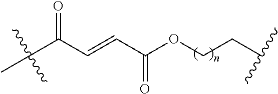 | | | L11 | L27 | |
| 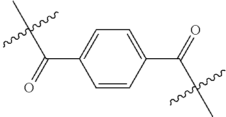 [SMILES: *C(=O)c1ccc(C(*)=O)cc1] | L12 | | | | |
| 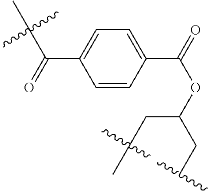 [SMILES: *CC(C*)OC(=O)c1ccc(C(*)=O)cc1] | L13 | | | | |
| 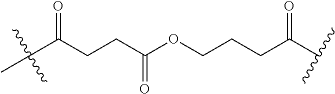 [SMILES: *C(=O)CCCOC(=O)CCC(*)=O] | L14 | | | | |
| 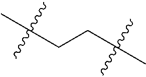 [SMILES: *CC*] | L15 | | | | |
| 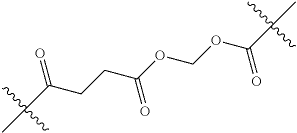 [SMILES: *C(=O)CCC(=O)OCOC(*)=O] | L16 | | | | |
| 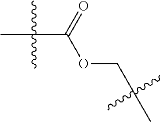 [SMILES: *COC(*)=O] | L17 | | | | |
| 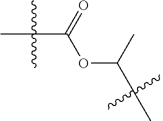 [SMILES: *C(=O)OC(*)C] | L18 | | | | |
| 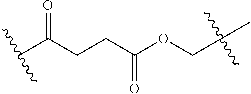 [SMILES: *COC(=O)CCC(*)=O] | L19 | | | | |
| 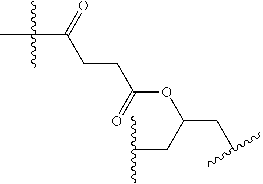 [SMILES: *CC(C*)OC(=O)CCC(*)=O] | L20 | | | | |
| 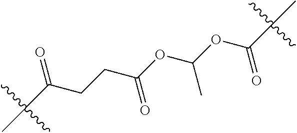 [SMILES: *C(=O)CCC(=O)OC(C)OC(*)=O] | L21 | | | | |
| 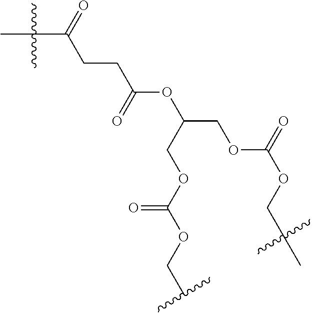 [SMILES: *COC(=O)OCC(COC(=O)OC*)OC(=O)CCC(*)=O] | L22 | | | | |
| 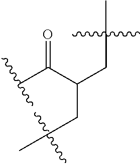 [SMILES: *CC(C*)C(*)=O] | L23 | | | | |
| 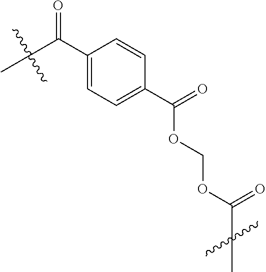 [SMILES: *C(=O)OCOC(=O)c1ccc(C(*)=O)cc1] | L24 | | | | |
| 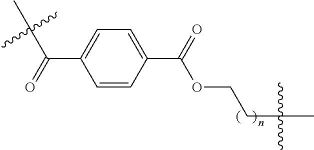 | | L28 | L25 | L26 | |
| 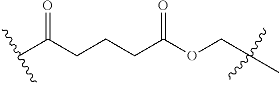 [SMILES: *COC(=O)CCCC(*)=O] | L29 | | | | |
| 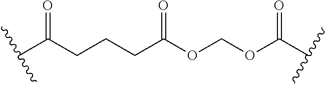 [SMILES: *C(=O)CCCC(=O)OCOC(*)=O] | L30 | | | | |
| 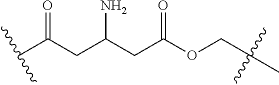 [SMILES: *COC(=O)CC(N)CC(*)=O] | L31 | | | | |
| 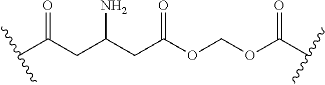 [SMILES: *C(=O)CC(N)CC(=O)OCOC(*)=O] | L32 | | | | |
| 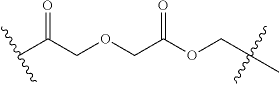 [SMILES: *COC(=O)COCC(*)=O] | L33 | | | | |
| 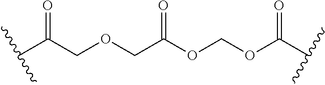 [SMILES: *C(=O)COCC(=O)OCOC(*)=O] | L34 | | | | |
| 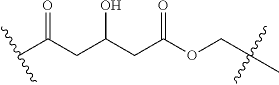 [SMILES: *COC(=O)CC(O)CC(*)=O] | L35 | | | | |
| 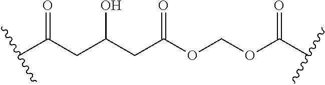 [SMILES: *C(=O)CC(O)CC(=O)OCOC(*)=O] | L36 | | | | |
| 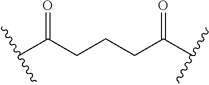 [SMILES: *C(=O)CCCC(*)=O] | L37 | | | | |
| 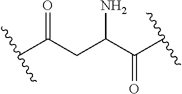 [SMILES: *C(=O)CC(N)C(*)=O] | L38 | | | | |
| 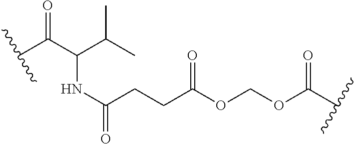 [SMILES: *C(=O)OCOC(=O)CCC(=O)NC(C(*)=O)C(C)C] | L39 | | | | |
| 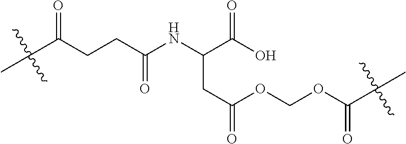 [SMILES: *C(=O)CCC(=O)NC(CC(=O)OCOC(*)=O)C(=O)O] | L40 | | | | |
| 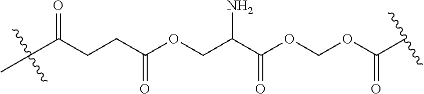 [SMILES: *C(=O)CCC(=O)OCC(N)C(=O)OCOC(*)=O] | L41 | | | | |
| 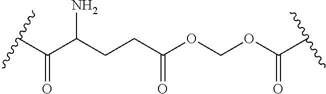 [SMILES: *C(=O)OCOC(=O)CCC(N)C(*)=O] | L42 | | | | |
| 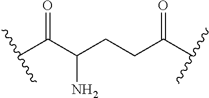 [SMILES: *C(=O)CCC(N)C(*)=O] | L43 | | | | |
| 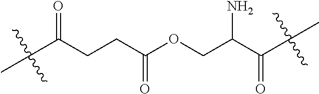 [SMILES: *C(=O)CCC(=O)OCC(N)C(*)=O] | L44 | | | | |
| 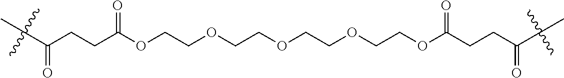 [SMILES: *C(=O)CCC(=O)OCCOCCOCCOCCOC(=O)CCC(*)=O] | L45 | | | | |
| 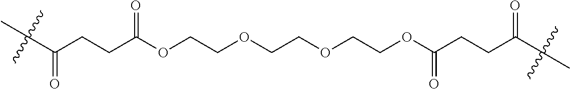 [SMILES: *C(=O)CCC(=O)OCCOCCOCCOC(=O)CCC(*)=O] | L46 | | | | |
| 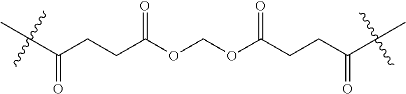 [SMILES: *C(=O)CCC(=O)OCOC(=O)CCC(*)=O] | L47 | | | | |
| 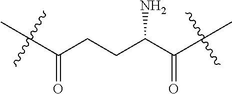 [SMILES: *C(=O)CC[C@H](N)C(*)=O] | L48 | | | | |
| 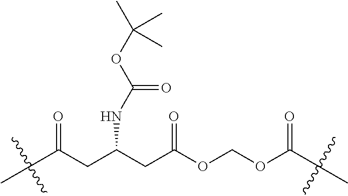 [SMILES: *C(=O)C[C@@H](CC(=O)OCOC(*)=O)NC(=O)OC(C)(C)C] | L49 | | | | |
| 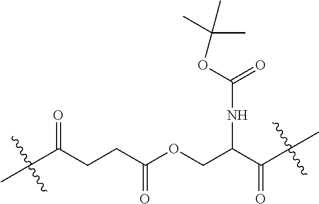 [SMILES: *C(=O)CCC(=O)OCC(NC(=O)OC(C)(C)C)C(*)=O] | L50 | | | | |
| 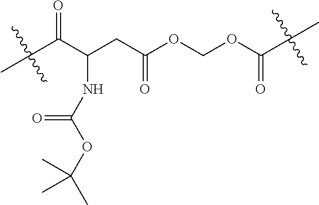 [SMILES: *C(=O)OCOC(=O)CC(NC(=O)OC(C)(C)C)C(*)=O] | L51 | | | | |
| 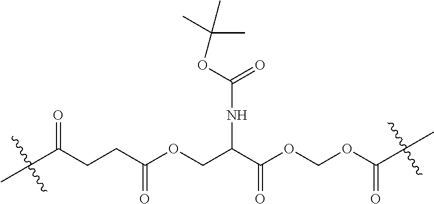 [SMILES: *C(=O)CCC(=O)OCC(NC(=O)OC(C)(C)C)C(=O)OCOC(*)=O] | L52 | | | | |
| 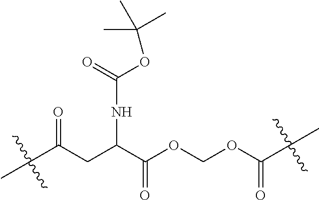 [SMILES: *C(=O)CC(NC(=O)OC(C)(C)C)C(=O)OCOC(*)=O] | L53 | | | | |
| 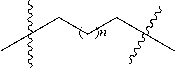 | | L54 | L55 | | |
| 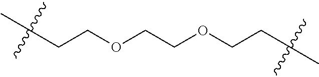 [SMILES: *CCOCCOCC*] | L56 | | | | |
| 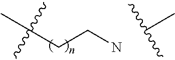 | | | L57 | | |
| 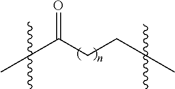 | | L58 | | L59 | |
| 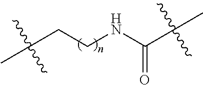 | | | L60 | | |
| 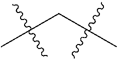 [SMILES: *C*] | L61 | | | | |
| 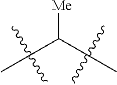 [SMILES: *C(*)C] | L62 | | | | |
| 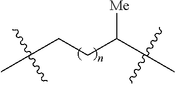 | | | L63 | | |
| 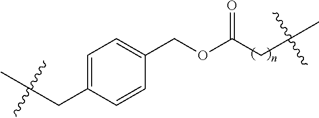 | L64 | | | | |
| 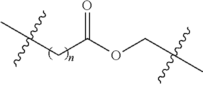 | | L66 | L65 | | |
| 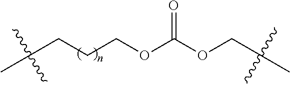 | | | L67 | | |
| 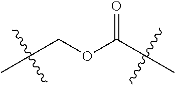 [SMILES: *COC(*)=O] | L68 | | | | |
| 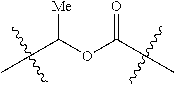 [SMILES: *C(=O)OC(*)C] | L69 | | | | |
| 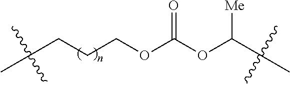 | | | L70 | | |
| 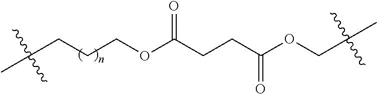 | | | L71 | | |
| 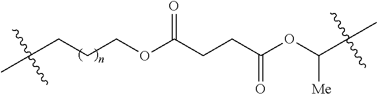 | | | L72 | | |
| 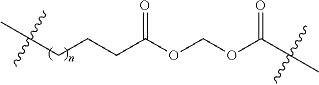 | | | L73 | | |
| 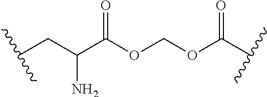 [SMILES: *CC(N)C(=O)OCOC(*)=O] | L74 | | | | |
| 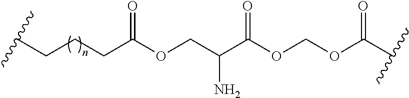 | | | L75 | | |
| 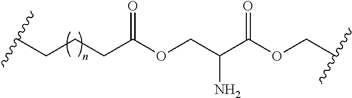 | | | L76 | | |
| 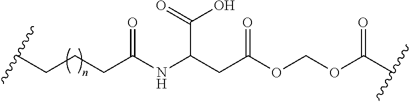 | | | L77 | | |
| 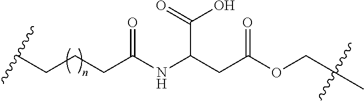 | | | L78 | | |
| 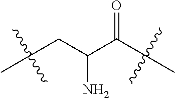 [SMILES: *CC(N)C(*)=O] | L79 | | | | |
| 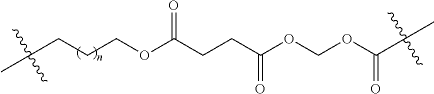 | | | L80 | | |
| 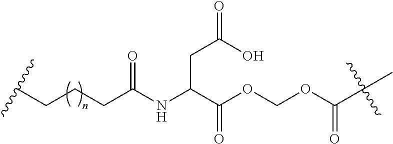 | | | L81 | | |
| 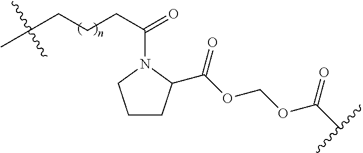 | | | L82 | | |
| 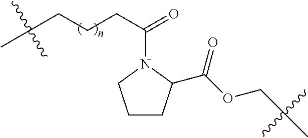 | | | L83 | | |
| 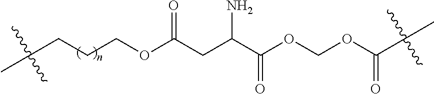 | | | L84 | | |
| 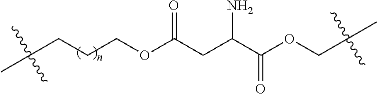 | | | L88 | | |
| 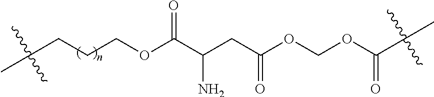 | | | L89 | | |
| 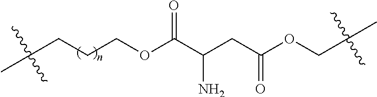 | | | L90 | | |
| 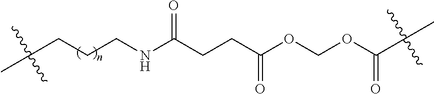 | | | L91 | | |
| 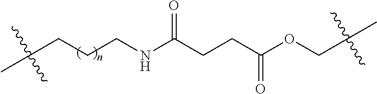 | | | L92 | | |
| 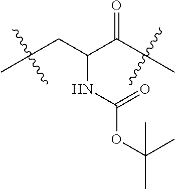 [SMILES: *CC(NC(=O)OC(C)(C)C)C(*)=O] | L93 | | | | |
| 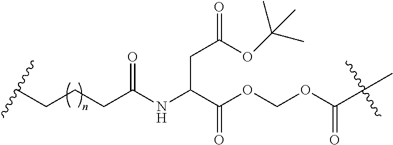 | | | L94 | | |
| 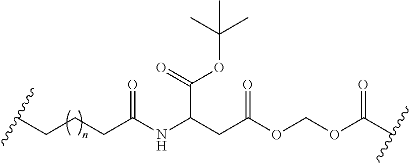 | | | L95 | | |
| 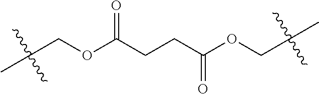 [SMILES: *COC(=O)CCC(=O)OC*] | L96 | | | | |
| 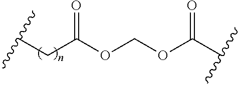 | | | L97 | | |
| 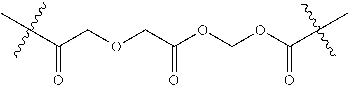 [SMILES: *C(=O)COCC(=O)OCOC(*)=O] | L98 | | | | |
| 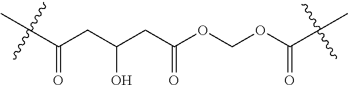 [SMILES: *C(=O)CC(O)CC(=O)OCOC(*)=O] | L99 | | | | |
| 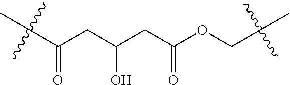 [SMILES: *COC(=O)CC(O)CC(*)=O] | L100 | | | | |
| 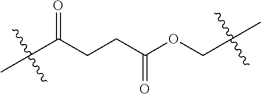 [SMILES: *COC(=O)CCC(*)=O] | L101 | | | | |
| 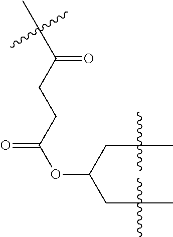 [SMILES: *CC(C*)OC(=O)CCC(*)=O] | L102 | | | | |
| 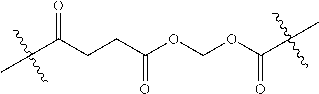 [SMILES: *C(=O)CCC(=O)OCOC(*)=O] | L103 | | | | |
| 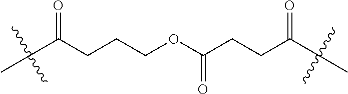 [SMILES: *C(=O)CCCOC(=O)CCC(*)=O] | L104 | | | | |
| 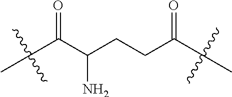 [SMILES: *C(=O)CCC(N)C(*)=O] | L105 | | | | |
| 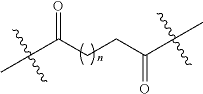 | | | L107 | | |
| 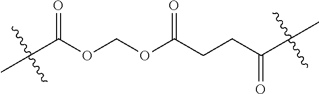 [SMILES: *C(=O)CCC(=O)OCOC(*)=O] | L108 | | | | |
| 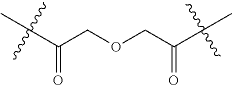 [SMILES: *C(=O)COCC(*)=O] | L109 | | | | |
| 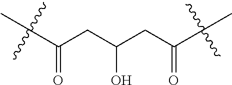 [SMILES: *C(=O)CC(O)CC(*)=O] | L110 | | | | |
| 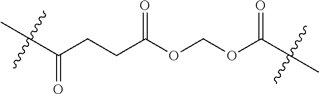 [SMILES: *C(=O)CCC(=O)OCOC(*)=O] | L111 | | | | |
| 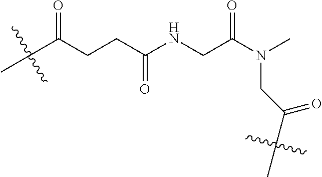 [SMILES: *C(=O)CCC(=O)NCC(=O)N(C)CC(*)=O] | L112 | | | | |
| 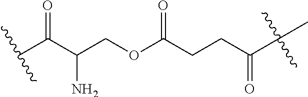 [SMILES: *C(=O)CCC(=O)OCC(N)C(*)=O] | L113 | | | | |
| 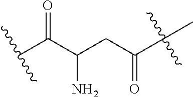 [SMILES: *C(=O)CC(N)C(*)=O] | L114 | | | | |
| 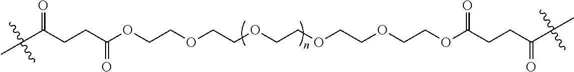 | | | L115 | L129 | L106 |
| 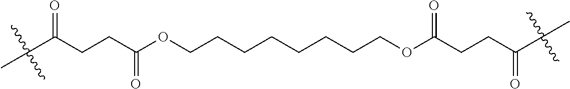 [SMILES: *C(=O)CCC(=O)OCCCCCCCCOC(=O)CCC(*)=O] | L116 | | | | |
| 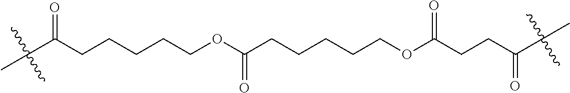 [SMILES: *C(=O)CCCCCOC(=O)CCCCCOC(=O)CCC(*)=O] | L117 | | | | |
| 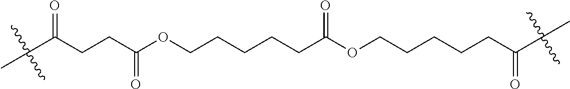 [SMILES: *C(=O)CCCCCOC(=O)CCCCCOC(=O)CCC(*)=O] | L118 | | | | |
| 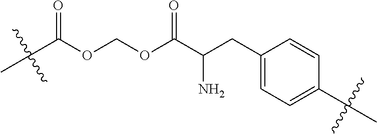 [SMILES: *C(=O)OCOC(=O)C(N)Cc1ccc(*)cc1] | L119 | | | | |
| 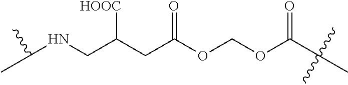 | L120 | | | | |
| 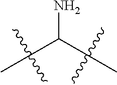 [SMILES: *C(*)N] | L121 | | | | |
| 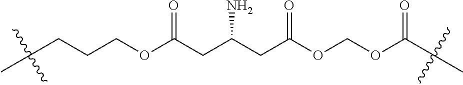 [SMILES: *CCCOC(=O)C[C@H](N)CC(=O)OCOC(*)=O] | L122 | | | | |
| 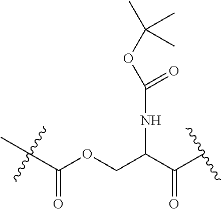 [SMILES: *C(=O)OCC(NC(=O)OC(C)(C)C)C(*)=O] | L123 | | | | |
| 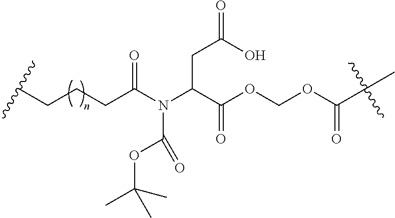 | L124 | | | | |
| 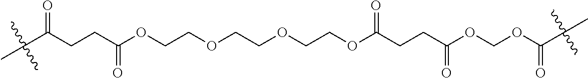 [SMILES: *C(=O)CCC(=O)OCCOCCOCCOC(=O)CCC(=O)OCOC(*)=O] | L125 | | | | |
| 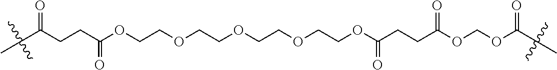 [SMILES: *C(=O)CCC(=O)OCCOCCOCCOCCOC(=O)CCC(=O)OCOC(*)=O] | L126 | | | | |
| 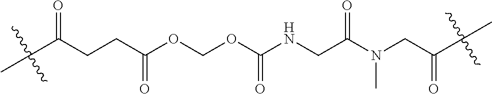 [SMILES: *C(=O)CCC(=O)OCOC(=O)NCC(=O)N(C)CC(*)=O] | L127 | | | | |
| 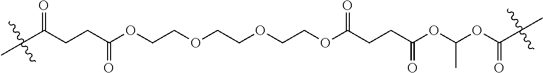 [SMILES: *C(=O)CCC(=O)OCCOCCOCCOC(=O)CCC(=O)OC(C)OC(*)=O] | L128 | | | | |

Further, the hybrid drugs of the invention may comprise a pro-drug moiety as described below in Table 2:

TABLE 2

| | Pro-drug |
|---|---|
| | Moiety |
| Pro-drug Moiety Structure | Number |
| 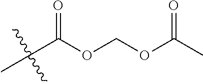 [SMILES: *C(=O)OCOC(C)=O] | P1 |
| 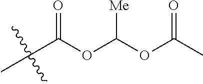 [SMILES: *C(=O)OC(C)OC(C)=O] | P2 |
| 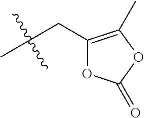 [SMILES: *Cc1oc(=O)oc1C] | P3 |
| 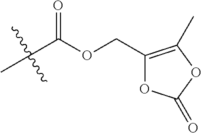 [SMILES: *C(=O)OCc1oc(=O)oc1C] | P4 |
| 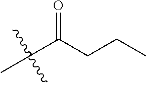 [SMILES: *C(=O)CCC] | P5 |
| 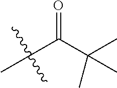 [SMILES: *C(=O)C(C)(C)C] | P6 |
| 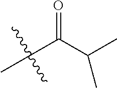 [SMILES: *C(=O)C(C)C] | P7 |
| 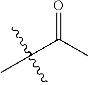 [SMILES: *C(C)=O] | P8 |
| 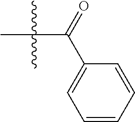 [SMILES: *C(=O)c1ccccc1] | P9 |
| 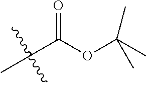 [SMILES: *C(=O)OC(C)(C)C] | P10 |
| 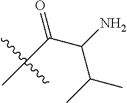 [SMILES: *C(=O)C(N)C(C)C] | P11 |
| 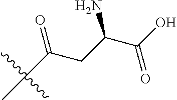 [SMILES: *C(=O)C[C@@H](N)C(=O)O] | P12 |
| 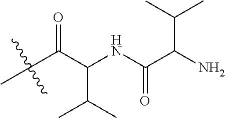 [SMILES: *C(=O)C(NC(=O)C(N)C(C)C)C(C)C] | P13 |
| 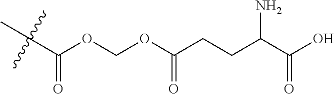 [SMILES: *C(=O)OCOC(=O)CCC(N)C(=O)O] | P14 |
| 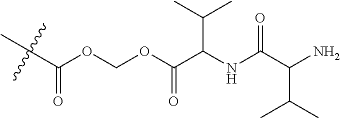 [SMILES: *C(=O)OCOC(=O)C(NC(=O)C(N)C(C)C)C(C)C] | P15 |

Compounds of the invention are shown in Table 3:

TABLE 3

| Compound No | IUPAC Name |
|---|---|
| 39 | rel-1-cyclopropyl-7-[4-(5-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-5-oxopentanoyl)- |
| | 3-methylpiperazin-1-yl]-6-fluoro-8-methoxy-4-oxo-1,4- |
| | dihydroquinoline-3-carboxylic acid |
| 38 | rel-7-[4-({[(4-amino-5-{2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethoxy}-5- |
| | oxopentanoyl)oxy]methoxy}carbonyl)-3-methylpiperazin-1-yl]-1- |
| | cyclopropyl-6-fluoro-8-methoxy-4-oxo-1,4-dihydroquinoline-3- |
| | carboxylic acid |
| 37 | rel-7-{4-[(4R)-4-amino-5-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-5-oxopentanoyl]- |
| | 3-methylpiperazin-1-yl}-1-cyclopropyl-6-fluoro-8-methoxy-4-oxo-1,4- |
| | dihydroquinoline-3-carboxylic acid |
| 36 | rel-7-{4-[(2R)-2-amino-4-{2-[(10S,11R,13R,17S)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-4-oxobutanoyl]-3- |
| | methylpiperazin-1-yl}-1-cyclopropyl-6-fluoro-8-methoxy-4-oxo-1,4- |
| | dihydroquinoline-3-carboxylic acid |
| 35 | rel-7-{4-[({[(3R)-3-amino-5-{2-[(10S,11R,13R,17S)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-5- |
| | oxopentanoyl]oxy}methoxy)carbonyl]-3-methylpiperazin-1-yl}-1- |
| | cyclopropyl-6-fluoro-8-methoxy-4-oxo-1,4-dihydroquinoline-3- |
| | carboxylic acid |
| 46 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-pentanedioate |
| 14 | rel-7-(4-{7-carboxy-15-[(9R,10S,11S,13S,16R,17R)-9-fluoro-11,17- |
| | dihydroxy-10,13,16-trimethyl-3-oxo-6,7,8,9,10,11,12,13,14,15,16,17- |
| | dodecahydro-3H-cyclopenta[a]phenanthren-17-yl]-5,9,12,15- |
| | tetraoxo-2,4,13-trioxa-8-azapentadecan-1-oyl}-3-methylpiperazin-1- |
| | yl)-1-cyclopropyl-6-fluoro-8-methoxy-4-oxo-1,4-dihydroquinoline-3- |
| | carboxylic acid |
| 34 | rel-7-(4-{2-amino-3-[(4-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-4- |
| | oxobutanoyl)oxy]propanoyl}-3-methylpiperazin-1-yl)-1-cyclopropyl-6- |
| | fluoro-8-methoxy-4-oxo-1,4-dihydroquinoline-3-carboxylic acid |
| 33 | rel-1-cyclopropyl-7-[4-({[(5-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-5- |
| | oxopentanoyl)oxy]methoxy}carbonyl)-3-methylpiperazin-1-yl]-6- |
| | fluoro-8-methoxy-4-oxo-1,4-dihydroquinoline-3-carboxylic acid |
| 32 | rel-7-[4-({[(3-amino-4-{2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethoxy}-4- |
| | oxobutanoyl)oxy]methoxy}carbonyl)-3-methylpiperazin-1-yl]-1- |
| | cyclopropyl-6-fluoro-8-methoxy-4-oxo-1,4-dihydroquinoline-3- |
| | carboxylic acid |
| 31 | rel-7-(4-{6-amino-15-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-5,9,12,15-tetraoxo-2,4,8,13- |
| | tetraoxapentadecan-1-oyl}-3-methylpiperazin-1-yl)-1-cyclopropyl-6- |
| | fluoro-8-methoxy-4-oxo-1,4-dihydroquinoline-3-carboxylic acid |
| 30 | rel-7-(4-{7-carboxy-15-[(10R,11S,13S,17R)-11,17-dihydroxy-10,13- |
| | dimethyl-3-oxo-6,7,8,9,10,11,12,13,14, 15,16,17-dodecahydro-3H- |
| | cyclopenta[a]phenanthren-17-yl]-5,9,12,15-tetraoxo-2,4,13-trioxa-8- |
| | azapentadecan-1-oyl}-3-methylpiperazin-1-yl)-1-cyclopropyl-6-fluoro- |
| | 8-methoxy-4-oxo-1,4-dihydroquinoline-3-carboxylic acid |
| 53 | rel-1-cyclopropyl-6-fluoro-7-{1-[(10R)-14- |
| | [(9S,10R,11R,13R,16S,17S)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-5,8,11,14-tetraoxo-10-(propan-2-yl)- |
| | 2,4,12-trioxa-9-azatetradecan-1-oyl]octahydro-6H-pyrrolo[3,4- |
| | b]pyridin-6-yl}-8-methoxy-4-oxo-1,4-dihydroquinoline-3-carboxylic |
| | acid |
| 13 | rel-1-cyclopropyl-6-fluoro-7-{4-[(10R)-14- |
| | [(9S,10R,11R,13R,16S,17S)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-5,8,11,14-tetraoxo-10-(propan-2-yl)- |
| | 2,4,12-trioxa-9-azatetradecan-1-oyl]-3-methylpiperazin-1-yl}-8- |
| | methoxy-4-oxo-1,4-dihydroquinoline-3-carboxylic acid |
| 61 | rel-1-cyclopropyl-7-{1-[(10R)-14-[(10S,11R,13R,17S)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-5,8,11,14- |
| | tetraoxo-10-(propan-2-yl)-2,4,12-trioxa-9-azatetradecan-1- |
| | oyl]octahydro-6H-pyrrolo[3,4-b]pyridin-6-yl}-6-fluoro-8-methoxy-4- |
| | oxo-1,4-dihydroquinoline-3-carboxylic acid |
| 29 | rel-1-cyclopropyl-7-{4-[(10R)-14-[(10S,11R,13R,17S)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-5,8,11,14- |
| | tetraoxo-10-(propan-2-yl)-2,4,12-trioxa-9-azatetradecan-1-oyl]-3- |
| | methylpiperazin-1-yl}-6-fluoro-8-methoxy-4-oxo-1,4-dihydroquinoline- |
| | 3-carboxylic acid |
| 28 | rel-7-[4-({[(2-amino-4-{2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethoxy}-4- |
| | oxobutanoyl)oxy]methoxy}carbonyl)-3-methylpiperazin-1-yl]-1- |
| | cyclopropyl-6-fluoro-8-methoxy-4-oxo-1,4-dihydroquinoline-3- |
| | carboxylic acid |
| 81 | [({[(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methoxy}carbonyl)oxy]methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 70 | [({7-[(3R)-3-aminoazepan-1-yl]-8-chloro-1-cyclopropyl-6-fluoro-4-oxo- |
| | 1,4-dihydroquinolin-3-yl}carbonyl)oxy]methyl 2- |
| | [(9R,10S,11S,13S,16R,17R)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-2-oxoethyl rel-butanedioate |
| 72 | [({7-[(3R)-3-aminoazepan-1-yl]-8-chloro-1-cyclopropyl-6-fluoro-4-oxo- |
| | 1,4-dihydroquinolin-3-yl}carbonyl)oxy]methyl 2-[(10R,11S,13S,17R)- |
| | 11,17-dihydroxy-10,13-dimethyl-3-oxo- |
| | 2,3,6,7,8,9,10,11,12,13,14,15,16,17-tetradecahydro-1H- |
| | cyclopenta[a]phenanthren-17-yl]-2-oxoethyl rel-butanedioate |
| 67 | ({[1-cyclopropyl-6-fluoro-8-methoxy-7-(octahydro-6H-pyrrolo[3,4- |
| | b]pyridin-6-yl)-4-oxo-1,4-dihydroquinolin-3-yl]carbonyl}oxy)methyl 2- |
| | [(10R,11S,13S,17R)-11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-benzene-1,4- |
| | dicarboxylate |
| 45 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-benzene-1,4- |
| | dicarboxylate |
| 58 | ({[1-cyclopropyl-6-fluoro-8-methoxy-7-(octahydro-6H-pyrrolo[3,4- |
| | b]pyridin-6-yl)-4-oxo-1,4-dihydroquinolin-3-yl]carbonyl}oxy)methyl 2- |
| | [(9R,10S,11S,13S,16R,17R)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-2-oxoethyl rel-butanedioate |
| 69 | ({[1-cyclopropyl-6-fluoro-8-methoxy-7-(octahydro-6H-pyrrolo[3,4- |
| | b]pyridin-6-yl)-4-oxo-1,4-dihydroquinolin-3-yl]carbonyl}oxy)methyl 2- |
| | [(10R,11S,13S,17R)-11,17-dihydroxy-10,13-dimethyl-3-oxo- |
| | 2,3,6,7,8,9,10,11,12,13,14,15,16,17-tetradecahydro-1H- |
| | cyclopenta[a]phenanthren-17-yl]-2-oxoethyl rel-butanedioate |
| 24 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2- |
| | [(9R,10S,11S,13S,16R,17R)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-2-oxoethyl rel-butanedioate |
| 48 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2-[(10R,11S,13S,17R)- |
| | 11,17-dihydroxy-10,13-dimethyl-3-oxo- |
| | 2,3,6,7,8,9,10,11,12,13,14,15,16,17-tetradecahydro-1H- |
| | cyclopenta[a]phenanthren-17-yl]-2-oxoethyl rel-butanedioate |
| 49 | rel-1-cyclopropyl-6-fluoro-7-[1-({[(4-{2-[(9R,10S,11S,13S,16S,17R)- |
| | 9-fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}-4- |
| | oxobutanoyl)oxy]methoxy}carbonyl)octahydro-6H-pyrrolo[3,4- |
| | b]pyridin-6-yl]-8-methoxy-4-oxo-1,4-dihydroquinoline-3-carboxylic |
| | acid |
| 68 | rel-1-cyclopropyl-7-[1-({[(4-{2-[(10R,11S,13S,17R)-11,17-dihydroxy- |
| | 10,13-dimethyl-3-oxo-2,3,6,7,8,9,10,11,12,13,14,15,16,17- |
| | tetradecahydro-1H-cyclopenta[a]phenanthren-17-yl]-2-oxoethoxy}-4- |
| | oxobutanoyl)oxy]methoxy}carbonyl)octahydro-6H-pyrrolo[3,4- |
| | b]pyridin-6-yl]-6-fluoro-8-methoxy-4-oxo-1,4-dihydroquinoline-3- |
| | carboxylic acid |
| 78 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl 2-[(9R,10S,11S,13S,16R,17R)-9-fluoro-11,17- |
| | dihydroxy-10,13,16-trimethyl-3-oxo-6,7,8,10,11,12,13,14,15,16,17- |
| | dodecahydro-3H-cyclopenta[a]phenanthren-17-yl]-2-oxoethyl rel- |
| | butanedioate |
| 76 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl 2-[(9R,10S,11S,13S,16S,17R)-9-fluoro-11,17- |
| | dihydroxy-10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 60 | rel-1-cyclopropyl-7-(1-{[({[4-({2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2- |
| | oxoethoxy}carbonyl)phenyl]carbonyl}oxy)methoxy]carbonyl}octahydro- |
| | 6H-pyrrolo[3,4-b]pyridin-6-yl)-6-fluoro-8-methoxy-4-oxo-1,4- |
| | dihydroquinoline-3-carboxylic acid |
| 27 | rel-1-cyclopropyl-7-(4-{[({[4-({2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2- |
| | oxoethoxy}carbonyl)phenyl]carbonyl}oxy)methoxy]carbonyl}-3- |
| | methylpiperazin-1-yl)-6-fluoro-8-methoxy-4-oxo-1,4-dihydroquinoline- |
| | 3-carboxylic acid |
| 52 | rel-1-cyclopropyl-6-fluoro-7-(1-{[({[4-({2-[(9R,10S,11S,13S,16R,17R)- |
| | 9-fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2- |
| | oxoethoxy}carbonyl)phenyl]carbonyl}oxy)methoxy]carbonyl}octa- |
| | hydro-6H-pyrrolo[3,4-b]pyridin-6-yl)-8-methoxy-4-oxo-1,4- |
| | dihydroquinoline-3-carboxylic acid |
| 12 | rel-1-cyclopropyl-6-fluoro-7-(4-{[({[4-({2-[(9R,10S,11S,13S,16R,17R)- |
| | 9-fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2- |
| | oxoethoxy}carbonyl)phenyl]carbonyl}oxy)methoxy]carbonyl}-3- |
| | methylpiperazin-1-yl)-8-methoxy-4-oxo-1,4-dihydroquinoline-3- |
| | carboxylic acid |
| 50 | ({[1-cyclopropyl-6-fluoro-8-methoxy-7-(octahydro-6H-pyrrolo[3,4- |
| | b]pyridin-6-yl)-4-oxo-1,4-dihydroquinolin-3-yl]carbonyl}oxy)methyl 2- |
| | [(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 8 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2- |
| | [(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 80 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl 2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethyl rel-butanedioate |
| 47 | rel-1-cyclopropyl-7-[4-({[(4-{2-[(10R,11S,13S,17R)-11,17-dihydroxy- |
| | 10,13-dimethyl-3-oxo-2,3,6,7,8,9,10,11,12,13,14,15,16,17- |
| | tetradecahydro-1H-cyclopenta[a]phenanthren-17-yl]-2-oxoethoxy}-4- |
| | oxobutanoyl)oxy]methoxy}carbonyl)-3-methylpiperazin-1-yl]-6-fluoro- |
| | 8-methoxy-4-oxo-1,4-dihydroquinoline-3-carboxylic acid |
| 51 | rel-1-cyclopropyl-6-fluoro-7-[1-({[(4-{2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)oxy]methoxy}carbonyl)octahydro-6H-pyrrolo[3,4- |
| | b]pyridin-6-yl]-8-methoxy-4-oxo-1,4-dihydroquinoline-3-carboxylic |
| | acid |
| 11 | rel-1-cyclopropyl-6-fluoro-7-[4-({[(4-{2-[(9R,10S,11S,13S,16R,17R)- |
| | 9-fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}-4- |
| | oxobutanoyl)oxy]methoxy}carbonyl)-3-methylpiperazin-1-yl]-8- |
| | methoxy-4-oxo-1,4-dihydroquinoline-3-carboxylic acid |
| 26 | rel-1-cyclopropyl-7-[4-({1-[(4-{2-[(10R,11S,13S,17R)-11,17- |
| | dihydroxy-10,13-dimethyl-3-oxo-6,7,8,9,10,11, 12,13,14,15,16,17- |
| | dodecahydro-3H-cyclopenta[a]phenanthren-17-yl]-2-oxoethoxy}-4- |
| | oxobutanoyl)oxy]ethoxy}carbonyl)-3-methylpiperazin-1-yl]-6-fluoro-8- |
| | methoxy-4-oxo-1,4-dihydroquinoline-3-carboxylic acid |
| 66 | ({[1-cyclopropyl-6-fluoro-8-methoxy-7-(octahydro-6H-pyrrolo[3,4- |
| | b]pyridin-6-yl)-4-oxo-1,4-dihydroquinolin-3-yl]carbonyl}oxy)methyl 2- |
| | [(8R,10S,11R,13R,14R,17S)-11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 59 | rel-1-cyclopropyl-7-[1-({[(4-{2-[(8R,9R,10S,11R,13R,14R,17S)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)oxy]methoxy}carbonyl)octahydro-6H-pyrrolo[3,4- |
| | b]pyridin-6-yl]-6-fluoro-8-methoxy-4-oxo-1,4-dihydroquinoline-3- |
| | carboxylic acid |
| 44 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2- |
| | [(8R,10S,11R,13R,14R,17S)-11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 25 | rel-1-cyclopropyl-7-[4-({[(4-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-4- |
| | oxobutanoyl)oxy]methoxy}carbonyl)-3-methylpiperazin-1-yl]-6-fluoro- |
| | 8-methoxy-4-oxo-1,4-dihydroquinoline-3-carboxylic acid |
| 43 | 1-[({2-[(8R,10S,11R,13R,14R,17S)-11,17-dihydroxy-10,13-dimethyl- |
| | 3-oxo-6,7,8,9,10,11,12,13,14,15,16,17-dodecahydro-3H- |
| | cyclopenta[a]phenanthren-17-yl]-2-oxoethoxy}carbonyl)oxy]ethyl rel- |
| | 1-cyclopropyl-6-fluoro-8-methoxy-7-(3-methylpiperazin-1-yl)-4-oxo- |
| | 1,4-dihydroquinoline-3-carboxylate |
| 42 | [({2-[(8R,10S,11R,13R,14R,17S)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethoxy}carbonyl)oxy]methyl |
| | rel-1-cyclopropyl-6-fluoro-8-methoxy-7-(3-methylpiperazin-1-yl)-4- |
| | oxo-1,4-dihydroquinoline-3-carboxylate |
| 65 | 1-[({2-[(8R,10S,11R,13R,14R,17S)-11,17-dihydroxy-10,13-dimethyl- |
| | 3-oxo-6,7,8,9,10,11,12,13,14,15,16,17-dodecahydro-3H- |
| | cyclopenta[a]phenanthren-17-yl]-2-oxoethoxy}carbonyl)oxy]ethyl rel- |
| | 1-cyclopropyl-6-fluoro-8-methoxy-7-[(4aS,7aS)-octahydro-6H- |
| | pyrrolo[3,4-b]pyridin-6-yl]-4-oxo-1,4-dihydroquinoline-3-carboxylate |
| 64 | [({2-[(8R,10S,11R,13R,14R,17S)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethoxy}carbonyl)oxy]methyl |
| | rel-1-cyclopropyl-6-fluoro-8-methoxy-7-[(4aS,7aS)-octahydro-6H- |
| | pyrrolo[3,4-b]pyridin-6-yl]-4-oxo-1,4-dihydroquinoline-3-carboxylate |
| 75 | (2R,3R)-2-[(dichloroacetyl)amino]-3-hydroxy-3-(4-nitrophenyl)propyl |
| | 2-[(8S,10R,11S,13S,14S,17R)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethyl rel-butanedioate |
| 2 | rel-1-cyclopropyl-6-fluoro-7-[4-({[(4-{2-[(9R,10S,11S,13S,16S,17R)- |
| | 9-fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}-4- |
| | oxobutanoyl)oxy]methoxy}carbonyl)-3-methylpiperazin-1-yl]-8- |
| | methoxy-4-oxo-1,4-dihydroquinoline-3-carboxylic acid |
| 79 | 4-{[(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methoxy}-4-oxobutyl 2-[(10R,11S,13S,17R)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 83 | 4-{[(2R,3S,4R,5R,6S)-4-amino-6-{[(1S,2S,3R,4S,6R)-4-amino-6- |
| | {[(2S)-4-amino-2-hydroxybutanoyl]amino}-3-{[(2R,3R,4S,5S,6R)-6- |
| | (aminomethyl)-3,4,5-trihydroxytetrahydro-2H-pyran-2-yl]oxy}-2- |
| | hydroxycyclohexyl]oxy}-5-hydroxy-2-(hydroxymethyl)tetrahydro-2H- |
| | pyran-3-yl]oxy}-4-oxobutyl 2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethyl rel-butanedioate |
| 82 | 4-{[(2R,3S,4S,5R,6S)-4-amino-6-{[(1S,2S,3R,4S,6R)-4-amino-6- |
| | {[(2S)-4-amino-2-hydroxybutanoyl]amino}-3-{[(2R,3R,4S,5S,6R)-6- |
| | (aminomethyl)-3,4,5-trihydroxytetrahydro-2H-pyran-2-yl]oxy}-2- |
| | hydroxycyclohexyl]oxy}-3,5-dihydroxytetrahydro-2H-pyran-2- |
| | yl]methoxy}-4-oxobutyl 2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethyl rel-butanedioate |
| 7 | 2-{2-[(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}ethyl |
| | rel-1-cyclopropyl-6-fluoro-8-methoxy-7-(3-methylpiperazin-1-yl)-4- |
| | oxo-1,4-dihydroquinoline-3-carboxylate |
| 77 | 4-{[(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methoxy}-4-oxobutyl 2-[(9R,10S,11S,13S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 6 | 4-{2-[(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutyl rel-1-cyclopropyl-6-fluoro-8-methoxy-7-(3-methylpiperazin- |
| | 1-yl)-4-oxo-1,4-dihydroquinoline-3-carboxylate |
| 57 | 4-{2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutyl rel-1-cyclopropyl-6-fluoro-8-methoxy-7-[(4aS,7aS)- |
| | octahydro-6H-pyrrolo[3,4-b]pyridin-6-yl]-4-oxo-1,4-dihydroquinoline- |
| | 3-carboxylate |
| 23 | 4-{2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutyl rel-1-cyclopropyl-6-fluoro-8-methoxy-7-(3-methylpiperazin- |
| | 1-yl)-4-oxo-1,4-dihydroquinoline-3-carboxylate |
| 63 | 4-{2-[(8R,10S,11R,13R,14R,17S)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethoxy}-4-oxobutyl rel-1- |
| | cyclopropyl-6-fluoro-8-methoxy-7-[(4aS,7aS)-octahydro-6H- |
| | pyrrolo[3,4-b]pyridin-6-yl]-4-oxo-1,4-dihydroquinoline-3-carboxylate |
| 41 | 4-{2-[(8R,10S,11R,13R,14R,17S)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethoxy}-4-oxobutyl rel-1- |
| | cyclopropyl-6-fluoro-8-methoxy-7-(3-methylpiperazin-1-yl)-4-oxo-1,4- |
| | dihydroquinoline-3-carboxylate |
| 62 | 2-[(8R,9R,10S,11R,13R,14R,17S)-11,17-dihydroxy-10,13-dimethyl- |
| | 3-oxo-6,7,8,9,10,11,12,13,14,15,16,17-dodecahydro-3H- |
| | cyclopenta[a]phenanthren-17-yl]-2-oxoethyl rel-4-[({1-cyclopropyl-7- |
| | [(4aS,7aS)-4a,7a-dimethyloctahydro-6H-pyrrolo[3,4-b]pyridin-6-yl]-6- |
| | fluoro-8-methoxy-4-oxo-1,4-dihydroquinolin-3- |
| | yl}carbonyl)amino]butanoate |
| 5 | 2-{2-[2-({[1-cyclopropyl-6-fluoro-8-methoxy-7-(3-methylpiperazin-1- |
| | yl)-4-oxo-1,4-dihydroquinolin-3-yl]carbonyl}oxy)ethoxy]ethoxy}ethyl |
| | 2-[(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 56 | 3-[({1-cyclopropyl-6-fluoro-8-methoxy-7-[(4aR,7aR)-octahydro-6H- |
| | pyrrolo[3,4-b]pyridin-6-yl]-4-oxo-1,4-dihydroquinolin-3- |
| | yl}carbonyl)oxy]propyl 2-[(8S,9R,10S,11S,13S,14S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 55 | 3-[({1-cyclopropyl-6-fluoro-8-methoxy-7-[(4aR,7aR)-octahydro-6H- |
| | pyrrolo[3,4-b]pyridin-6-yl]-4-oxo-1,4-dihydroquinolin-3- |
| | yl}carbonyl)oxy]propyl 2-[(8S,9R,10S,11S,13S,14S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethyl rel-(2E)-but-2-enedioate |
| 54 | 3-[({1-cyclopropyl-6-fluoro-8-methoxy-7-[(4aR,7aR)-octahydro-6H- |
| | pyrrolo[3,4-b]pyridin-6-yl]-4-oxo-1,4-dihydroquinolin-3- |
| | yl}carbonyl)oxy]propyl 2-[(8S,9R,10S,11S,13S,14S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethyl rel-benzene-1,4- |
| | dicarboxylate |
| 22 | 4-({2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2- |
| | oxoethoxy}carbonyl)benzyl rel-1-cyclopropyl-6-fluoro-8-methoxy-7- |
| | (3-methylpiperazin-1-yl)-4-oxo-1,4-dihydroquinoline-3-carboxylate |
| 74 | (2R,3R)-2-[(dichloroacetyl)amino]-3-hydroxy-3-(4-nitrophenyl)propyl |
| | 2-[(8S,9R,10S,11S,13S,14S,16R,17R)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | benzene-1,4-dicarboxylate |
| 21 | 2-({[1-cyclopropyl-6-fluoro-8-methoxy-7-(3-methylpiperazin-1-yl)-4- |
| | oxo-1,4-dihydroquinolin-3-yl]carbonyl}oxy)ethyl 2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | (2E)-but-2-enedioate |
| 20 | 2-({[1-cyclopropyl-6-fluoro-8-methoxy-7-(3-methylpiperazin-1-yl)-4- |
| | oxo-1,4-dihydroquinolin-3-yl]carbonyl}oxy)ethyl 2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | benzene-1,4-dicarboxylate |
| 4 | 4-({2-[(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2- |
| | oxoethoxy}carbonyl)benzyl rel-1-cyclopropyl-6-fluoro-8-methoxy-7- |
| | (3-methylpiperazin-1-yl)-4-oxo-1,4-dihydroquinoline-3-carboxylate |
| 19 | 2-{2-[2-({[1-cyclopropyl-6-fluoro-8-methoxy-7-(3-methylpiperazin-1- |
| | yl)-4-oxo-1,4-dihydroquinolin-3-yl]carbonyl}oxy)ethoxy]ethoxy}ethyl |
| | 2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 40 | 2-[(8R,9R,10S,11R,13R,14R,17S)-11,17-dihydroxy-10,13-dimethyl- |
| | 3-oxo-6,7,8,9,10,11,12,13,14,15,16,17-dodecahydro-3H- |
| | cyclopenta[a]phenanthren-17-yl]-2-oxoethyl rel-4-({[1-cyclopropyl-6- |
| | fluoro-8-methoxy-7-(3-methylpiperazin-1-yl)-4-oxo-1,4- |
| | dihydroquinolin-3-yl]carbonyl}amino)butanoate |
| 3 | 3-({[1-cyclopropyl-6-fluoro-8-methoxy-7-(3-methylpiperazin-1-yl)-4- |
| | oxo-1,4-dihydroquinolin-3-yl]carbonyl}oxy)propyl 2- |
| | [(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 73 | (2R,3R)-2-[(dichloroacetyl)amino]-3-hydroxy-3-(4-nitrophenyl)propyl |
| | 2-[(8S,9R,10S,11S,13S,14S,16S,17R)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 18 | 3-({[1-cyclopropyl-6-fluoro-8-methoxy-7-(3-methylpiperazin-1-yl)-4- |
| | oxo-1,4-dihydroquinolin-3-yl]carbonyl}oxy)propyl 2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 17 | 2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel-3- |
| | ({[1-cyclopropyl-6-fluoro-8-methoxy-7-(3-methylpiperazin-1-yl)-4-oxo- |
| | 1,4-dihydroquinolin-3-yl]carbonyl}amino)propanoate |
| 10 | rel-1-cyclopropyl-6-fluoro-7-[4-(4-{2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)-3-methylpiperazin-1-yl]-8-methoxy-4-oxo-1,4- |
| | dihydroquinoline-3-carboxylic acid |
| 9 | rel-1-cyclopropyl-6-fluoro-7-[4-(2-{2- |
| | [(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-2- |
| | oxoethyl)-3-methylpiperazin-1-yl]-8-methoxy-4-oxo-1,4- |
| | dihydroquinoline-3-carboxylic acid |
| 16 | 2-{2-[(8R,9S,10R,11R,13R,14R,16S,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-2- |
| | oxoethyl rel-1-cyclopropyl-6-fluoro-8-methoxy-7-(3-methylpiperazin- |
| | 1-yl)-4-oxo-1,4-dihydroquinoline-3-carboxylate |
| 1 | rel-1-cyclopropyl-6-fluoro-7-[4-(4-{2- |
| | [(8R,9S,10R,11R,13R,14R,16R,17S)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)-3-methylpiperazin-1-yl]-8-methoxy-4-oxo-1,4- |
| | dihydroquinoline-3-carboxylic acid |
| 71 | [({7-[(3R)-3-aminoazepan-1-yl]-8-chloro-1-cyclopropyl-6-fluoro-4-oxo- |
| | 1,4-dihydroquinolin-3-yl}carbonyl)oxy]methyl 2- |
| | [(8S,10R,11S,13S,14S,17R)-11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 84 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl 2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethyl rel-4,18-dioxo- |
| | 5,8,11,14,17-pentaoxahenicosane-1,21-dioate |
| 85 | [(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl 2-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-2-oxoethyl rel-4,15-dioxo-5,8,11,14- |
| | tetraoxaoctadecane-1,18-dioate |
| 86 | 1-{[(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methyl}4′-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethyl} 1′,4-methanediyl |
| | reldi-butanedioate |
| 87 | rel-1-cyclopropyl-6-fluoro-7-(1-{12-[(9R,10S,11S,13S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-5,9,12-trioxo-2,4,7,10- |
| | tetraoxadodecan-1-oyl}octahydro-6H-pyrrolo[3,4-b]pyridin-6-yl)-8- |
| | methoxy-4-oxo-1,4-dihydroquinoline-3-carboxylic acid |
| 88 | rel-1-cyclopropyl-6-fluoro-7-(4-{12-[(9R,10S,11S,13S,16R,17R)-9- |
| | fluoro-11,17-dihydroxy-10,13,16-trimethyl-3-oxo- |
| | 6,7,8,9,10,11,12,13,14,15,16,17-dodecahydro-3H- |
| | cyclopenta[a]phenanthren-17-yl]-5,9,12-trioxo-2,4,7,10- |
| | tetraoxadodecan-1-oyl}-3-methylpiperazin-1-yl)-8-methoxy-4-oxo- |
| | 1,4-dihydroquinoline-3-carboxylic acid |
| 89 | rel-7-(1-{7-carboxy-15-[(10R,11S,13S,17R)-11,17-dihydroxy-10,13- |
| | dimethyl-3-oxo-6,7,8,9,10,11,12,13,14,15,16,17-dodecahydro-3H- |
| | cyclopenta[a]phenanthren-17-yl]-5,9,12,15-tetraoxo-2,4,13-trioxa-8- |
| | azapentadecan-1-oyl}octahydro-6H-pyrrolo[3,4-b]pyridin-6-yl)-1- |
| | cyclopropyl-6-fluoro-8-methoxy-4-oxo-1,4-dihydroquinoline-3- |
| | carboxylic acid |
| 90 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2- |
| | [(9R,10S,11S,13S,16R,17R)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-2-oxoethyl rel-benzene-1,4- |
| | dicarboxylate |
| 91 | rel-1-cyclopropyl-7-(1-{12-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-5,9,12-trioxo-2,4,7,10- |
| | tetraoxadodecan-1-oyl}octahydro-6H-pyrrolo[3,4-b]pyridin-6-yl)-6- |
| | fluoro-8-methoxy-4-oxo-1,4-dihydroquinoline-3-carboxylic acid |
| 92 | rel-1-cyclopropyl-7-(4-{12-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-5,9,12-trioxo-2,4,7,10- |
| | tetraoxadodecan-1-oyl}-3-methylpiperazin-1-yl)-6-fluoro-8-methoxy- |
| | 4-oxo-1,4-dihydroquinoline-3-carboxylic acid |
| 93 | 1-{2-[(10R,11S,13S,17R)-11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl} 4-{[({2- |
| | [(9R,10S,11S,13S,16S,17R)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-2-oxoethoxy}carbonyl)oxy]methyl} |
| | rel-(2R)-2-aminobutanedioate |
| 94 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-3- |
| | hydroxypentanedioate |
| 95 | rel-1-cyclopropyl-7-[1-({[(5-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-3-hydroxy-5- |
| | oxopentanoyl)oxy]methoxy}carbonyl)octahydro-6H-pyrrolo[3,4- |
| | b]pyridin-6-yl]-6-fluoro-8-methoxy-4-oxo-1,4-dihydroquinoline-3- |
| | carboxylic acid |
| 96 | rel-1-cyclopropyl-7-[4-({[(5-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-3-hydroxy-5- |
| | oxopentanoyl)oxy]methoxy}carbonyl)-3-methylpiperazin-1-yl]-6- |
| | fluoro-8-methoxy-4-oxo-1,4-dihydroquinoline-3-carboxylic acid |
| 97 | rel-7-{4-[(2R)-2-amino-5-{2-[(10S,11R,13R,17S)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-5-oxopentanoyl]- |
| | 3-methylpiperazin-1-yl}-1-cyclopropyl-6-fluoro-8-methoxy-4-oxo-1,4- |
| | dihydroquinoline-3-carboxylic acid |
| 98 | ({[1-cyclopropyl-6-fluoro-8-methoxy-7-(3-methylpiperazin-1-yl)-4-oxo- |
| | 1,4-dihydroquinolin-3-yl]carbonyl}oxy)methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-3- |
| | aminopentanedioate |
| 99 | rel-1-cyclopropyl-7-{4-[(7R)-7-(2-{2-[(10S,11R,13R,17S)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethoxy}-2- |
| | oxoethyl)-11,11-dimethyl-5,9-dioxo-2,4,10-trioxa-8-azadodecan-1- |
| | oyl]-3-methylpiperazin-1-yl}-6-fluoro-8-methoxy-4-oxo-1,4- |
| | dihydroquinoline-3-carboxylic acid |
| 100 | rel-7-(4-{2-[(tert-butoxycarbonyl)amino]-3-[(4-{2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethoxy}-4- |
| | oxobutanoyl)oxy]propanoyl}-3-methylpiperazin-1-yl)-1-cyclopropyl-6- |
| | fluoro-8-methoxy-4-oxo-1,4-dihydroquinoline-3-carboxylic acid |
| 101 | rel-1-cyclopropyl-7-{4-[7-({2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}carbonyl)-11,11- |
| | dimethyl-5,9-dioxo-2,4,10-trioxa-8-azadodecan-1-oyl]-3- |
| | methylpiperazin-1-yl}-6-fluoro-8-methoxy-4-oxo-1,4-dihydroquinoline- |
| | 3-carboxylic acid |
| 102 | rel-1-cyclopropyl-7-[4-(6-{[(4-{2-[(10R,11S,13S,17R)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)oxy]methyl}-10,10-dimethyl-5,8-dioxo-2,4,9-trioxa-7- |
| | azaundecan-1-oyl)-3-methylpiperazin-1-yl]-6-fluoro-8-methoxy-4- |
| | oxo-1,4-dihydroquinoline-3-carboxylic acid |
| 103 | rel-1-cyclopropyl-6-fluoro-7-{1-[(10R)-14- |
| | [(9S,10R,11R,13R,16S,17S)-9-fluoro-11,17-dihydroxy-10,13,16- |
| | trimethyl-3-oxo-6,7,8,9,10,11,12,13,14,15,16,17-dodecahydro-3H- |
| | cyclopenta[a]phenanthren-17-yl]-5,8,11,14-tetraoxo-10-(propan-2-yl)- |
| | 2,4,12-trioxa-9-azatetradecan-1-oyl]octahydro-6H-pyrrolo[3,4- |
| | b]pyridin-6-yl}-8-methoxy-4-oxo-1,4-dihydroquinoline-3-carboxylic |
| | acid |
| 104 | rel-1-cyclopropyl-7-{4-[6-(2-{2-[(10R,11S,13S,17R)-11,17-dihydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-2-oxoethyl)-10,10- |
| | dimethyl-5,8-dioxo-2,4,9-trioxa-7-azaundecan-1-oyl]-3- |
| | methylpiperazin-1-yl}-6-fluoro-8-methoxy-4-oxo-1,4-dihydroquinoline- |
| | 3-carboxylic acid |
| 105 | [({[(2R,3S,4S,5R,6S)-4-amino-6-{[(1S,2S,3R,4S,6R)-4,6-diamino-3- |
| | {[(2R,3R,5S,6R)-3-amino-6-(aminomethyl)-5-hydroxytetrahydro-2H- |
| | pyran-2-yl]oxy}-2-hydroxycyclohexyl]oxy}-3,5-dihydroxytetrahydro- |
| | 2H-pyran-2-yl]methoxy}carbonyl)oxy]methyl 2-[(10R,11S,13S,17R)- |
| | 11,17-dihydroxy-10,13-dimethyl-3-oxo- |
| | 6,7,8,9,10,11,12,13,14,15,16,17-dodecahydro-3H- |
| | cyclopenta[a]phenanthren-17-yl]-2-oxoethyl rel-butanedioate |
| 106 | {[(1-cyclopropyl-6-fluoro-8-methoxy-7-{1-[(5-methyl-2-oxo-1,3-dioxol- |
| | 4-yl)methyl]octahydro-6H-pyrrolo[3,4-b]pyridin-6-yl}-4-oxo-1,4- |
| | dihydroquinolin-3-yl)carbonyl]oxy}methyl 2- |
| | [(8R,10S,11R,13R,14R,17S)-11,17-dihydroxy-10,13-dimethyl-3-oxo- |
| | 2,3,6,7,8,9,10,11,12,13,14,15,16,17-tetradecahydro-1H- |
| | cyclopenta[a]phenanthren-17-yl]-2-oxoethyl rel-butanedioate |
| 107 | {[(1-cyclopropyl-6-fluoro-8-methoxy-7-{3-methyl-4-[(5-methyl-2-oxo- |
| | 1,3-dioxol-4-yl)methyl]piperazin-1-yl}-4-oxo-1,4-dihydroquinolin-3- |
| | yl)carbonyl]oxy}methyl 2-[(8R,10S,11R,13R,14R,17S)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 108 | 2-[2-(2-{[(1-cyclopropyl-6-fluoro-8-methoxy-7-{3-methyl-4-[(5-methyl- |
| | 2-oxo-1,3-dioxol-4-yl)methyl]piperazin-1-yl}-4-oxo-1,4- |
| | dihydroquinolin-3-yl)carbonyl]oxy}ethoxy)ethoxy]ethyl 2- |
| | [(8R,9R,10S,11R,13R,14R,17S)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl]-2-oxoethyl rel-butanedioate |
| 109 | 1-cyclopropyl-7-(4-{[({[1-cyclopropyl-6-fluoro-8-methoxy-7- |
| | (octahydro-6H-pyrrolo[3,4-b]pyridin-6-yl)-4-oxo-1,4-dihydroquinolin- |
| | 3-yl]carbonyl}oxy)methoxy]carbonyl}-3-methylpiperazin-1-yl)-6- |
| | fluoro-8-methoxy-4-oxo-1,4-dihydroquinoline-3-carboxylic acid |
| 110 | 8-chloro-1-cyclopropyl-7-{3-[({[(4-{2-[(10R,11S,13S,17R)-11,17- |
| | dihydroxy-10,13-dimethyl-3-oxo-6,7,8,9,10,11,12,13,14,15,16,17- |
| | dodecahydro-3H-cyclopenta[a]phenanthren-17-yl]-2-oxoethoxy}-4- |
| | oxobutanoyl)oxy]methoxy}carbonyl)amino]azepan-1-yl}-6-fluoro-4- |
| | oxo-1,4-dihydroquinoline-3-carboxylic acid |
| 111 | 4-{2-[(8R,9S,10R,11R,13R,14R,16R,17S)-17-(butanoyloxy)-9-fluoro- |
| | 11-hydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}-4-oxobutyl rel-1- |
| | cyclopropyl-6-fluoro-8-methoxy-7-(3-methylpiperazin-1-yl)-4-oxo-1,4- |
| | dihydroquinoline-3-carboxylate |
| 112 | 4-{[(8R,9S,10R,11R,13R,14R,16R,17S)-17-{[(2,2- |
| | dimethylpropanoyl)oxy]acetyl}-9-fluoro-11-hydroxy-10,13,16- |
| | trimethyl-3-oxo-6,7,8,9,10,11,12,13,14,15,16,17-dodecahydro-3H- |
| | cyclopenta[a]phenanthren-17-yl]oxy}-4-oxobutyl rel-1-cyclopropyl-6- |
| | fluoro-8-methoxy-7-(3-methylpiperazin-1-yl)-4-oxo-1,4- |
| | dihydroquinoline-3-carboxylate |
| 113 | 4-{[(8R,9S,10R,11R,13R,14R,16R,17S)-9-fluoro-11-hydroxy- |
| | 10,13,16-trimethyl-17-{[(2-methylpropanoyl)oxy]acetyl}-3-oxo- |
| | 6,7,8,9,10,11,12,13,14,15,16,17-dodecahydro-3H- |
| | cyclopenta[a]phenanthren-17-yl]oxy}-4-oxobutyl rel-1-cyclopropyl-6- |
| | fluoro-8-methoxy-7-(3-methylpiperazin-1-yl)-4-oxo-1,4- |
| | dihydroquinoline-3-carboxylate |
| 114 | 4-{2-[(8R,9S,10R,11R,13R,14R,16S,17S)-17-(butanoyloxy)-9-fluoro- |
| | 11-hydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}-4-oxobutyl rel-1- |
| | cyclopropyl-6-fluoro-8-methoxy-7-[(4aS,7aS)-octahydro-6H- |
| | pyrrolo[3,4-b]pyridin-6-yl]-4-oxo-1,4-dihydroquinoline-3-carboxylate |
| 115 | 4-{[(8R,10S,11R,13R,14R,17S)-17-{[(2,2- |
| | dimethylpropanoyl)oxy]acetyl}-11-hydroxy-10,13-dimethyl-3-oxo- |
| | 6,7,8,9,10,11,12,13,14,15,16,17-dodecahydro-3H- |
| | cyclopenta[a]phenanthren-17-yl]oxy}-4-oxobutyl rel-1-cyclopropyl-6- |
| | fluoro-8-methoxy-7-(3-methylpiperazin-1-yl)-4-oxo-1,4- |
| | dihydroquinoline-3-carboxylate |
| 116 | 4-({(8R,10S,11R,13R,14R,17S)-17-[(acetyloxy)acetyl]-11-hydroxy- |
| | 10,13-dimethyl-3-oxo-6,7,8,9,10,11,12,13,14,15,16,17-dodecahydro- |
| | 3H-cyclopenta[a]phenanthren-17-yl}oxy)-4-oxobutyl rel-1- |
| | cyclopropyl-6-fluoro-8-methoxy-7-(3-methylpiperazin-1-yl)-4-oxo-1,4- |
| | dihydroquinoline-3-carboxylate |
| 117 | 4-({(8R,9S,10R,11R,13R,14R,16S,17S)-17-[(acetyloxy)acetyl]-9- |
| | fluoro-11-hydroxy-10,13,16-trimethyl-3-oxo- |
| | 6,7,8,9,10,11,12,13,14,15,16,17-dodecahydro-3H- |
| | cyclopenta[a]phenanthren-17-yl}oxy)-4-oxobutyl rel-1-cyclopropyl-6- |
| | fluoro-8-methoxy-7-(3-methylpiperazin-1-yl)-4-oxo-1,4- |
| | dihydroquinoline-3-carboxylate |
| 118 | 4-(2-{(8R,10S,11R,13R,14R,17S)-11-hydroxy-10,13-dimethyl-3-oxo- |
| | 17-[(phenylcarbonyl)oxyl-6,7,8,9,10,11,12,13,14,15,16,17- |
| | dodecahydro-3H-cyclopenta[a]phenanthren-17-yl}-2-oxoethoxy)-4- |
| | oxobutyl rel-1-cyclopropyl-6-fluoro-8-methoxy-7-(3-methylpiperazin- |
| | 1-yl)-4-oxo-1,4-dihydroquinoline-3-carboxylate |
| 119 | 4-{2-[(8R,10S,11R,13R,14R,17S)-17-(butanoyloxy)-11-hydroxy- |
| | 10,13-dimethyl-3-oxo-6,7,8,9,10,11,12,13,14,15,16,17-dodecahydro- |
| | 3H-cyclopenta[a]phenanthren-17-yl]-2-oxoethoxy}-4-oxobutyl rel-1- |
| | cyclopropyl-6-fluoro-8-methoxy-7-(3-methylpiperazin-1-yl)-4-oxo-1,4- |
| | dihydroquinoline-3-carboxylate |
| 120 | 4-({2-[(8R,9S,10R,11R,13R,14R,16R,17S)-17-(acetyloxy)-9-fluoro- |
| | 11-hydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}carbonyl)benzyl rel-1- |
| | cyclopropyl-6-fluoro-8-methoxy-7-(3-methylpiperazin-1-yl)-4-oxo-1,4- |
| | dihydroquinoline-3-carboxylate |
| 121 | 4-({2-[(8R,9S,10R,11R,13R,14R,16R,17S)-17-(acetyloxy)-9-fluoro- |
| | 11-hydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}carbonyl)benzyl rel-1- |
| | cyclopropyl-6-fluoro-8-methoxy-7-(3-methylpiperazin-1-yl)-4-oxo-1,4- |
| | dihydroquinoline-3-carboxylate |
| 122 | 4-({2-[(8R,9S,10R,11R,13R,14R,16R,17S)-17-(acetyloxy)-9-fluoro- |
| | 11-hydroxy-10,13,16-trimethyl-3-oxo- |
| | 6,7,8,9,10,11,12,13,14,15,16,17-dodecahydro-3H- |
| | cyclopenta[a]phenanthren-17-yl]-2-oxoethoxy}carbonyl)benzyl rel-1- |
| | cyclopropyl-6-fluoro-8-methoxy-7-(3-methylpiperazin-1-yl)-4-oxo-1,4- |
| | dihydroquinoline-3-carboxylate |
| 123 | 4-{[(8R,10S,11R,13R,14R,17S)-11-hydroxy-10,13-dimethyl-17-{[(2- |
| | methylpropanoyl)oxy]acetyl}-3-oxo-6,7,8,9,10,11,12,13,14,15,16,17- |
| | dodecahydro-3H-cyclopenta[a]phenanthren-17-yl]oxy}-4-oxobutyl rel- |
| | 1-cyclopropyl-6-fluoro-8-methoxy-7-(3-methylpiperazin-1-yl)-4-oxo- |
| | 1,4-dihydroquinoline-3-carboxylate |
| 124 | (9R,10S,11S,13S,16S,17R)-17-[(acetyloxy)acetyl]-9-fluoro-17- |
| | hydroxy-10,13,16-trimethyl-3-oxo-6,7,8,9,10,11,12,13,14,15,16,17- |
| | dodecahydro-3H-cyclopenta[a]phenanthren-11-yl rel-3-({[1- |
| | cyclopropyl-6-fluoro-8-methoxy-7-(3-methylpiperazin-1-yl)-4-oxo-1,4- |
| | dihydroquinolin-3-yl]carbonyl}amino)propanoate |
| 125 | 4-({(8R,9S,10R,11R,13R,14R,16R,17S)-17-[(acetyloxy)acetyl]-9- |
| | fluoro-11-hydroxy-10,13,16-trimethyl-3-oxo- |
| | 6,7,8,9,10,11,12,13,14,15,16,17-dodecahydro-3H- |
| | cyclopenta[a]phenanthren-17-yl}oxy)-4-oxobutyl rel-1-cyclopropyl-6- |
| | fluoro-8-methoxy-7-(3-methylpiperazin-1-yl)-4-oxo-1,4- |
| | dihydroquinoline-3-carboxylate |
| 126 | (2R,3R)-2-[(dichloroacetyl)amino]-3-hydroxy-3-(4-nitrophenyl)propyl |
| | 2-[(8S,9R,10S,11S,13S,14S,16S,17R)-9-fluoro-11,17-dihydroxy- |
| | 10,13,16-trimethyl-3-oxo-6,7,8,9,10,11,12,13,14,15,16,17- |
| | dodecahydro-3H-cyclopenta[a]phenanthren-17-yl]-2-oxoethyl rel- |
| | butanedioate |
| 127 | (2R,3R,4S,5R,6R)-6-((1S)-2-chloro-1-(1-methyl-4-propylpyrrolidine- |
| | 2-carboxamido)propyl)-4,5-dihydroxy-2-(methylthio)tetrahydro-2H- |
| | pyran-3-yl (2-((10R,11S,13S,17R)-11,17-dihydroxy-10,13-dimethyl-3- |
| | oxo-6,7,8,9,10,11,12,13,14,15,16,17-dodecahydro-3H- |
| | cyclopenta[a]phenanthren-17-yl)-2-oxoethyl) succinate |

Some compounds of the invention have at least one stereogenic center in their structure. This stereogenic center may be present in an R or S configuration, said R and S notation is used in correspondence with the rules described in Pure Appli. Chem. (1976), 45, 11-13.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of the invention are able to form.

The acid addition salt form of a compound of the invention that occurs in its free form as a base can be obtained by treating the free base with an appropriate acid such as an inorganic acid, for example, hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic acid and the like (Handbook of Pharmaceutical Salts, P. Heinrich Stahl & Camille G. Wermuth (Eds), Verlag Helvetica Chimica Acta—Zürich, 2002, 329-345).

The base addition salt form of a compound of the invention that occurs in its acid form can be obtained by treating the acid with an appropriate base such as an inorganic base, for example, sodium hydroxide, magnesium hydroxide, potassium hydroxide, Calcium hydroxide, ammonia and the like; or an organic base such as for example, L-Arginine, ethanolamine, betaine, benzathine, morpholine and the like. (Handbook of Pharmaceutical Salts, P. Heinrich Stahl & Camille G. Wermuth (Eds), Verlag Helvetica Chimica Acta—Zürich, 2002, 329-345).

Compounds of the invention and their salts can be in the form of a solvate, which is included within the scope of the present invention. Such solvates include for example hydrates, alcoholates and the like.

The compounds of the invention are indicated for use in treating or preventing conditions conjunctivitis, keratitis, blepharitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis.

These compounds are useful for the treatment of mammals, including humans, with a range of conditions and diseases which are alleviated by an antibiotic and steroid drug.

In still another embodiment of the invention, there are provided methods for treating or preventing eye conditions such as: conjunctivitis, keratitis, endophthalmitis, blepharitis, dacyrocystitis, hordeolum, corneal ulcers, anterior blepharitis, red eye, hyperemia, posterior blepharitis, meibomian gland dysfunction, dry eye disease (keratocojunctivitis sicca) ocular pain, ocular pain and inflammation post-ocular surgery, bacterial conjunctivitis, anterior uveitis, in a patient suffering thereof. Such methods can be performed, for example, by administering to a subject in need thereof a therapeutically effective amount of at least one compound of the invention, or any combination thereof, or pharmaceutically acceptable salts, hydrates, solvates, crystal forms thereof.

The present invention concerns the use of a compound of the invention or a pharmaceutically acceptable salt thereof, for the manufacture of a medicament for the treatment of conjunctivitis, keratitis, blepharitis, dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

The compounds of the invention may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

The compounds of the invention may also be administered as pharmaceutical compositions in a form suitable for topical use, for example, as oily suspensions, as solutions or suspensions in aqueous liquids or nonaqueous liquids, or as oil-in-water or water-in-oil liquid emulsions.

Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants.

Preferred preservatives that may be used in the pharmaceutical compositions of the present invention include, but are not limited to, benzalkonium chloride, chlorobutanol, thimerosal, phenylmercuric acetate and phenylmercuric nitrate.

A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 0-10 |
| buffer | 0.01-10 |
| pH adjustor | q.s. pH 4.5-7.8 |
| antioxidant | as needed |
| surfactant | as needed |
| purified water | to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses. Especially preservative-free solutions are often formulated in non-resealable containers containing up to about ten, preferably up to about five units doses, where a typical unit dose is from one to about 8 drops, preferably one to about 3 drops. The volume of one drop usually is about 20-35 μl.

Since individual subjects may present a wide variation in severity of symptoms and each drug has its unique therapeutic characteristics, the precise mode of administration and dosage employed for each subject is left to the discretion of the practitioner.

The patient may be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The compounds and pharmaceutical compositions described herein are useful as medicaments in mammals, including humans, for treatment of diseases and/or alleviations of conditions such as conjunctivitis, keratitis, blepharitis, endophthalmitis, dacyrocystitis, hordeolum, corneal ulcers, anterior blepharitis, red eye, posterior blepharitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, dry eye, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

Thus, in further embodiments of the invention, there are provided methods for treating conjunctivitis, keratitis, blepharitis, endophthalmitis dacyrocystitis, hordeolum, corneal ulcers, anterior blepharitis, posterior blepharitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, dry eye, blepharitis, endophthalmitis, meibomian gland dysfunction, superficial punctate keratitis, herpes zoster keratitis, iritis, cyclitis, selected infective conjunctivitis, corneal injury from chemical radiation, or thermal burns, penetration of foreign bodies, allergy, and combinations thereof.

Such methods can be performed, for example, by administering to a subject in need thereof a pharmaceutical composition containing a therapeutically effective amount of at least one invention compound. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

The present invention concerns also processes for preparing the compounds of the invention. The compounds according to the invention can be prepared analogously to conventional methods as understood by the person skilled in the art of synthetic organic chemistry. The Schemes set forth below, illustrate how the compounds according to the invention can be made. It should be noted that the brief description on each of the arrows for each conversion has been added for illustration purpose sonly and should not be regarded as limiting with respect to the sequence of each individual step.

It is to be understood that both the foregoing general description and the following detailed description are exemplary and explanatory only and are not restrictive of the invention claimed. As used herein, the use of the singular includes the plural unless specifically stated otherwise.

The following abbreviations are used in the general schemes and in the examples:
Boc tert-Butyloxycarbonyl
EDCI 1-Ethyl-3-(3-dimethylaminopropyl)carbodiimide
Boc₂O di-tert-butyl dicarbonate
THF tetrahydrofuran
NaOH sodium hydroxide
DMAP 4-dimethylaminopyridine
CH₂Cl₂ dichloromethane
HCl hydrochloric acid
M molar
NaHCO₃ sodium bicarbonate
CHCl3 chloroform
EtOH ethanol
DMF N,N-dimethylformamide
MeOH methanol
NaOAc sodium acetate
FA fumaric acid
PG protecting group

In scheme 1 the synthesis of hybrid compounds were started with a fluoroquinolone (gatifloxacin). The Boc protected gatifloxacin was coupled with a linker in the presence of EDCI. After deprotection a second EDCI coupling with dexmethasone, followed by removal of BOO group and fumaric acid treatment yielded the desired product.

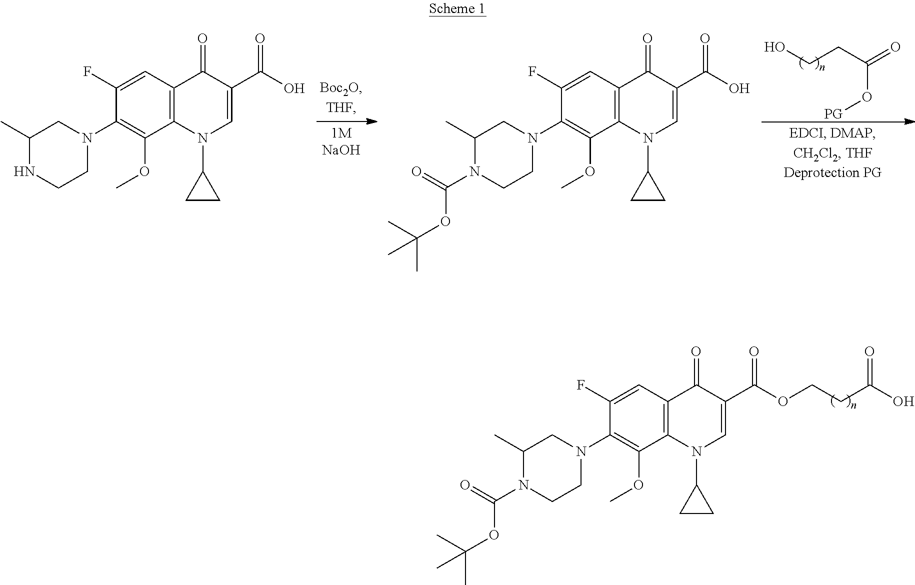

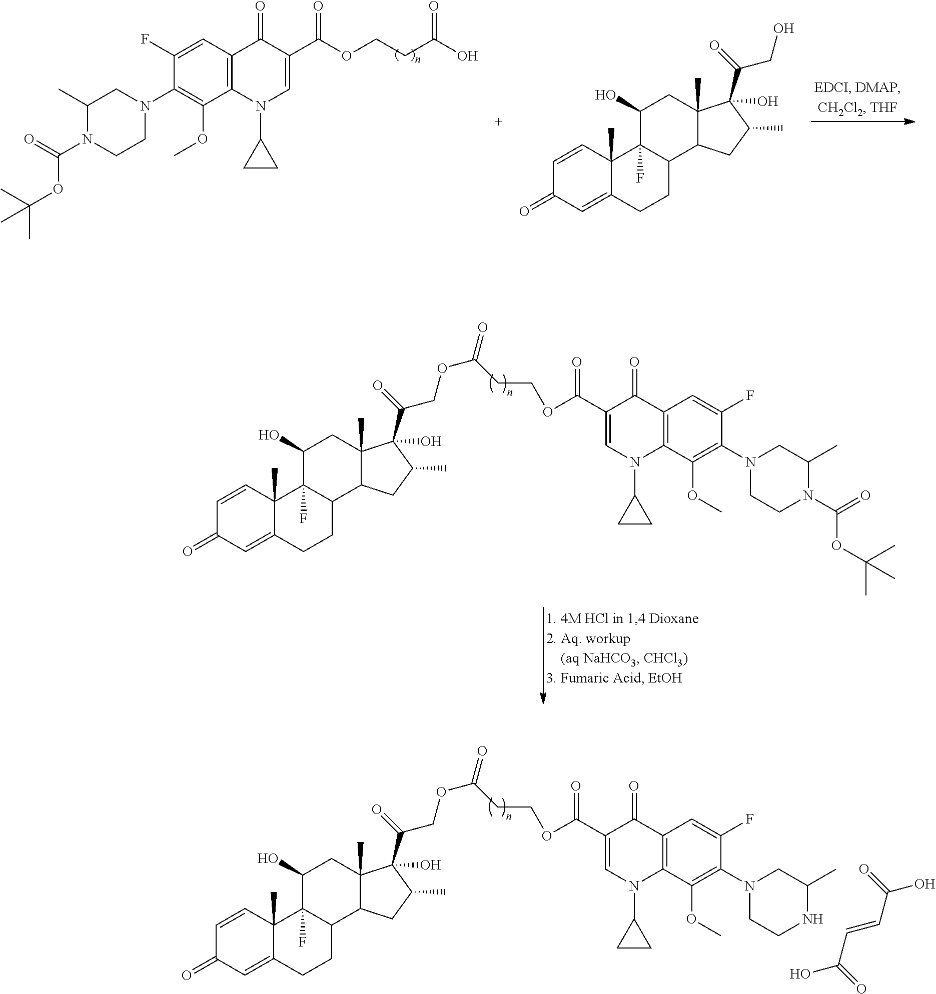

In the following example the synthesis was started by coupling the linker to the steroid.

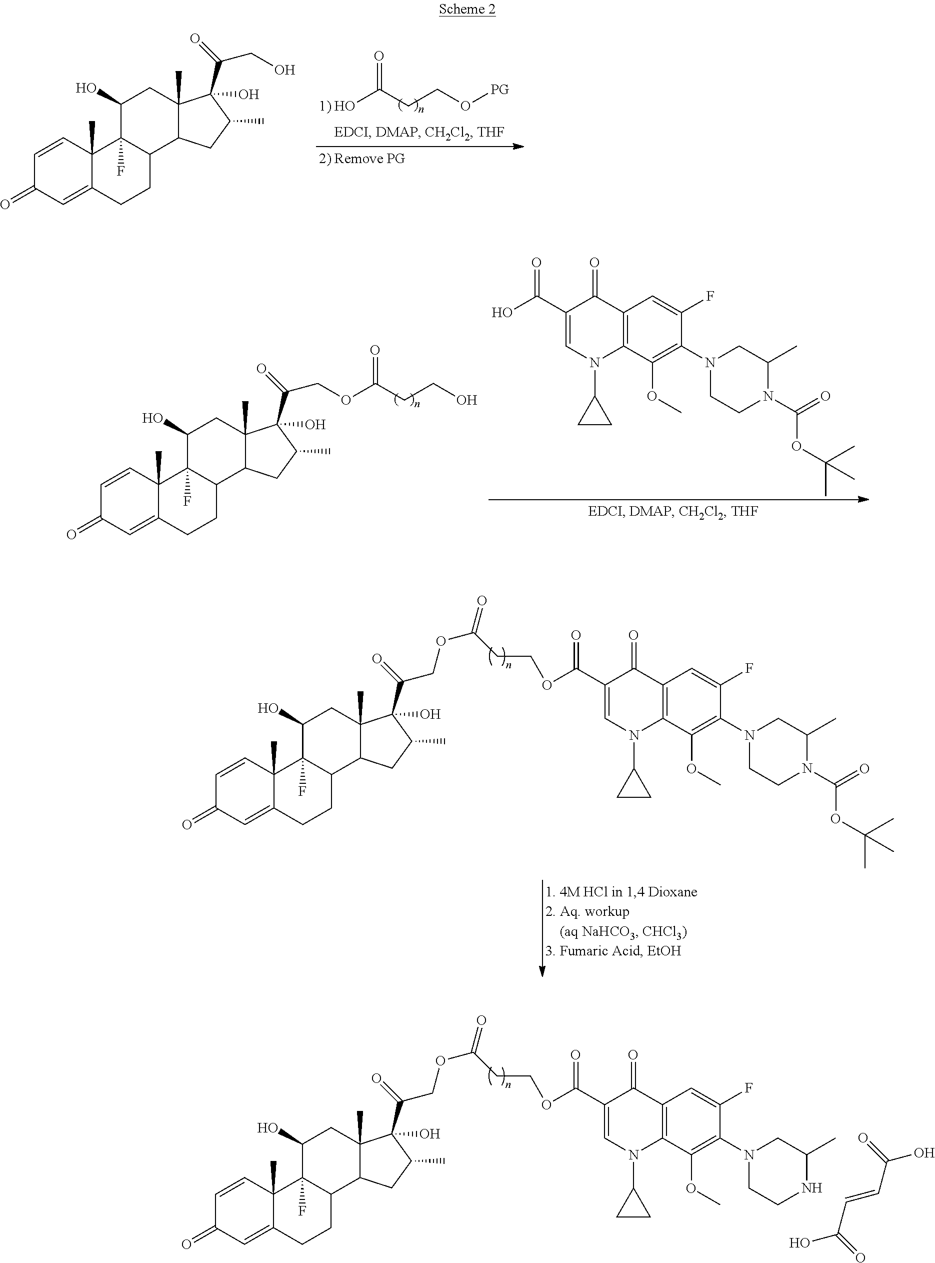

The following example describes the synthesis of Compound 44.

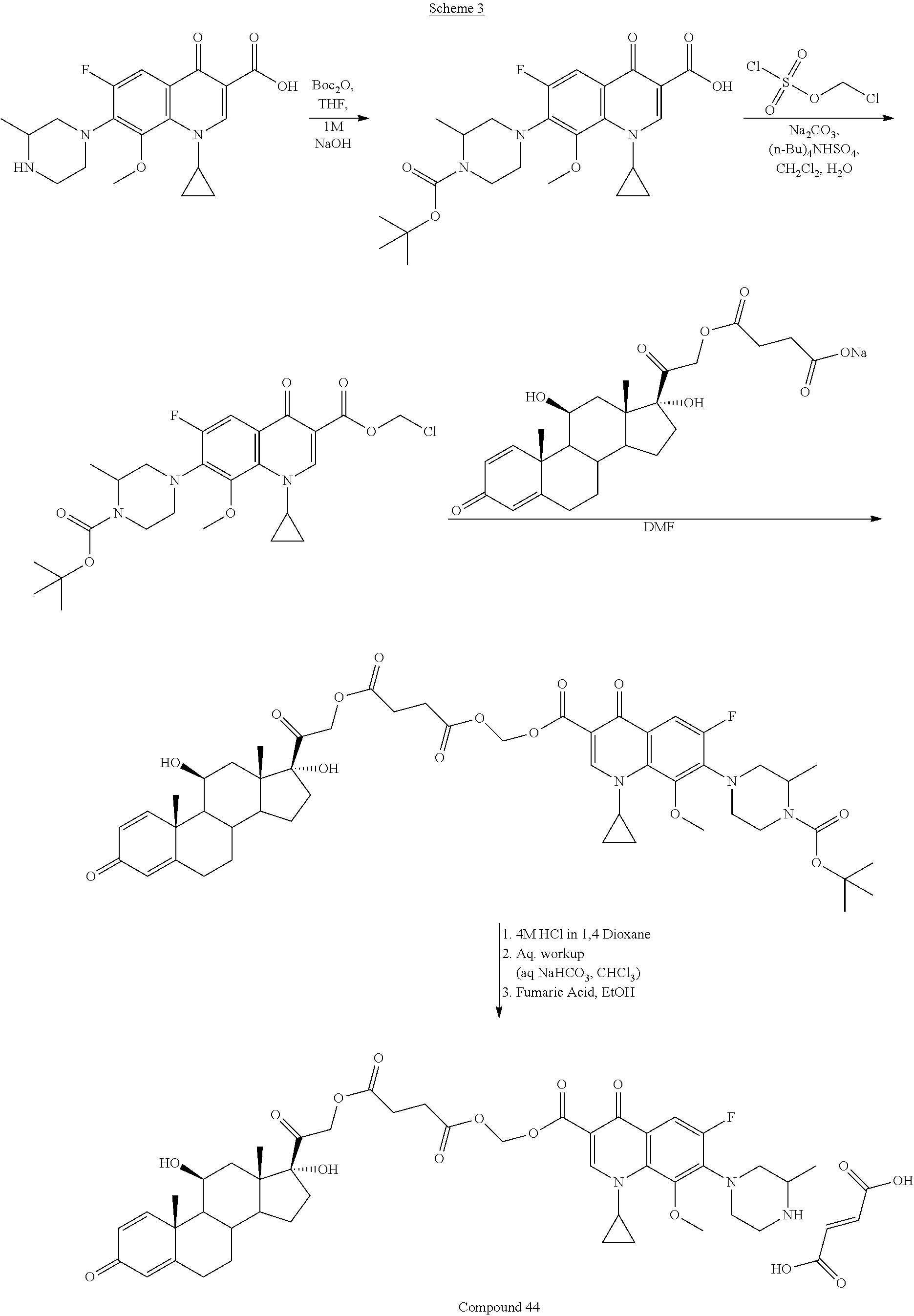

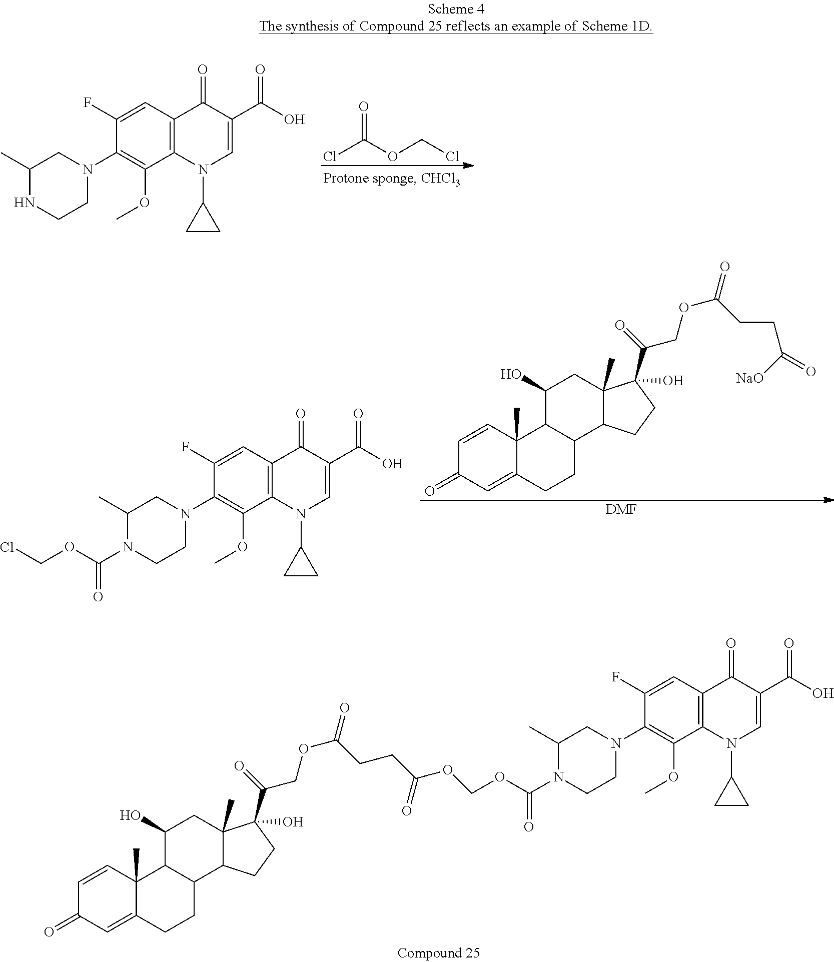

It is to be understood that both the foregoing general description and the following detailed description are exemplary and explanatory only and are not restrictive of the invention claimed. As used herein, the use of the singular includes the plural unless specifically stated otherwise.

It will be readily apparent to those skilled in the art that some of the compounds of the invention may contain one or more asymmetric centers, such that the compounds may exist in enantiomeric as well as in diastereomeric forms. Unless it is specifically noted otherwise, the scope of the present invention includes all enantiomers, diastereomers and racemic mixtures. Some of the compounds of the invention may form salts with pharmaceutically acceptable acids or bases, and such pharmaceutically acceptable salts of the compounds described herein are also within the scope of the invention.

The present invention includes all pharmaceutically acceptable isotopically enriched compounds. Any compound of the invention may contain one or more isotopic atoms enriched or different than the natural ratio such as deuterium ²H (or D) in place of hydrogen ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

The following examples are for illustrative purposes only and are not intended, nor should they be construed as limiting the invention in any manner. Those skilled in the art will appreciate that variations and modifications of the following examples can be made without exceeding the spirit or scope of the invention.

As will be evident to those skilled in the art, individual isomeric forms can be obtained by separation of mixtures thereof in conventional manner. For example, in the case of diasteroisomeric isomers, chromatographic separation may be employed.

Compound names were generated with ACDLabs version 12.5 or ChemBioDraw Ultra version 12.0.2.

In general, characterization of the compounds is performed according to the following methods. Proton nuclear magnetic resonance (¹H NMR) and carbon nuclear magnetic resonance (¹³C NMR) spectra were recorded on a Varian 300 or 600 MHz spectrometer in deuterated solvent. Chemical shifts were reported as δ (delta) values in parts per million (ppm) relative to tetramethylsilane (TMS) as an internal standard (0.00 ppm) and multiplicities were reported as s, singlet; d, doublet; t, triplet; q, quartet; m, multiplet; br, broad. Data were reported in the following format: chemical shift (multiplicity, coupling constant(s) J in hertz (Hz), integrated intensity). The mass spectrometry data were determined on a Shimadzu LCMS-IT-TOF instrument.

The formation of the hybrid compounds was checked by ¹H-NMR by comparing the chemical shifts of protons H^{a}, H^{b} from the antibiotic molecule and of protons H^{c} and/or H^{d} of the steroid molecule with the chemical shifts of these same protons on the newly formed hybrid molecule noted H^{a}*, H^{b}*, H^{c}* and/or H^{d}* wherein “*” indicates the hybrid compound. Applicants have indicated with arrows the location of these protons and the reaction site of the pro-drug moiety, where available. Each scheme shows the formation of the new hybrid drug. Each table describes the results for the new hybrid drug and the linker number, where existing. The linker and pro-drug moiety numbers are as described in Table 1 and 2 respectively.

The following examples are for illustrative purposes only and are not intended, nor should they be construed as limiting the invention in any manner. Those skilled in the art will appreciate that variations and modifications of the following examples can be made without exceeding the spirit or scope of the invention.

### EXAMPLES

Gatifloxacin reacted with betamethasone to form the following hybrid compounds as shown in Scheme 5 with the results described in Table 4 and in Scheme 6 with the results shown in Table 5 and in Scheme 33 with the results shown in Table 34 and in Scheme 37 with the results shown in Table 38.

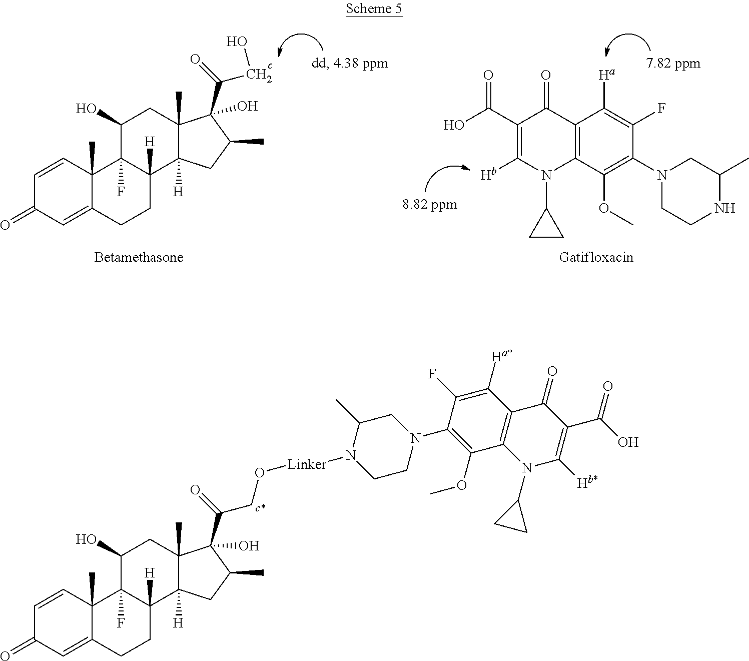

TABLE 4

| * | | | | | |
|---|---|---|---|---|---|
| Compound | | δ ppm | | | |
| No. | Linker | H^{a}* | H^{b}* | CH₂^{c}* | MASS |
| 1 | L4 | 7.85 | 8.86 | 4.98, dd | 872 |
| | | | | | (MNa⁺) |
| 2 | L16 | 7.85 | 8.86 | 4.96 | 946 |
| | | | | | (MNa⁺) |

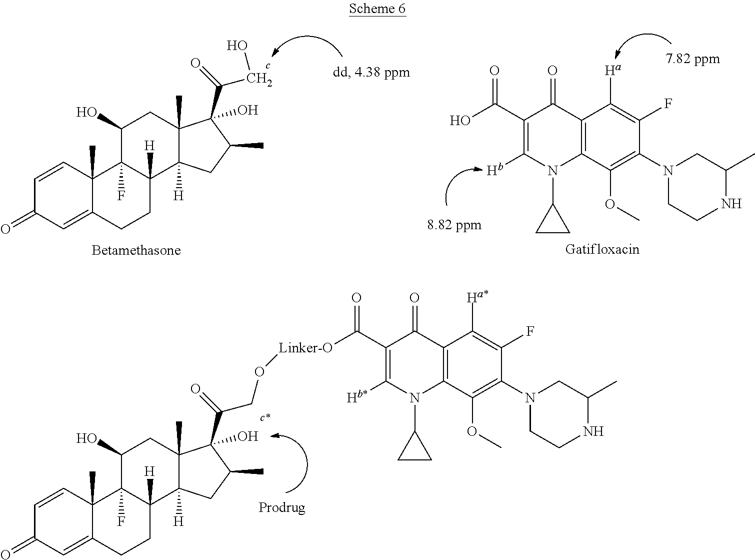

TABLE 5

| * | | | | | |
|---|---|---|---|---|---|
| Compound | | | | | |
| No. | Linker | δ ppm | | | MASS |
| Salt | Prodrug | H^{a}* | H^{b}* | CH₂^{c}* | (MH⁺) |
| HCl | L7 | 7.86 | 8.81 | 4.89 (dd) | 908 |
| 3 | | | | | |
| HCl | L9 | 7.86 | 8.83 | 5.19 (dd) | 884 |
| 4 | | | | | |
| HCl | L8 | 7.86 | 8.86 | 4.91 (dd) | 983 |
| 5 | | | | | |
| 6 | L3 | 7.83 | 8.71 | 4.87 (dd) | 836 |
| HCl | L15 | 7.84 | 8.77 | 4.22 (m) | 894 |
| 7 | | | | | |
| 8 | L19 | 7.83 | 8.77 | 4.86 (m) | 880 |
| 111 | L3 | 7.76 | 8.73 | 4.6 (dd) | |
| | P5 | | | | |
| 120 | L9 | 7.84 | 8.80 | 4.90 (dd) | 926 |
| | P8 | | | | (MH⁺) |

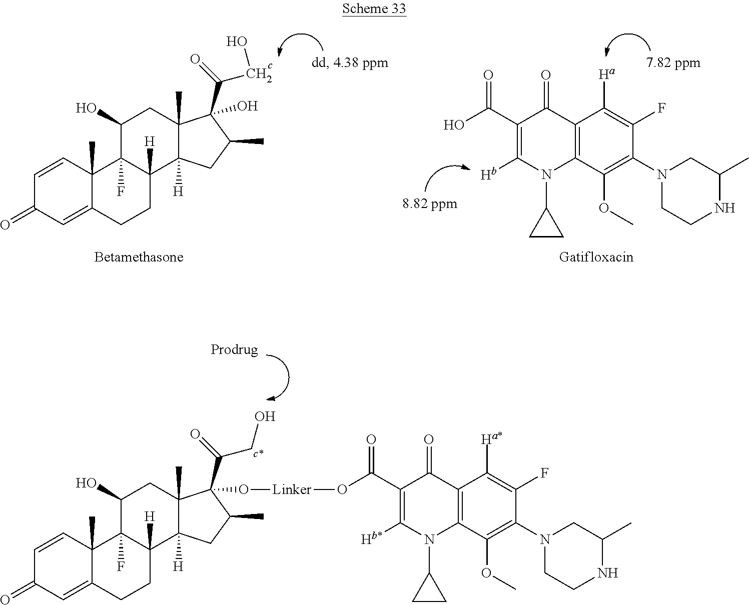

TABLE 34

| * | | | | | |
|---|---|---|---|---|---|
| Compound | Linker | δ ppm | | | MASS |
| No. | Prodrug | H^{a}* | H^{b}* | CH₂^{c}* | (MH⁺) |
| 112 | L3 | 7.77 | 8.73 | 4.64 (dd) | 921 |
| | P6 | | | | (MNa⁺) |
| 113 | L3 | 7.76 | 8.69 | 4.61 (dd) | 907 |
| | P7 | | | | (MH⁺) |
| 125 | L3 | 7.60 | 8.43 | 4.55 | 878 |
| | P8 | (DMSO-d₆) | (DMSO-d₆) | (DMSO-d₆) | (MH⁺) |

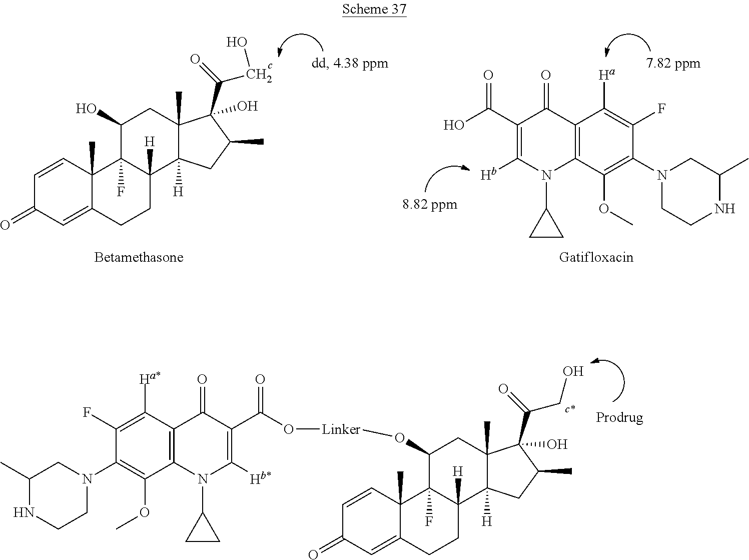

TABLE 38

| * | | | | | |
|---|---|---|---|---|---|
| Compound | Linker | δ ppm | | | MASS |
| No. | Prodrug | H^{a}* | H^{b}* | CH₂^{c}* | (MH⁺) |
| 124 | L5 | 7.83 | 8.79 | 4.84 | 864 |
| | P8 | | | | |

Gatifloxacin reacted with dexmethasone to form the following hybrid compounds as shown in Scheme 7 with the results described in Table 6 and in Scheme 8 with the results shown in Table 7 and Scheme 35 with the results shown in Table 36

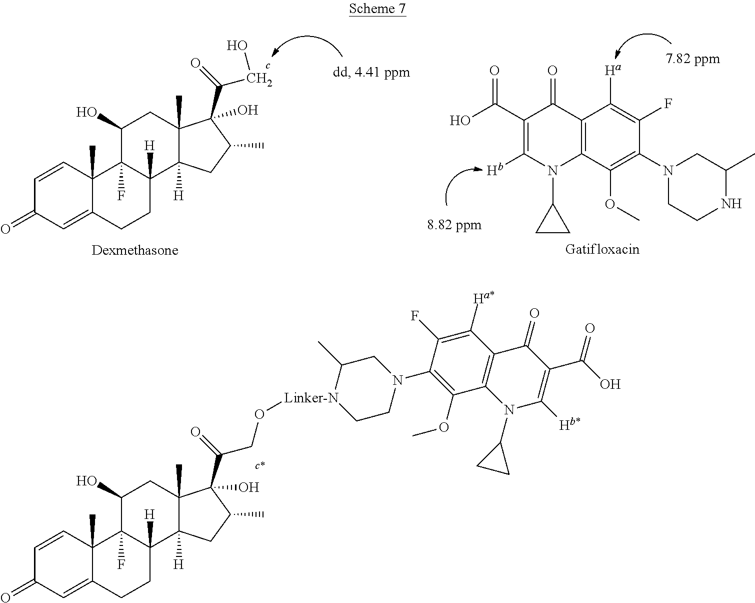

TABLE 6

| * | | | | | |
|---|---|---|---|---|---|
| Compound | | δ ppm | | | |
| No. | Linker | H^{a}* | H^{b}* | CH₂^{c}* | MASS |
| 9 | L1 | 7.81 | 8.85 | 5.04 (m) | 808 (MH⁺) |
| 10 | L4 | 7.84 | 8.87 | 4.99 (dd) | 872 (MH⁺) |
| 11 | L16 | 7.84 | 8.86 | 4.97 (dd) | 946 (MNa⁺) |
| 12 | L24 | 7.82 | 8.85 | 5.23 (dd) | 970 (MH⁻) |
| 13 | L39 | 7.81 | 8.85 | 4.97 (dd) | 1045 (MNa⁺) |
| 14 | L40 | 7.83 | 8.86 | 4.97 (dd) | |
| 88 | L34 | 7.89 | 8.81 | 5.01 | 962 (MNa⁺) |

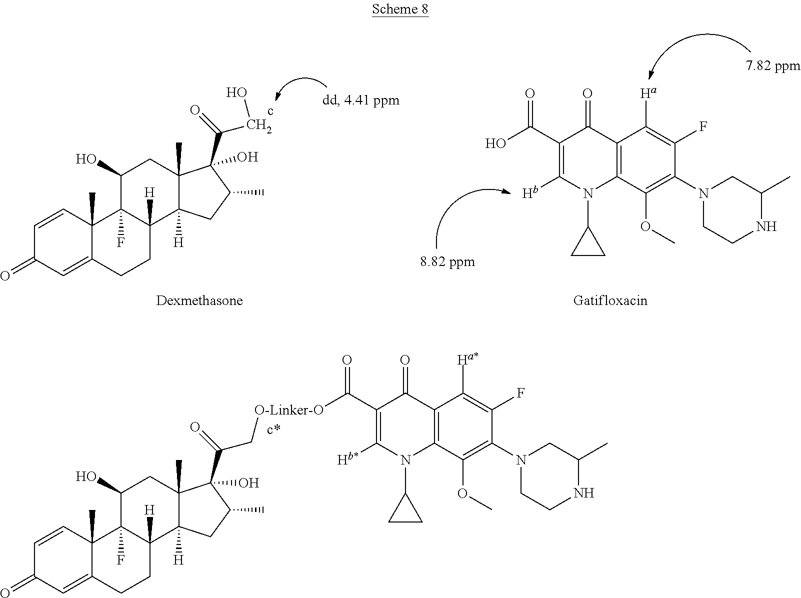

TABLE 7

| * | | | | | |
|---|---|---|---|---|---|
| Com- | | | | | |
| pound | | | | | |
| No. | | δ ppm | | | |
| Salt | Linker | H^{a}* | H^{b}* | CH₂^{c}* | MASS |
| 16 | L1 | 7.86 | 8.94 | 5.07 (dd) | 808 (MH⁺) |
| 18 | L7 | 8.01 | 8.86 | 5.06 (dd) | 906 (MH⁻) |
| HCl | L8 | 7.64 | 8.48 | 4.89 (dd) | 982 (MH⁺) |
| 19 | | (DMSO-d₆) | (DMSO-d₆) | (DMSO-d₆) | |
| 20 | L25 | 7.80 | 8.72 | 5.22 (dd) | 942 (MH⁻) |
| 21 | L11 | 7.81 | 8.74 | 5.08 (dd) | 890 (MH⁻) |
| 22 | L9 | 7.86 | 8.90 | 5.18 (dd) | 884 (MH⁺) |
| 23 | L3 | 7.84 | 8.72 | 4.86 (dd) | 834 (MH⁻) |
| 24 | L19 | 7.54 | 8.73 | 4.90 (dd) | 850 (MH⁺) |
| 90 | L28 | 7.82 | 8.83 | 5.25 (dd) | 928 (MH⁺) |

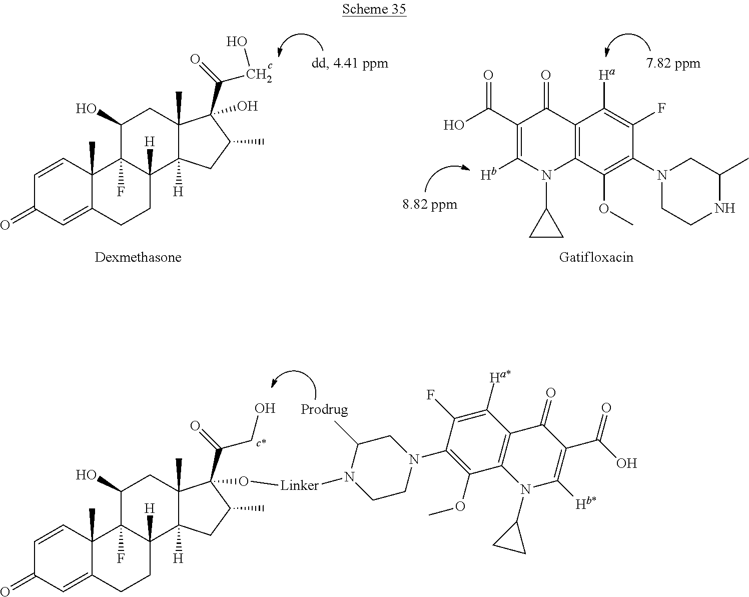

TABLE 36

| * | | | | | |
|---|---|---|---|---|---|
| Compound | Linker | δ ppm | | | |
| No. | Prodrug | H^{a}* | H^{b}* | CH₂^{c}* | MASS |
| 117 | L3 | 7.83 | 8.74 | 4.92 (dd) | 876 (MH⁻) |
| | P8 | | | | |

Gatifloxacin reacted with predisolone to form the following hybrid compounds as shown in Scheme 9 with the results described in Table 8 and in Scheme 10 with the results shown in Table 9 and in Scheme 34 with the results in Table 35 and in Scheme 36 with the results in Table 37; and in Scheme 39 with the results in Table 39.

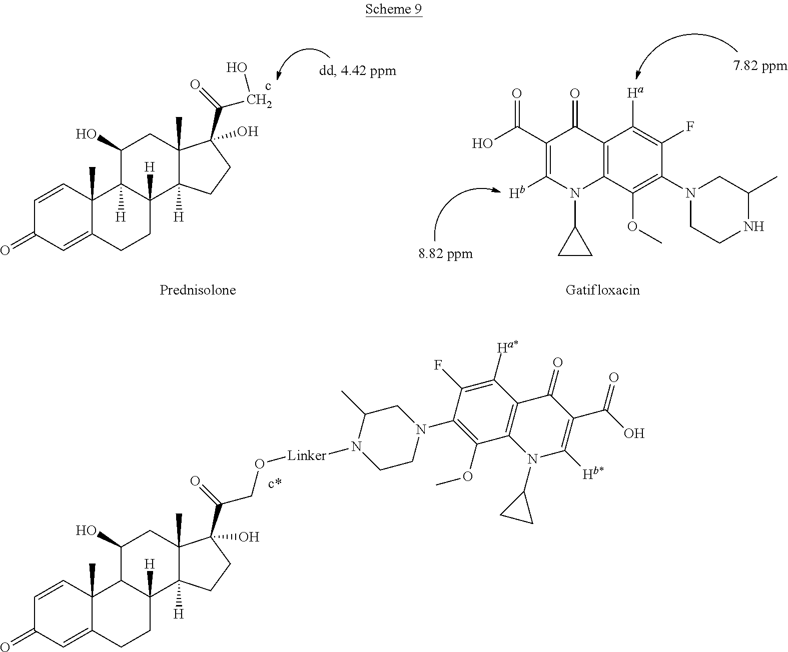

TABLE 8

| * | | | | | |
|---|---|---|---|---|---|
| Compound | | δ ppm | | | |
| No. | Linker | H^{a}* | H^{b}* | CH₂^{c}* | MASS |
| 25 | L16 | 7.85 | 8.87 | 4.99 (dd) | 814 (MNa⁺) |
| 26 | L21 | 7.83 | 8.86 | 4.98 (dd) | 928 (MNa⁺) |
| 27 | L24 | 7.83 | 8.86 | 5.25 (dd) | |
| 28 | L42 | 7.82 | 8.90 | 5.25-4.99 | 907 (MH⁺) |
| 29 | L39 | 7.82 | 8.85 | 4.99 | 1013 (MNa⁺) |
| 30 | L40 | 7.82 | 8.86 | 4.96 (dd) | 1029 (MNa⁺) |
| 31 | L41 | 7.82 | 8.86 | 4.96 (dd) | 1001 (MNa⁺) |
| 32 | L38 | 7.83 | 8.90 | 5.09 | 907 (MH⁺) |
| 33 | L30 | 7.83 | 8.86 | 4.96 (dd) | 928 (MNa⁺) |
| 34 | L44 | 7.82 | 8.89 | 5.07-4.89 | |
| 35 | L32 | 7.78 | 8.84 | 5.07 (dd) | 921 (MH⁺) |
| 36 | L38 | 7.85 | 8.89 | 5.21-4.89 | 855 (MNa⁺) |
| 37 | L43 | 7.84 | 8.91 | 5.19 | 847 (MH⁺) |
| 38 | L42 | 7.83 | 8.92 | 5.18 (dd) | 843 (MNa⁺) |
| 39 | L37 | 7.78 | 8.84 | 5.00 (dd) | 854 (MNa⁺) |
| 92 | L34 | 7.82 | 8.82 | 5.05 (dd) | 930 (MNa⁺) |
| 96 | L36 | 7.87 | 8.80 | 5.14 (dd) | 944 (MNa⁺) |
| 97 | L48 | 7.81 | 8.86 | 5.05 (dd) | 869 (MNa⁺) |
| 99 | L49 | 7.80 | 8.85 | 4.98 (dd) | 1043 (MNa⁺) |
| 100 | L50 | 7.81 | 8.86 | 5.19 (dd) | |
| 101 | L51 | 7.78 | 8.84 | 5.03 (dd) | 1029 (MNa⁺) |
| 102 | L52 | 7.81 | 8.85 | 5.00 (dd) | 1101 (MNa⁺) |
| 104 | L53 | 7.82 | 8.86 | 5.01 (dd) | |

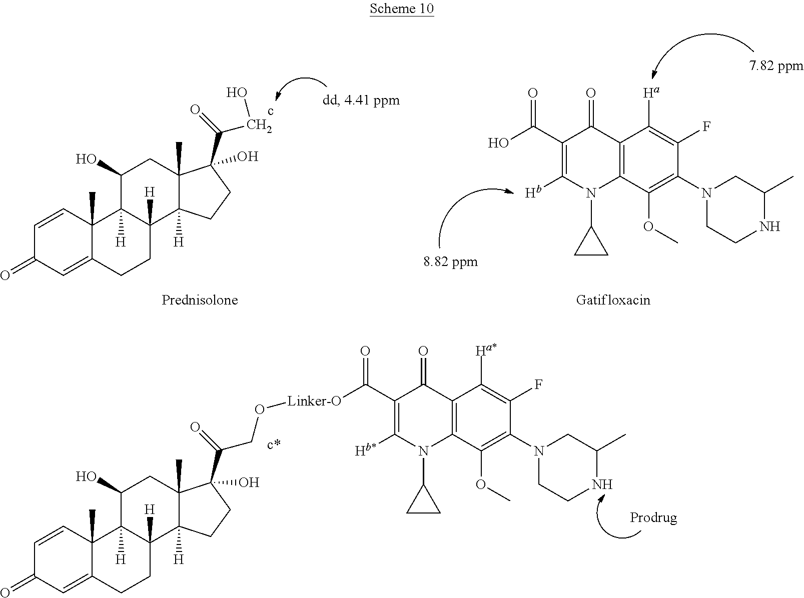

TABLE 9

| * | | | | | | | |
|---|---|---|---|---|---|---|---|
| Compound | Linker | δ ppm | | | | | |
| No. | Prodrug | H^{a}* | H^{b}* | CH₂^{c}* | | MASS | |
| 41 | L3 | 7.82 | 8.74 | 4.91 | (dd) | | |
| 42 | L17 | 7.78 | 8.82 | 5.03 | (dd) | 792 | (MH⁺) |
| 43 | L18 | 7.80 | 8.81 | 5.01 | (m) | 807 | (MH⁺) |
| 44 | L19 | 7.82 | 8.78 | 4.94 | (dd) | 848 | (MH⁺) |
| 45 | L28 | 7.82 | 8.83 | 5.25 | | | |
| 46 | L29 | 7.76 | 8.77 | 4.93 | (dd) | 862 | (MH⁺) |
| 94 | L35 | 7.76 | 8.78 | 4.96 | (dd) | 900 | (MNa⁺) |
| 98 | L31 | 7.80 | 8.79 | 5.05 | (dd) | 899 | (MNa⁺) |
| 107 | L19 | 7.72 | 8.74 | 4.88 | (dd) | 982 | (MNa⁺) |
| | P3 | | | | | | |
| 108 | L8 | 7.75 | 8.75 | 4.92 | (dd) | 1084 | (MNa⁺) |
| | P3 | | | | | | |

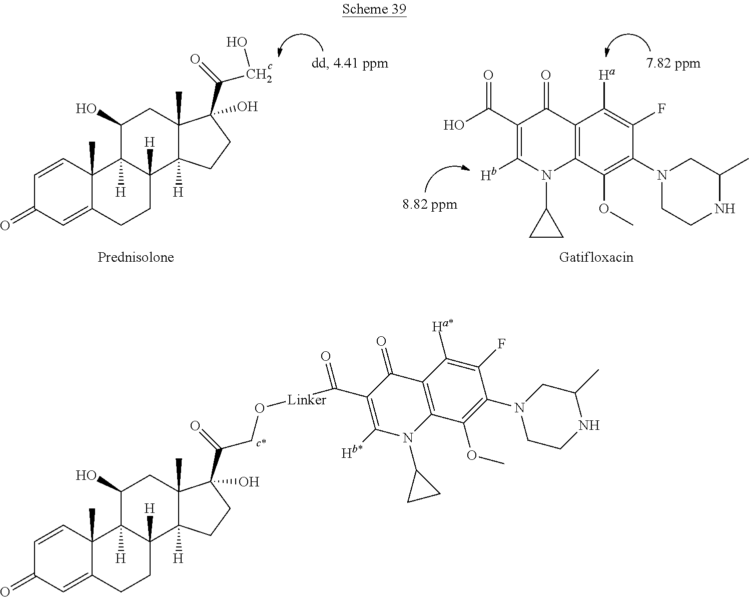

TABLE 39

| * | | | | | | | |
|---|---|---|---|---|---|---|---|
| Compound | | δ ppm | | | | | |
| No. | Linker | H^{a}* | H^{b}* | CH₂^{c}* | | MASS | |
| 40 | L6 | 7.87 | 8.86 | 4.90 | (dd) | 825 | (MNa⁺) |
| 17 | L5 | 7.86 | 8.92 | 5.0 | | 821 | (MH⁺) |

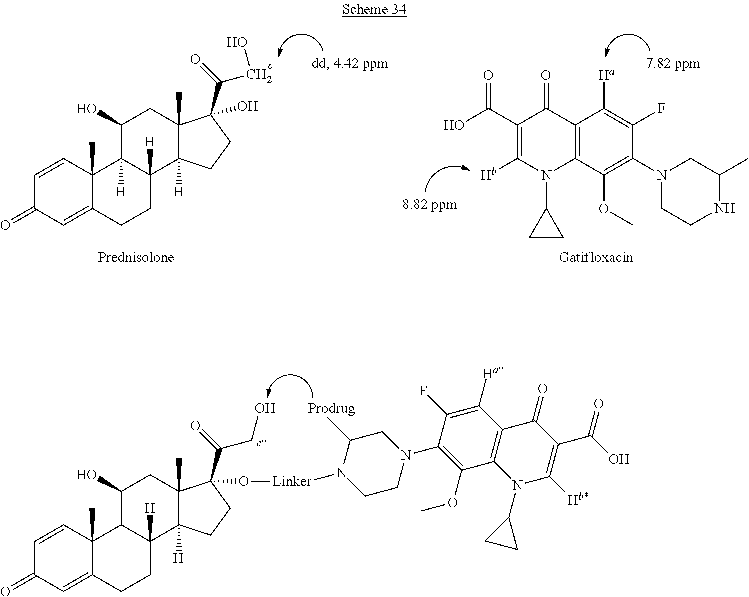

TABLE 35

| * | | | | | | |
|---|---|---|---|---|---|---|
| Compound | Linker | δ ppm | | | | |
| No. | Prodrug | H^{a}* | H^{b}* | CH₂^{c}* | MASS | |
| 115 | L3 | 7.77 | 8.70 | 4.83 (dd) | 886 | (MNa⁺) |
| | P6 | | | | | |
| 116 | L3 | 7.53 | 8.71 | 4.83 (dd) | 931 | (MH⁻) |
| | P8 | | | | | |
| 123 | L3 | 7.89 | 8.90 | 4.85 (dd) | 874 | (MNa⁺) |
| | P7 | | | | | |

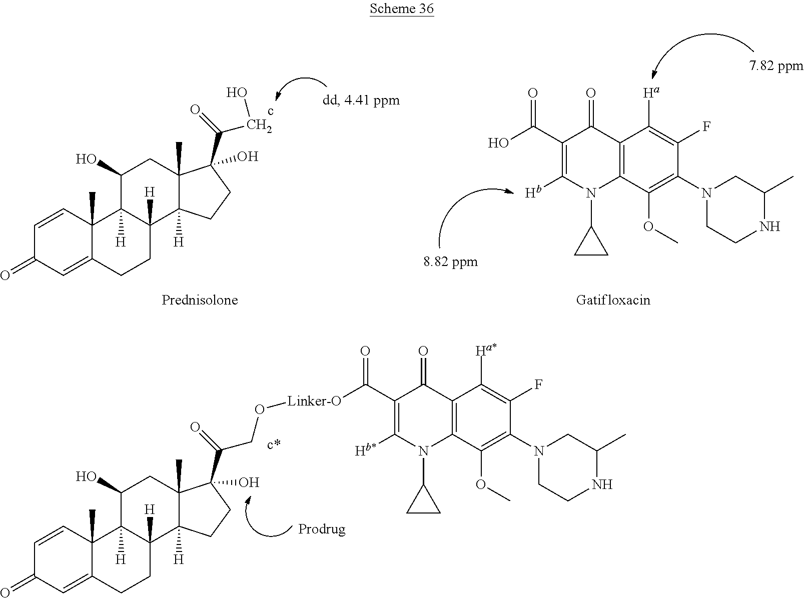

TABLE 37

| * | | | | | |
|---|---|---|---|---|---|
| Compound | Linker | δ ppm | | | |
| No. | Prodrug | H^{a}* | H^{b}* | CH₂^{c}* | MASS |
| 118 | L3 | 7.81 | 8.874 | 4.88 (dd) | 908 (MH⁻) |
| | P9 | | | | |
| 119 | L3 | 7.96 | 9.03 | 4.82 (dd) | 874 (MH⁻) |
| | P5 | | | | |

Gatifloxacin reacted with hydrocortisone to form the following hybrid compounds as shown in Scheme 11 with the results described in Table 10 and in Scheme 12 with the results shown in Table 11.

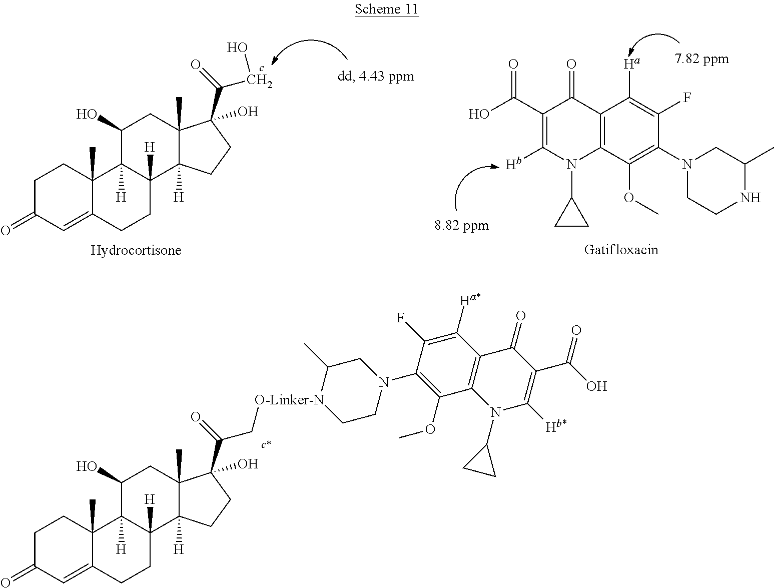

TABLE 10

| * | | | | |
|---|---|---|---|---|
| Compound | | δ ppm | | |
| No. | Linker | H^{a}* | H^{b}* | CH₂^{c}* |
| 47 | L16 | 7.85 | 8.87 | 5.0 |

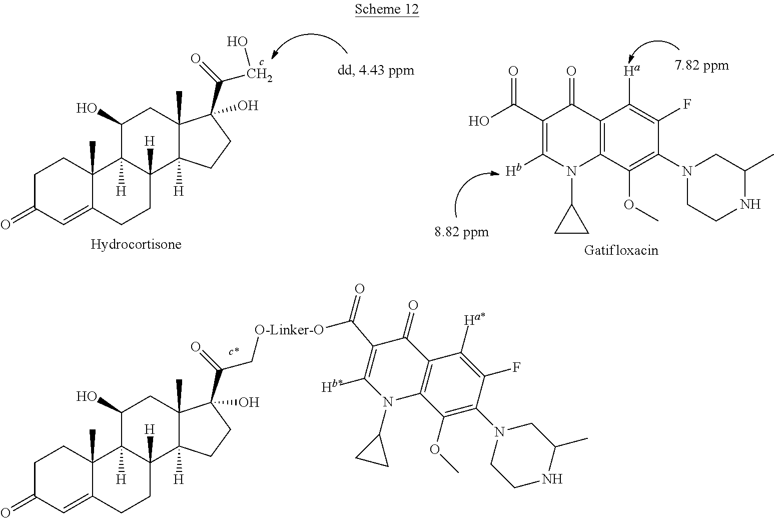

TABLE 11

| * | | | | | |
|---|---|---|---|---|---|
| Compound | | δ ppm | | | |
| No. | Linker | H^{a}* | H^{b}* | CH₂^{c}* | MASS |
| 48 | L19 | 7.80 | 8.78 | 4.95 (dd) | 850 (MH⁺) |

Moxifloxacin reacted with betamethasone to form the following hybrid compounds as shown in Scheme 13 with the results described in Table 12 and in Scheme 14 with the results shown in Table 13.

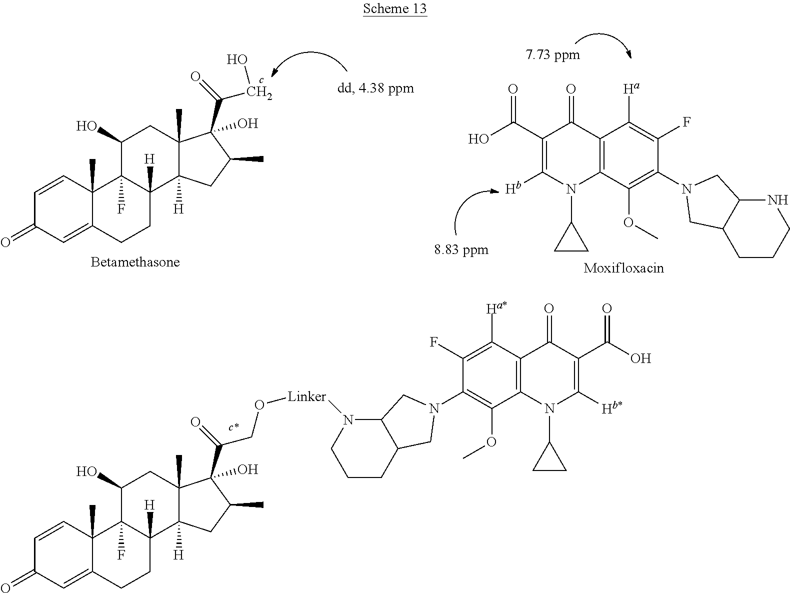

TABLE 12

| * | | | | | |
|---|---|---|---|---|---|
| Compound | | δ ppm | | | |
| No. | Linker | H^{a}* | H^{b}* | CH₂^{c}* | MASS |
| 49 | L16 | 7.80 | 8.77 | 4.95 (brs, m) | 972 (MH⁺) |

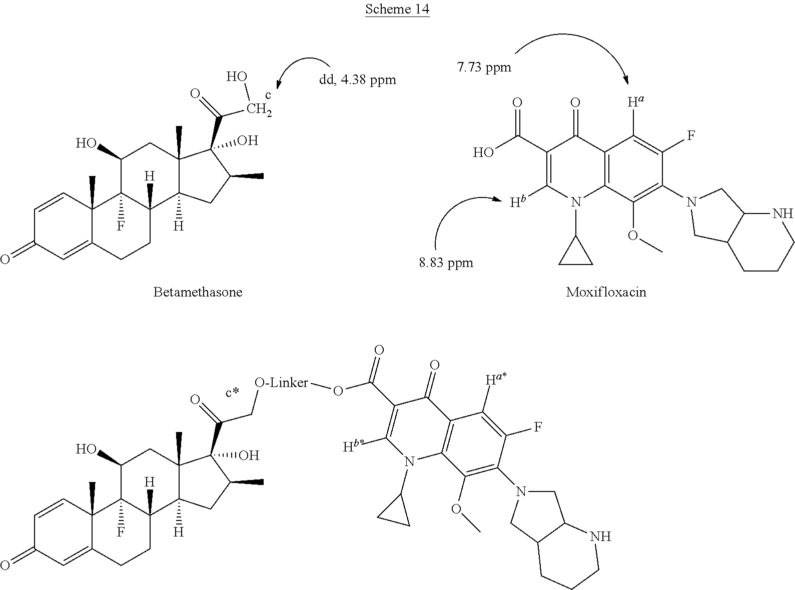

TABLE 13

| * | | | | |
|---|---|---|---|---|
| Compound | | δ ppm | | |
| No. | Linker | H^{a}* | H^{b}* | CH₂^{c}* |
| 50 | L19 | 7.62 | 8.73 | 4.83 |

Moxifloxacin reacted with dexmethasone to form the following hybrid compounds as shown in Scheme 15 with the results described in Table 14 and in Scheme 16 with the results shown in Table 15.

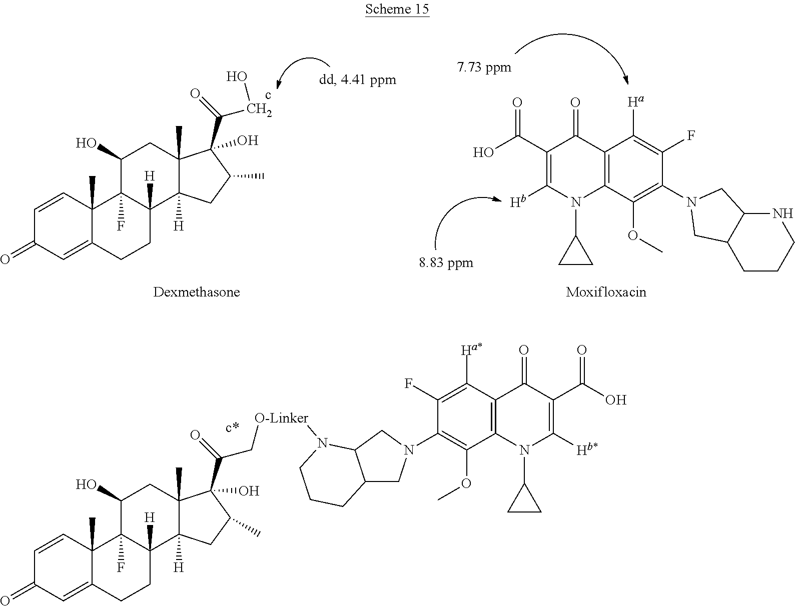

TABLE 14

| * | | | | | | |
|---|---|---|---|---|---|---|
| Compound | | δ ppm | | | | |
| No. | Linker | H^{a}* | H^{b}* | CH₂^{c}* | | MASS |
| 51 | L16 | 7.74 | 8.81 | 4.89 | (dd) | 972 (MNa⁺) |
| 52 | L24 | 7.68 | 8.80 | 5.23 | (dd) | 1021 (MNa⁺) |
| 53 | L39 | 7.71 | 8.80 | 4.97 | | 1071 (MNa⁺) |
| 87 | L34 | 7.82 | 8.78 | 4.98 | (dd) | |
| 103 | L39 | 7.71 | 8.81 | 4.96 | (dd) | 1071 (MNa⁺) |

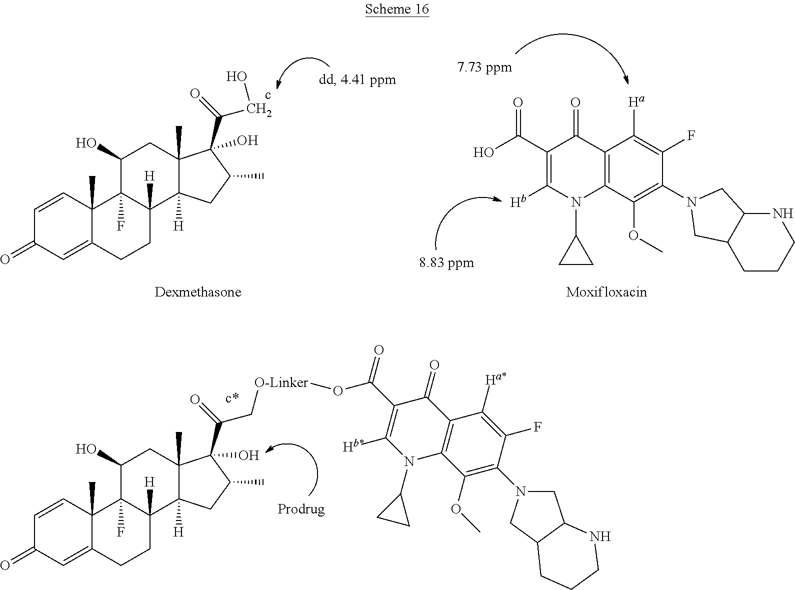

TABLE 15

| * | | | | | | |
|---|---|---|---|---|---|---|
| Compound | | | | | | |
| No. | | δ ppm | | | | |
| Salt | Linker | H^{a}* | H^{b}* | CH₂^{c}* | | MASS |
| 54 | L26 | 7.49 | 8.60 | 5.20 | (dd) | 982 (MH⁺) |
| 55 | L27 | 7.56 | 8.70 | 5.02 | (dd) | 966 (MH⁻) |
| HCl | L7 | 7.89 | 9.13 | 4.94 | (dd) | 935 (MH⁻) |
| 56 | | | | | | |
| 57 | L3 | 7.53 | 8.68 | 4.9 | (dd) | 896 (MH⁻) |
| 58 | L19 | 7.53 | 8.73 | 4.9 | (dd) | 906 (MH⁺) |
| 114 | L3 | 7.71 | 8.71 | 4.83 | | 886 (MH⁻) |
| | P5 | | | | | |

Moxifloxacin reacted with prednisolone to form the following hybrid compounds as shown in Scheme 17 with the results described in Table 16 and in Scheme 18 with the results shown in Table 17.

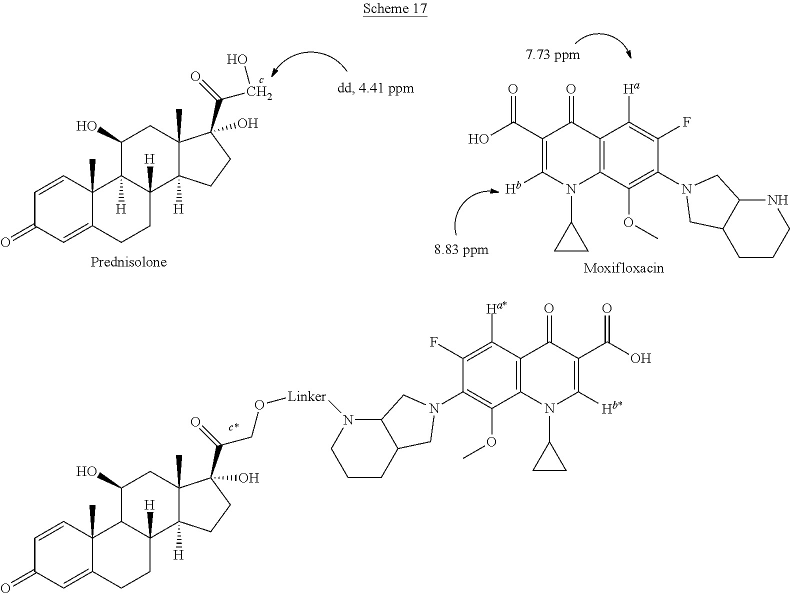

TABLE 16

| * | | | | | | | |
|---|---|---|---|---|---|---|---|
| Compound | | δ ppm | | | | | |
| No. | Linker | H^{a}* | H^{b}* | CH₂^{c}* | | MASS | |
| 59 | L16 | 7.73 | 8.80 | 4.92 | (dd) | 940 | (MNa⁺) |
| 60 | L24 | 7.68 | 8.79 | 5.25 | (dd) | | |
| 61 | L39 | 7.72 | 8.81 | 4.98 | | 1039 | (MNa⁺) |
| 89 | L40 | 7.72 | 8.82 | 4.99 | (dd) | 1057 | (MNa⁺) |
| 91 | L34 | 7.72 | 8.81 | 5.03 | (dd) | 956 | (MNa⁺) |
| 95 | L36 | 7.71 | 8.76 | 4.99 | (dd) | 970 | (MNa⁺) |

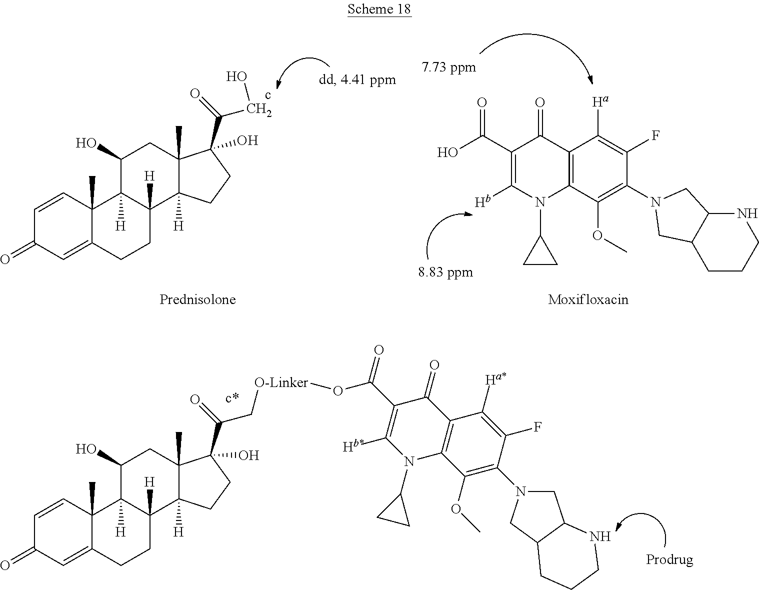

TABLE 17

| * | | | | | | |
|---|---|---|---|---|---|---|
| Compound | | | | | | |
| No. | Linker | δ ppm | | | | |
| Salt | Prodrug | H^{a}* | H^{b}* | CH₂^{c}* | | MASS |
| 63 | L3 | 7.54 | 8.69 | 4.95 | (dd) | 728 (MH⁻) |
| 64 | L17 | 7.46 | 8.78 | 5.05 | (dd) | 818 (MH⁺) |
| 65 | L18 | 7.46 | 8.74 | 5.05 | (m) | 832 (MH⁺) |
| 66 | L19 | 7.62 | 8.74 | 4.94 | (dd) | 874 (MH⁺) |
| 67 | L28 | 7.69 | 8.79 | 5.25 | (dd) | |
| 106 | L19 | 7.70 | 8.73 | 4.92 | (dd) | 988 (MH⁺) |
| | P3 | | | | | |

Moxifloxacin reacted with hydrocortisone to form the following hybrid compounds as shown in Scheme 19 with the results described in Table 18 and in Scheme 20 with the results shown in Table 19, and as shown in Scheme 38 with the results presented in Table 34.

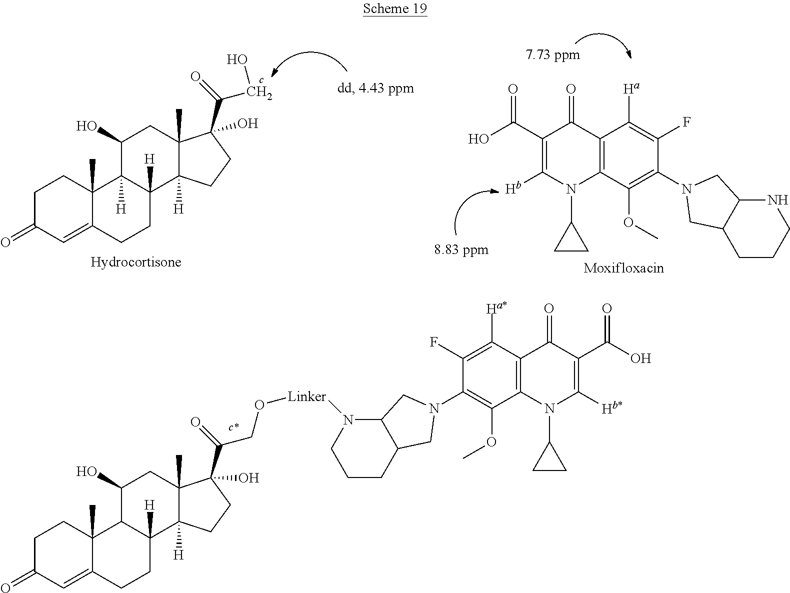

TABLE 18

| * | | | | | |
|---|---|---|---|---|---|
| Compound | | | | | |
| No. | | δ ppm | | | |
| Salt | Linker | H^{a}* | H^{b}* | CH₂^{c}* | MASS |
| 68 | L16 | 7.81 | 8.78 | 4.95 (brs, m) | 942 (MH⁺) |

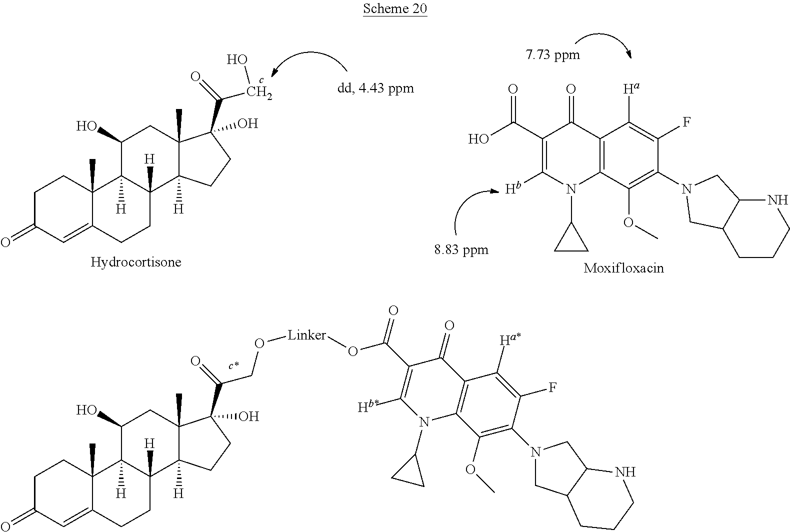

TABLE 19

| * | | | | | |
|---|---|---|---|---|---|
| Compound | | δ ppm | | | |
| No. | Linker | H^{a}* | H^{b}* | CH₂^{c}* | MASS |
| 69 | L19 | 7.65 | 8.71 | 4.94 (dd) | 876 (MH⁺) |

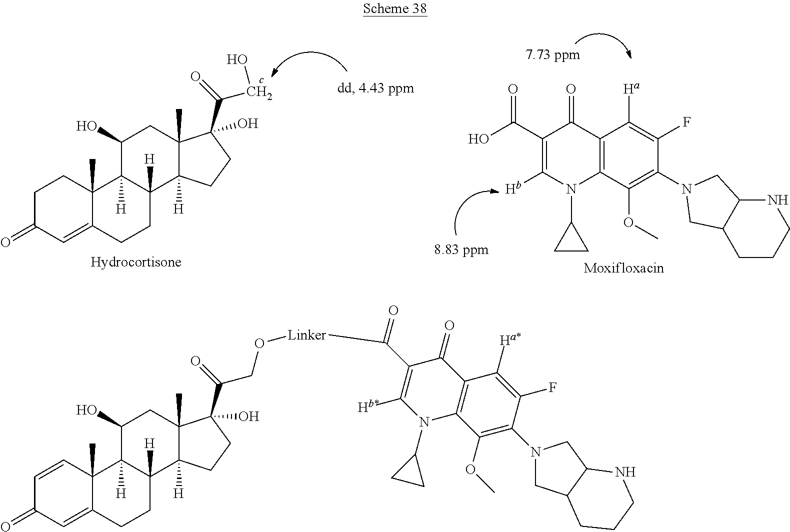

TABLE 34

| * | | | | | |
|---|---|---|---|---|---|
| Compound | | | | | |
| No. | Linker | δ ppm | | | |
| Salt | Prodrug | H^{a}* | H^{b}* | CH₂^{c}* | MASS |
| HCl | L6 | 7.63 | 8.60 | 4.88 (dd) | 829 (MH⁺) |
| 62 | | | | | |

Dexmathsone reacted with besifloxacin to form the following hybrid compounds as shown in Scheme 21 with the results described in Table 20

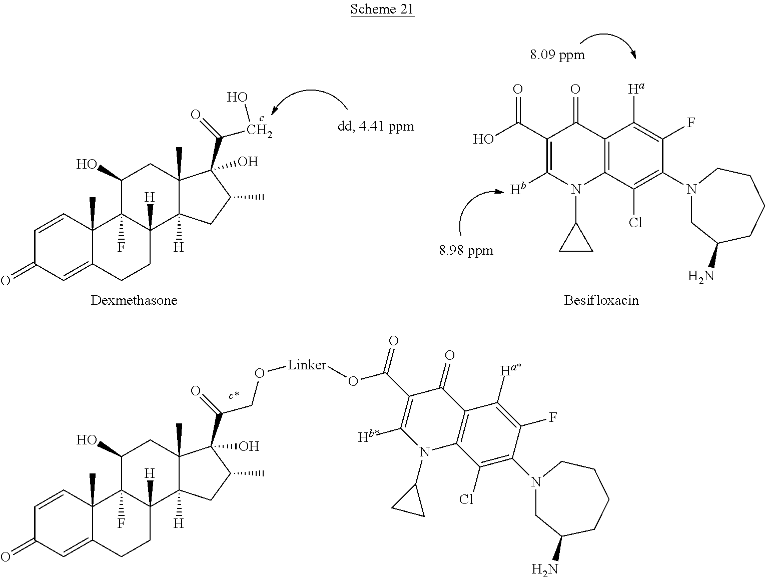

TABLE 20

| *Compound | | δ ppm | | |
|---|---|---|---|---|
| No. | Linker | H^{a}* | H^{b}* | CH₂^{c}* |
| 70 | L19 | 7.99 | 8.89 (300 MHz) | 4.92 (dd) |

Besifloxacin reacted with prednisolone to form the following hybrid compounds as shown in Scheme 22 with the results described in Table 21 and shown in Scheme 32 with results described in Table 33.

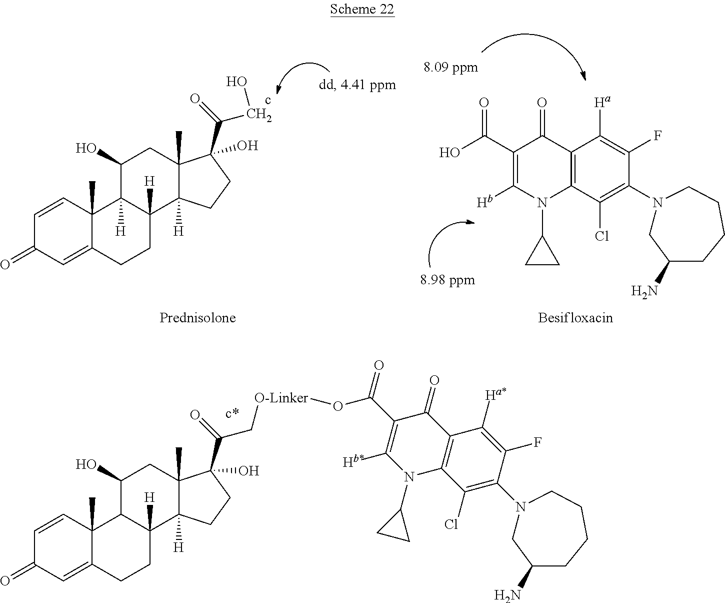

TABLE 21

| | |
|---|---|
| *Compound | δ ppm |

| No. | Linker | H^{a}* | H^{b}* | CH₂^{c}* |
|---|---|---|---|---|
| 71 | L19 | 7.93 | 8.88 | 4.91 (dd) |

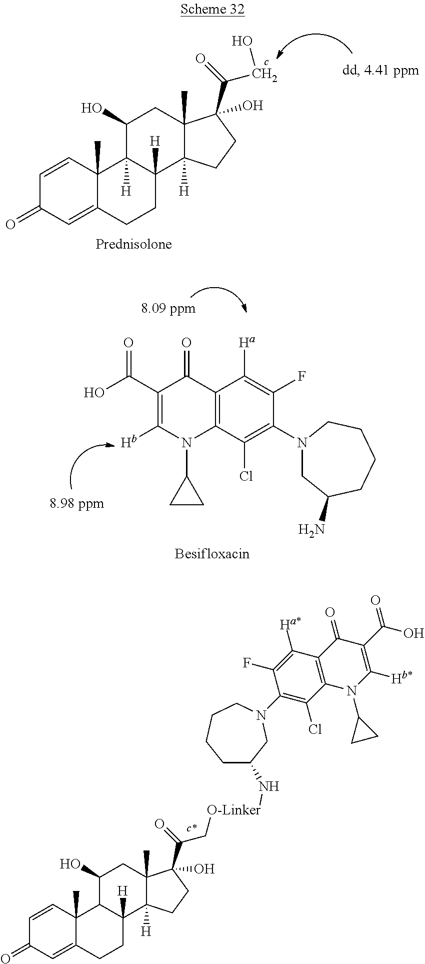

TABLE 33

| *Compound | | δ ppm | | | |
|---|---|---|---|---|---|
| No. | Linker | H^{a}* | H^{b}* | CH₂^{c}* | MASS |
| 110 | L16 | 8.03 | 8.97 | 5.01 (dd) | 911 MH+ |

Hydrocortisone reacted with besifloxacin to form the following hybrid compounds as shown in Scheme 23 with the results shown in Table 22.

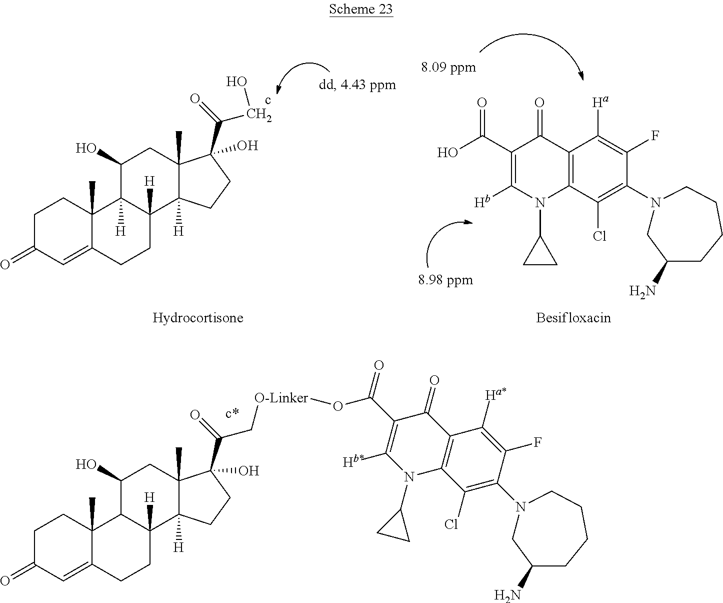

TABLE 22

| *Compound | | δ ppm | | | |
|---|---|---|---|---|---|
| No. | Linker | H^{a}* | H^{b}* | CH₂^{c}* | MASS |
| 72 | L19 | 7.94 | 8.88 | 4.96 | 868 (MH⁺) |

Chloramphenicol reacted with betamethasone to form the following hybrid compounds as shown in Scheme 24 with the results shown in Table 23.

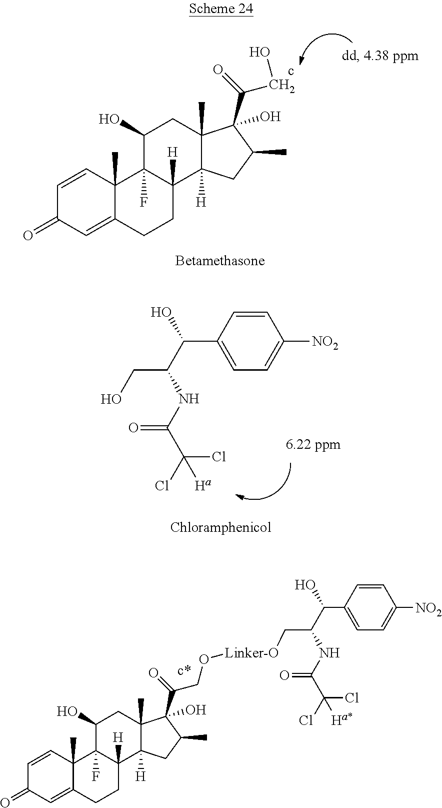

TABLE 23

| *Compound | | δ ppm | | | |
|---|---|---|---|---|---|
| No. | Linker | H^{a}* | CH₂^{c}* | | MASS |
| 73 | L4 | 6.20 | 4.96, | dd | 819 (MNa⁺) |
| 126 | L4 | 6.22 | 4.99 | dd | 820 (MNa+) |

Chloramphenicol reacted with dexmethasone to form the following hybrid compounds as shown in Scheme 25 with the results shown in Table 24.

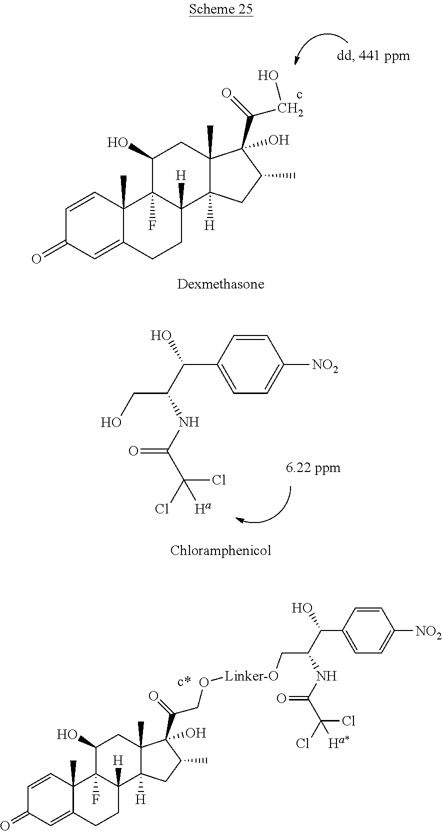

TABLE 24

| | |
|---|---|
| *Compound | δ ppm |

| No. | Linker | H^{a}* | CH₂^{c}* | MASS |
|---|---|---|---|---|
| 74 | L12 | 6.22 | 5.23, dd | 867 (MH⁺) |

Chloramphenicol reacted with prednisolone to form the following hybrid compounds as shown in Scheme 26 with the results shown in Table 25.

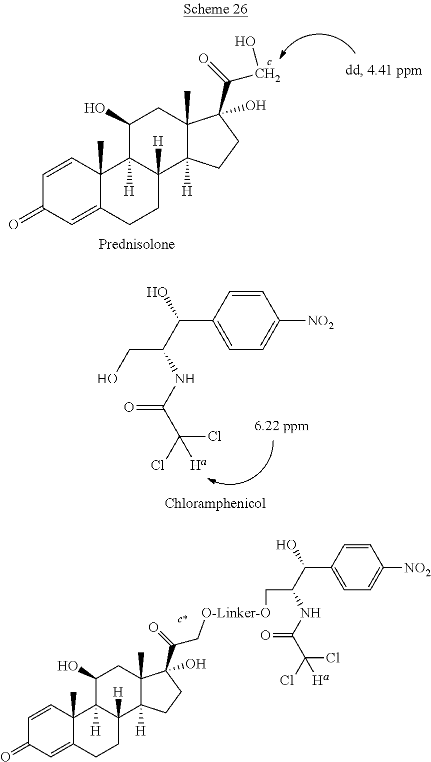

TABLE 25

| | |
|---|---|
| *Compound | δ ppm |

| No. | Linker | H^{a}* | CH₂^{c}* | MASS |
|---|---|---|---|---|
| 75 | L4 | 6.25 | 5.02 (dd) | 787 (MNa⁺) |

Tobramycin reacted with betamethasone to form the following hybrid compounds as shown in Scheme 27 with the results shown in Table 26.

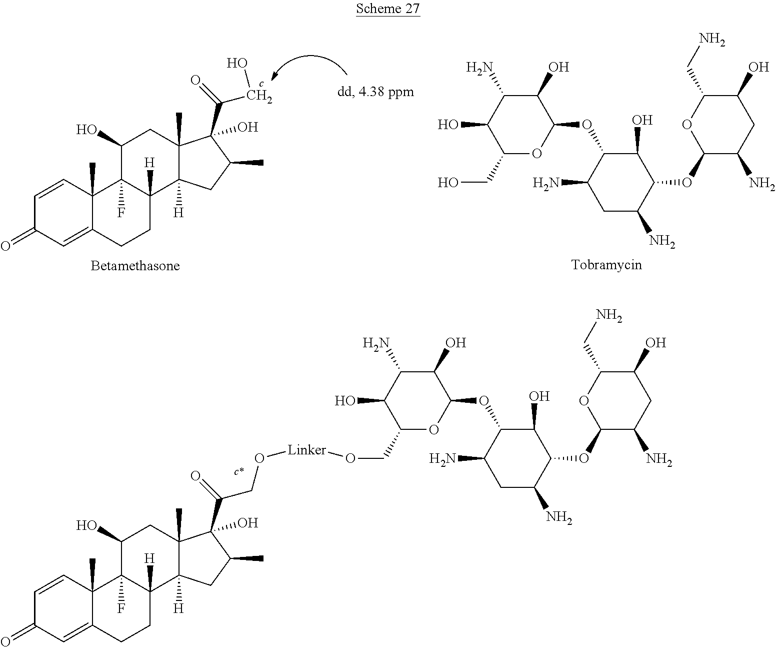

TABLE 26

| *Compound | | δ ppm | |
|---|---|---|---|
| No. Salt | Linker | CH₂^{c}* | MASS |
| HCl 76 | L4 | 5.11 (m) | 946 (MH⁺) |

Tobramycin reacted with dexmethasone to form the following hybrid compounds as shown in Scheme 28 with the results shown in Table 27.

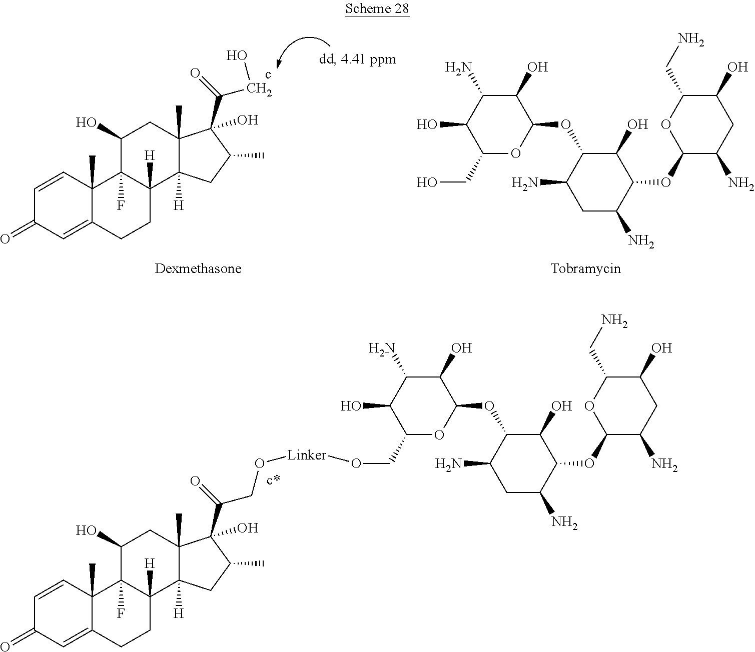

TABLE 27

| *Compound | | δ ppm | |
|---|---|---|---|
| No. Salt | Linker | CH₂^{c}* | MASS |

| | | | | |
|---|---|---|---|---|
| HCl 77 | L14 | 4.98 (dd) | 1028 | (MH⁺) |
| HCl 78 | L4 | 5.07 (dd) | 946 | (MH⁺) |

Tobramycin reacted with prednisolone to form the following hybrid compounds as shown in Scheme 29 with the results shown in Table 28.

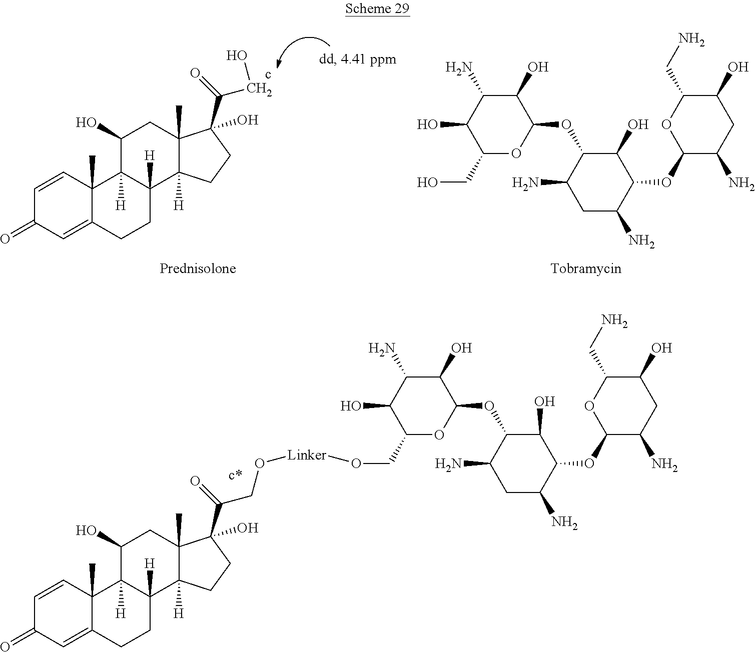

TABLE 28

| *Compound | | δ ppm | | | |
|---|---|---|---|---|---|
| No. Salt | Linker | CH₂^{c}* | | MASS | |
| HCl 79 | L14 | 5.00 | (dd) | 996 | (MH⁺) |
| HCl 80 | L4 | 5.04 | (dd) 300 MHz | 911 | (MH⁺) |
| HCl 81 | L16 | 5.01 | (dd) 300 MHz | | |

| | | | | |
|---|---|---|---|---|
| 84 | L45 | 5.03 | 1186 | (MH⁺) |
| 85 | L46 | 5.03 | 1142 | (MH⁺) |
| 86 | L47 | 5.00 | 1040 | (MH⁺) |
| 105 | L16 | 5.02 | | |

Amikacin reacted with prednisolone to form the following hybrid compounds as shown in Scheme 30 with the results shown in Table 29 and as shown in Scheme 31 with the results shown in Table 30.

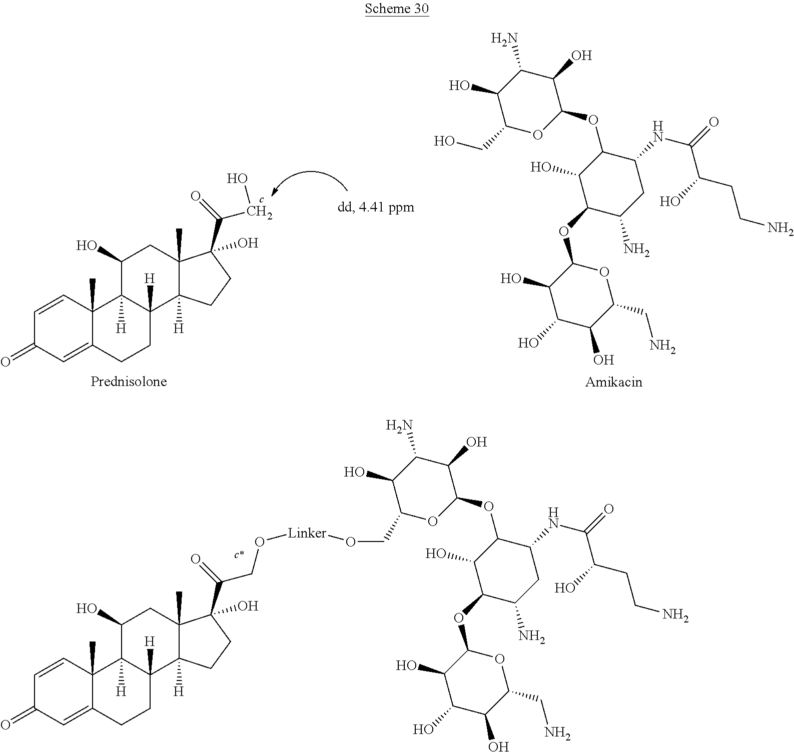

TABLE 29

| *Compound | | δ ppm | |
|---|---|---|---|
| No. Salt | Linker | CH₂^{c}* | MASS |
| 82 HCl | L14 | 5.01 (dd) | 1114 (MH⁺) |

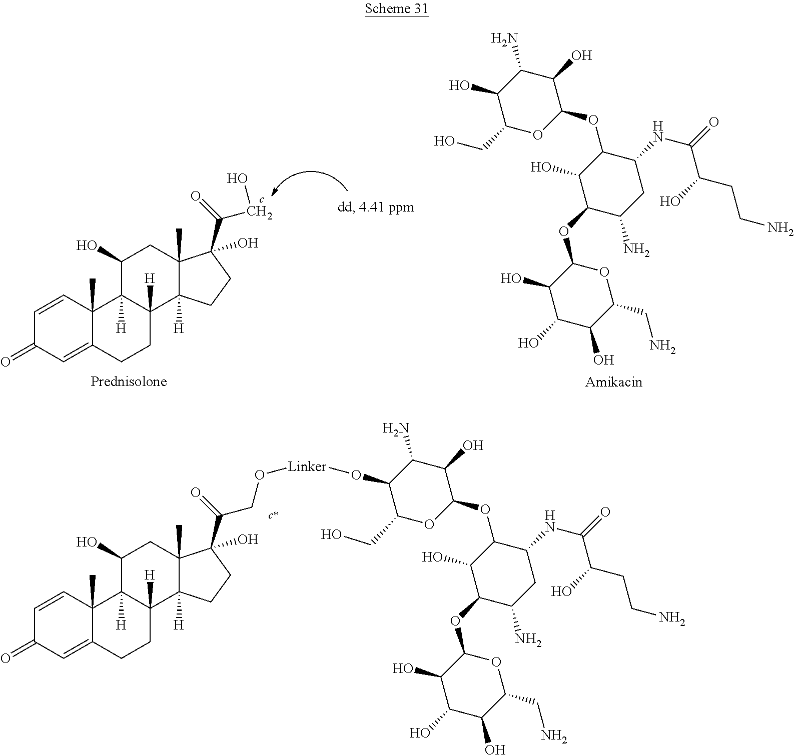

TABLE 30

| *Compound | | δ ppm | |
|---|---|---|---|
| No. Salt | Linker | CH₂^{c}* | MASS |
| 83 HCl | L14 | 5.0 (dd) | 1114 (MH⁺) |

Clindamycin reacted with prednisolone to form the following hybrid compounds as shown in Scheme 32 with the results shown in Table 30.

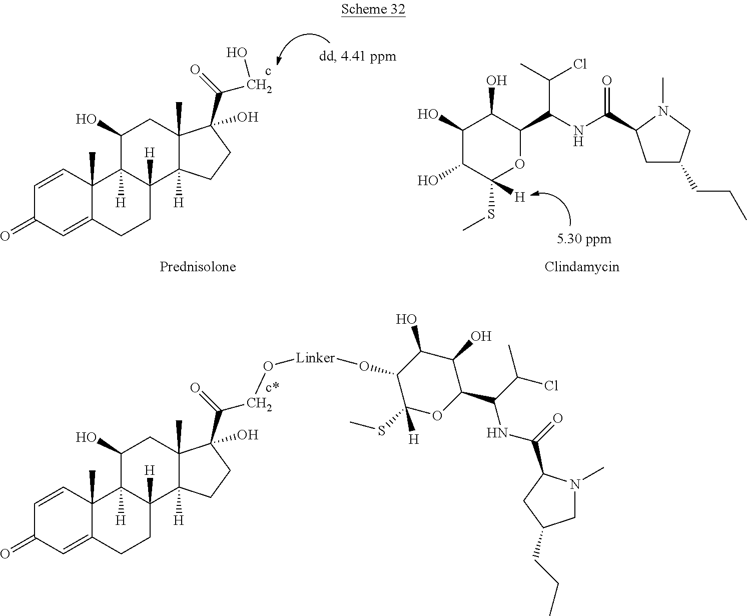

TABLE 30

| *Compound | | δ ppm | |
|---|---|---|---|
| No. | Linker | CH₂^{c}* | MASS |
| 127 | L4 | 5.08 | 867 (MH⁺) |

### BIOLOGICAL EXAMPLES

### Example 1

### In Vitro Metabolic Stability in Rabbit Cornea Homogenates and Human Recombinant Carboxylesterases

Dutch Belted rabbits were euthanized with an overdose of sodium pentobarbital. The corneas were collected and homogenized in ice-cold potassium chloride solution (pH=7.4). The homogenate was centrifuged at 755×g for 30 min at 4° C. and aliquots of the supernatant were stored at or below −70° C. until metabolism experiments were conducted. Prior to storing the homogenates an aliquot was removed for determination of protein concentrations by calculating the 260 nm absorbance using a spectrophotometer. Human recombinant carboxylesterases were purchased from a commercial vendor (BD Gentest™, Bedford, Mass.)

All metabolic stability experiments were performed in triplicate in 96-well plate format. The final incubation mixture contained 1 μM test compound, 0.3 mg/mL corneal protein homogenate or 0.1 mg/mL human recombinant carboxylesterase mixture in a final volume of 0.5 mL 0.1 M potassium phosphate buffer (pH=6.0). The final percentage of solvent in the incubation was less than 1.0% to prevent inhibition of enzymatic activity. Following a pre-incubation at 37° C., test article (i.e. ester linked hybrids) was added to initiate the reaction. At designated time points (typically less than 60 minutes to capture the linear range of metabolite formation), 0.05 mL aliquots were removed from the incubation mixtures using a clean pipet tip and immediately placed in organic solvent to stop any esterase activity.

Hydrolysis of the metabolites was confirmed to be due to esterase activity and not chemical lability. The samples were analyzed by liquid chromatography with mass spectrometry (LC-MS/MS) detection to determine the metabolite concentrations resulting from the metabolism of ester linked hybrids. Internal standards were used to compensate for variability from sample processing, chromatographic elution, mass spectrometer response and ion suppression by matrix components.

### Results

Table 31 lists the rate of metabolite formation in rabbit cornea homogenates

TABLE 31

| | | Rate of | Rate of |
|---|---|---|---|
| | | formation | formation |
| Comp. | | Metabolite 1 | Metabolite 2 |
| No. | IUPAC name | (nM/min/mg) | (nM/min/mg) |
| 73 | (2R,3R)-2-[(dichloroacetyl)amino]-3- | 63.9 ± 7.0 | 16.4 ± 1.5 |
| | hydroxy-3-(4-nitrophenyl)propyl 2- | Betamethasone | Chloramphenicol |
| | [(8S,9R,10S,11S,13S,14S,16S,17R)- | | |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-butanedioate | | |
| 74 | (2R,3R)-2-[(dichloroacetyl)amino]-3- | 51.5 | 19.4 |
| | hydroxy-3-(4-nitrophenyl)propyl 2- | Dexamethasone | Chloramphenicol |
| | [(8S,9R,10S,11S,13S,14S,16R,17R)- | | |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-benzene-1,4- | | |
| | dicarboxylate | | |

Table 32 lists the rate of metabolite formation in human recombinant carboxylesterases

TABLE 32

| | | Rate of | Rate of |
|---|---|---|---|
| | | formation | formation |
| Comp. | | Metabolite 1 | Metabolite 2 |
| No. | IUPAC name | (nM/min/mg) | (nM/min/mg) |
| 73 | (2R,3R)-2-[(dichloroacetyl)amino]-3- | 1440 ± 120 | 107 ± 13 |
| | hydroxy-3-(4-nitrophenyl)propyl 2- | Betamethasone | Chloramphenicol |
| | [(8S,9R,10S,11S,13S,14S,16S,17R)- | | |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-butanedioate | | |
| 18 | 3-({[1-cyclopropyl-6-fluoro-8-methoxy- | 59.7 ± 5.15 | 2.44 ± 0.87 |
| | 7-(3-methylpiperazin-1-yl)-4-oxo-1,4- | Dexamethasone | Gatifloxacin |
| | dihydroquinolin-3- | | |
| | yl]carbonyl}oxy)propyl 2- | | |
| | [(8R,9S,10R,11R,13R,14R,16S,17S)- | | |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-butanedioate | | |
| 54 | 3-[({1-cyclopropyl-6-fluoro-8-methoxy- | 67.8 ± 1.78 | 0.717 ± 0.625 |
| | 7-[(4aR,7aR)-octahydro-6H- | Dexamethasone | Moxifloxacin |
| | pyrrolo[3,4-b]pyridin-6-yl]-4-oxo-1,4- | | |
| | dihydroquinolin-3- | | |
| | yl}carbonyl)oxy]propyl 2- | | |
| | [(8S,9R,10S,11S,13S,14S,16R,17R)- | | |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-benzene-1,4- | | |
| | dicarboxylate | | |
| 2 | rel-1-cyclopropyl-6-fluoro-7-[4-({[(4-{2- | 109 ± 2 | 112 ± 4 |
| | [(9R,10S,11S,13S,16S,17R)-9-fluoro- | Betamethasone | Gatifloxacin |
| | 11,17-dihydroxy-10,13,16-trimethyl-3- | | |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}-4- | | |
| | oxobutanoyl)oxy]methoxy}carbonyl)-3- | | |
| | methylpiperazin-1-yl]-8-methoxy-4- | | |
| | oxo-1,4-dihydroquinoline-3-carboxylic | | |
| | acid | | |
| 64 | [({2-[(8R,10S,11R,13R,14R,17S)- | 641 ± 22 | 644 ± 20 |
| | 11,17-dihydroxy-10,13-dimethyl-3- | Prednisolone | Moxifloxacin |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}carbonyl)oxy]methyl rel-1- | | |
| | cyclopropyl-6-fluoro-8-methoxy-7- | | |
| | [(4aS,7aS)-octahydro-6H-pyrrolo[3,4- | | |
| | b]pyridin-6-yl]-4-oxo-1,4- | | |
| | dihydroquinoline-3-carboxylate | | |
| 65 | 1-[({2-[(8R,10S,11R,13R,14R,17S)- | 364 ± 27 | 482 ± 72 |
| | 11,17-dihydroxy-10,13-dimethyl-3- | Prednisolone | Moxifloxacin |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}carbonyl)oxy]ethyl rel-1- | | |
| | cyclopropyl-6-fluoro-8-methoxy-7- | | |
| | [(4aS,7aS)-octahydro-6H-pyrrolo[3,4- | | |
| | b]pyridin-6-yl]-4-oxo-1,4- | | |
| | dihydroquinoline-3-carboxylate | | |
| 42 | [({2-[(8R,10S,11R,13R,14R,17S)- | 633 ± 85 | 668 ± 102 |
| | 11,17-dihydroxy-10,13-dimethyl-3- | Prednisolone | Gatifloxacin |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}carbonyl)oxy]methyl rel-1- | | |
| | cyclopropyl-6-fluoro-8-methoxy-7-(3- | | |
| | methylpiperazin-1-yl)-4-oxo-1,4- | | |
| | dihydroquinoline-3-carboxylate | | |
| 43 | 1-[({2-[(8R,10S,11R,13R,14R,17S)- | 298 ± 13 | 419 ± 8 |
| | 11,17-dihydroxy-10,13-dimethyl-3- | Prednisolone | Gatifloxacin |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}carbonyl)oxy]ethyl rel-1- | | |
| | cyclopropyl-6-fluoro-8-methoxy-7-(3- | | |
| | methylpiperazin-1-yl)-4-oxo-1,4- | | |
| | dihydroquinoline-3-carboxylate | | |
| 10 | rel-1-cyclopropyl-6-fluoro-7-[4-(4-{2- | 737 ± 28 | 7.71 ± 0.57 |
| | [(8R,9S,10R,11R,13R,14R,16S,17S)- | Dexamethasone | Gatifloxacin |
| | 9-fluoro-11,17-dihydroxy-10,13,16- | | |
| | trimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethoxy}-4-oxobutanoyl)-3- | | |
| | methylpiperazin-1-yl]-8-methoxy-4- | | |
| | oxo-1,4-dihydroquinoline-3-carboxylic | | |
| | acid | | |
| 44 | ({[1-cyclopropyl-6-fluoro-8-methoxy-7- | 365 ± 32 | 760 ± 110 |
| | (3-methylpiperazin-1-yl)-4-oxo-1,4- | Prednisolone | Gatifloxacin |
| | dihydroquinolin-3- | | |
| | yl]carbonyl}oxy)methyl 2- | | |
| | [(8R,10S,11R,13R,14R,17S)-11,17- | | |
| | dihydroxy-10,13-dimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethyl rel-butanedioate | | |
| 25 | rel-1-cyclopropyl-7-[4-({[(4-{2- | 1220 ± 130 | 1400 ± 150 |
| | [(10R,11S,13S,17R)-11,17-dihydroxy- | Prednisolone | Gatifloxacin |
| | 10,13-dimethyl-3-oxo- | | |
| | 6,7,8,9,10,11,12,13,14,15,16,17- | | |
| | dodecahydro-3H- | | |
| | cyclopenta[a]phenanthren-17-yl]-2- | | |
| | oxoethoxy}-4- | | |
| | oxobutanoyl)oxy]methoxy}carbonyl)-3- | | |
| | methylpiperazin-1-yl]-6-fluoro-8- | | |
| | methoxy-4-oxo-1,4-dihydroquinoline- | | |
| | 3-carboxylic acid | | |
| 41 | 4-{2-[(8R,10S,11R,13R,14R,17S)- | 7.60 ± 2.1 | 74.6 ± 4.1 |
| | 11,17-dihydroxy-10,13-dimethyl-3- | Gatifloxacin | Prednisolone |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}-4-oxobutyl rel-1- | | |
| | cyclopropyl-6-fluoro-8-methoxy-7-(3- | | |
| | methylpiperazin-1-yl)-4-oxo-1,4- | | |
| | dihydroquinoline-3-carboxylate | | |
| 59 | rel-1-cyclopropyl-7-[1-({[(4-{2- | 416 ± 192 | 350 ± 180 |
| | [(8R,9R,10S,11R,13R,14R,17S)- | Prednisolone | Moxifloxacin |
| | 11,17-dihydroxy-10,13-dimethyl-3- | | |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}-4- | | |
| | oxobutanoyl)oxy]methoxy}carbonyl)octa- | | |
| | hydro-6H-pyrrolo[3,4-b]pyridin-6-yl]- | | |
| | 6-fluoro-8-methoxy-4-oxo-1,4- | | |
| | dihydroquinoline-3-carboxylic acid | | |
| 29 | rel-1-cyclopropyl-7-{4-[(10R)-14- | 17.3 ± 0.1 | 36.5 ± 1.1 |
| | [(10S,11R,13R,17S)-11,17-dihydroxy- | Prednisolone | Gatifloxacin |
| | 10,13-dimethyl-3-oxo- | | |
| | 6,7,8,9,10,11,12,13,14,15,16,17- | | |
| | dodecahydro-3H- | | |
| | cyclopenta[a]phenanthren-17-yl]- | | |
| | 5,8,11,14-tetraoxo-10-(propan-2-yl)- | | |
| | 2,4,12-trioxa-9-azatetradecan-1-oyl]- | | |
| | 3-methylpiperazin-1-yl}-6-fluoro-8- | | |
| | methoxy-4-oxo-1,4-dihydroquinoline- | | |
| | 3-carboxylic acid | | |
| 61 | rel-1-cyclopropyl-7-{1-[(10R)-14- | 11.8 ± 0.5 | 18.5 ± 1.7 |
| | [(10S,11R,13R,17S)-11,17-dihydroxy- | Prednisolone | Moxifloxacin |
| | 10,13-dimethyl-3-oxo- | | |
| | 6,7,8,9,10,11,12,13,14,15,16,17- | | |
| | dodecahydro-3H- | | |
| | cyclopenta[a]phenanthren-17-yl]- | | |
| | 5,8,11,14-tetraoxo-10-(propan-2-yl)- | | |
| | 2,4,12-trioxa-9-azatetradecan-1- | | |
| | oyl]octahydro-6H-pyrrolo[3,4-b]pyridin- | | |
| | 6-yl}-6-fluoro-8-methoxy-4-oxo-1,4- | | |
| | dihydroquinoline-3-carboxylic acid | | |
| 11 | rel-1-cyclopropyl-6-fluoro-7-[4-({[(4-{2- | 44.5 ± 2.62 | 56 ± 2.1 |
| | [(9R,10S,11S,13S,16R,17R)-9-fluoro- | Dexamethasone | Gatifloxacin |
| | 11,17-dihydroxy-10,13,16-trimethyl-3- | | |
| | oxo-6,7,8,9,10,11,12,13,14,15,16,17- | | |
| | dodecahydro-3H- | | |
| | cyclopenta[a]phenanthren-17-yl]-2- | | |
| | oxoethoxy}-4- | | |
| | oxobutanoyl)oxy]methoxy}carbonyl)-3- | | |
| | methylpiperazin-1-yl]-8-methoxy-4- | | |
| | oxo-1,4-dihydroquinoline-3-carboxylic | | |
| | acid | | |

The data demonstrate linkage of an antibioitic (e.g. chloramphenicol, gatifloxacin, and moxifloxacin) and a steroid (e.g. betamethasone, dexamethasone and prenisolone) as a single hybrid compound was hydrolyzed enzymatically in rabbit cornea homogenates and human recombinant carboxylesterases, to their respective individual antibiotic and steroid drugs. The data suggest that these hybrid compounds will be cleaved in humans to the active metabolites to produce their respective pharmacologic effects.

### Example 2

### In Vitro Corneal Permeability and Metabolic Stability in Human Corneal Epithelial Cells

Clonetics® human corneal epithelial cells (HCEC) were purchased from Lonza Walkersville, Inc. (Walkersville, Md.) pre-seeded on Costar Transwell™ filters in a 24-well plate. Upon receipt HCEC cells were cultured overnight in a 37° C. incubator (95% O₂, 5% CO₂) in media provided by the vendor. Permeability studies were performed within 24 hours of receipt. Dosing solutions 100 μM test article (i.e. ester linked hybrids) were prepared in Lonza's proprietary media by diluting a 50 mM stock solution of the test article in dimethyl sulfoxide. The final percentage of solvent in the incubation was less than 1.0% to prevent inhibition of enzymatic activity or effects on the cell membrane. Transepithelial electrical resistance (TEER) was measured for all wells using a voltohmmeter with STX-2 electrodes (World Precision Instruments Inc., Sarasota, Fla.) after adding 100 μL pre-warmed (37° C.) media to the apical compartment. All permeability experiments were performed in triplicate by adding 100 μL of the 100 μM dosing solution to the apical compartment of each well (final incubation concentration of 50 μM). After a 2 hour incubation, aliquots of medium from the basolateral compartment of each well were removed to assess permeability. Aliquots of the dosing solution from the apical compartment of each well were collected at the end of incubation to assess mass balance. A final TEER value was measured and recorded for all wells.

To evaluate human corneal epithelial cell integrity incubations were conducted using 2 μCi/mL ³H-mannitol for the same 2 hour incubation period with aliquots taken from the basolateral compartment. ³H-Mannitol samples were analyzed using liquid scintillation counting. All ester linked hybrids samples were analyzed by liquid chromatography with mass spectrometry (LC-MS/MS) detection to determine the parent (i.e., ester linked hybrids) and metabolite (i.e. steroid and antibiotic) concentrations resulting from the metabolism of ester linked hybrids. Internal standards were used to compensate for variability from sample processing, chromatographic elution, mass spectrometer response and ion suppression by matrix components.

### Results

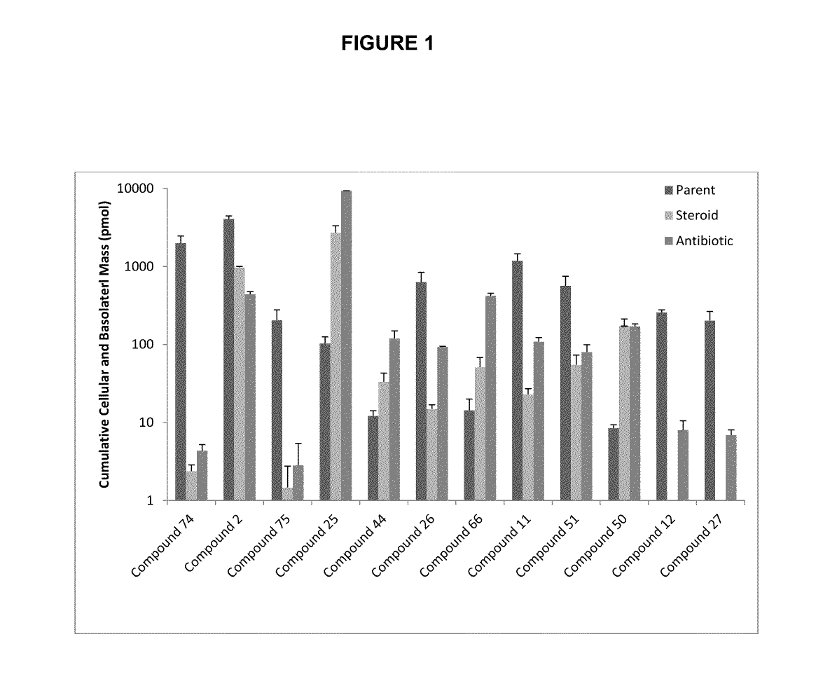

FIG. 1 shows the cellular uptake of ester linked hybrid (parent) compounds and the hydrolyzed metabolites (steroid and antibiotic) after a two hour incubation with Human Corneal Epithelial Cells. The data demonstrate that linkage of an antibioitic (e.g. chloramphenicol, gatifloxacin, and moxifloxacin) and a steroid (e.g. betamethasone, dexamethasone and prenisolone) as a single hybrid compound was taken up into human corneal epithelial cells and enzymatically hydrolyzed to the individual antibiotic and steroid.

### Example 3

### Ocular Pharmacokinetics of Compounds 11, 25, 44, and 66 Following a Single Topical Ophthalmic Administration in New Zealand White Rabbits

Rabbits were dosed once by ocular instillation to both eyes with each compound formulated in a 0.4% (w/v) solution. At 0.25, 0.5, 1, 2, 6, and 10 hours post dose cornea, aqueous humor, conjunctiva and eyelid margin were collected and stored at approximately −70° C. until bioanalysis. Ocular tissue samples were analyzed by liquid chromatography with mass spectrometry (LC-MS/MS) detection to determine the parent (i.e., ester linked hybrids) and metabolite (i.e. steroid and antibiotic) concentrations resulting from the metabolism of ester linked hybrids. Internal standards were used to compensate for variability from sample processing, chromatographic elution, mass spectrometer response and ion suppression by matrix components.

### Results

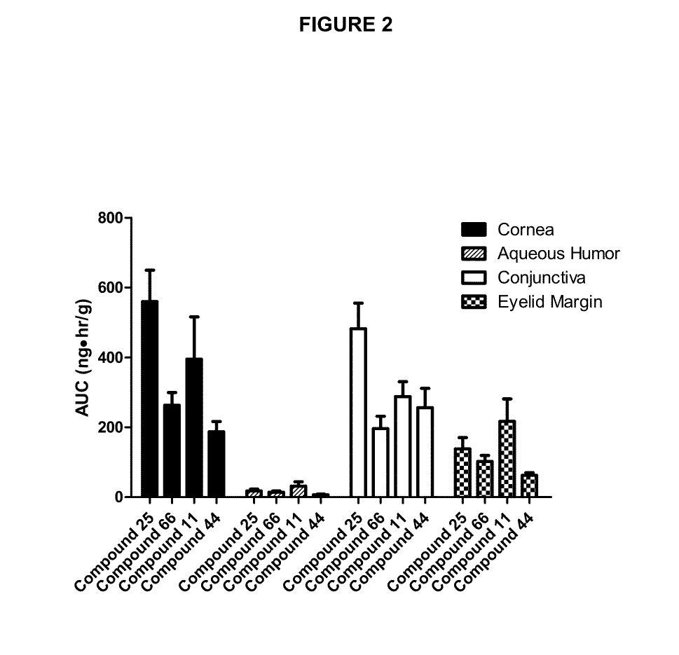

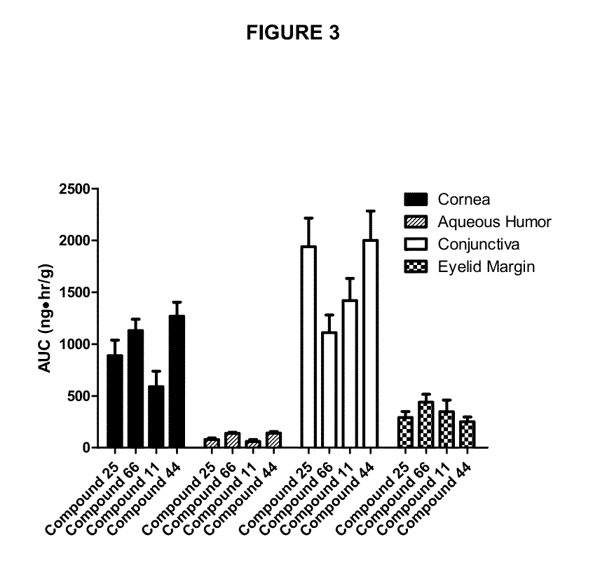

FIG. 2 shows the mean±standard error of the enzymatically cleaved steroid area under the concentration-time profile (AUC₀₋₁₀ₕᵣ) Following a Single Topical Ocular Dose of 0.4% of the Hybrid Compound in Rabbits.
FIG. 3 shows the mean±standard error of the enzymatically cleaved antibiotic area under the concentration-time profile (AUC₀₋₁₀ₕᵣ) Following a Single Topical Ocular Dose 10 hr, of 0.4% of the Hybrid Compound in Rabbits.

The data demonstrate that linkage of an antibioitic (e.g. gatifloxacin and moxifloxacin) and a steroid (e.g. dexamethasone and prednisolone) as a single hybrid compound was taken up into rabbit ocular tissues and enzymatically hydrolyzed to the individual antibiotic and steroid. This animal study shows that these hybrid compounds have the capability to penetrate ocular tissues and get cleaved to the active metabolites to be clinically effective in treating inflammatory and infectious diseases.

What is claimed is:

1. A hybrid drug comprising at one antibiotic moieties and one steroid moiety, or a pharmaceutical salt thereof, which are connected via two separate covalent bonds to a linker such that said covalent bonds degrade in vivo to yield the respective antibiotics independently.

2. The hybrid drug according to claim 1 wherein the antibiotic moiety is selected from the group consisting of levofloxacin, moxifloxacin, gatifloxacin, clindamycin, besifloxacin, gemifloxacin, trovafloxacin, ofloxacin, ciprofloxacin, sparfloxacin, grepafloxacin, norfoxacin, enoxacin, lomefloxacin, fleroxacin, tosufloxacin, prulifloxacin, pazufloxacin, clinafloxacin, garenoxacin, sitafloxacin, loracarbef, cephalexin, cefuroxime, ceftriaxone, ceftaxime, ceftizoxime, ceftibuten, ceftazidime, cefprozil, cefpodoxime, cefoxitin, cefotetan, cefotaxime, cefoperazone, cefixime, cefepime, cefditoren, cefdinir, cefoperaxone, moxalactam, cefazolin, cefamandole, cefadroxil, cefaclor, cephalothin, cephradine, cephacetrile, cephalothin, chloramphenicol, tobramycin, streptomycin, gentamicin, kanamycin, amikacin, netilmicin, penicillin g, ticarcillin, methicillin, phenthicillin, cloxacillin, dicloxacillin, nafcillin, oxacillin, erythromycin and azithromycin.

3. The hybrid drug according to claim 1 wherein the steroid moiety is selected from:
dexmethasone, betamethasone, triamcinolone acetonide, prednisolone and hydrocortisone.

4. The hybrid drug according to claim 1 wherein the antibiotic moiety is selected from:
moxifloxacin, besifloxacin, gatifloxacin, amikacin chloramphenicol, tobramycin and clindamycin.

5. The hybrid drug according to claim 1 wherein the antibiotic moiety is:
gatifloxacin, and the steroid moiety is selected from: dexmethasone, betamethasone, triamcinolone acetonide, prednisolone and hydrocortisone.

6. The hybrid drug according to claim 1 wherein the antibiotic moiety is:
moxifloxacin, and the steroid moiety is selected from: dexmethasone, betamethasone, triamcinolone acetonide, prednisolone and hydrocortisone.

7. The hybrid compound according to claim 1, wherein said linker comprises an ester, a carboxylate, a carbonyl, a carbonate, an amido, a carbamate, a ketone, an amino, an oxo, an ethylene glycol, a polyethylene glycol or an alkylene moiety.

8. The hybrid compound according to claim 1, comprising a linker having two bonds, wherein said bonds are asymmetrically degraded in vivo to release independently the antibiotic moiety and the steroid moiety.

9. The compound according to claim 1, selected from:
rel-1-cyclopropyl-7-[4-(5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl)-3-methylpiperazin-1-yl]-6-fluoro-8-methoxy-4-oxo-1,4-dihydroquinoline-3-carboxylic acid;
rel-7-[4-({[(4-amino-5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
rel-7-{4-[(4R)-4-amino-5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl]-3-methylpiperazin-1-yl}-1-cyclopropyl-6-fluoro-8-methoxy-4-oxo-1,4-dihydroquinoline-3-carboxylic acid;
rel-7-{4-[(2R)-2-amino-4-{2-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl]-3-methylpiperazin-1-yl}-1-cyclopropyl-6-fluoro-8-methoxy-4-oxo-1,4-dihydroquinoline-3-carboxylic acid;
rel-7-{4-[({[(3R)-3-amino-5-{2-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl]oxy}methoxy)carbonyl]-3-methylpiperazin-1-yl}-1-cyclopropyl-6-fluoro-8-methoxy-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-pentanedioate;
rel-7-(4-{7-carboxy-15-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,9,12,15-tetraoxo-2,4,13-trioxa-8-azapentadecan-1-oyl}-3-methylpiperazin-1-yl)-1-cyclopropyl-6-fluoro-8-methoxy-4-oxo-1,4-dihydroquinoline-3-carboxylic acid;
rel-7-(4-{2-amino-3-[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]propanoyl}-3-methylpiperazin-1-yl)-1-cyclopropyl-6-fluoro-8-methoxy-4-oxo-1,4-dihydroquinoline-3-carboxylic acid;
rel-1-cyclopropyl-7-[4-({[(5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl)oxy]methoxy}carbonyl)-3-methylpiperazin-1-yl]-6-fluoro-8-methoxy-4-oxo-1,4-dihydroquinoline-3-carboxylic acid;
rel-7-[4-({[(3-amino-4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
rel-7-(4-{6-amino-15-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-5,9,12,15-tetraoxo-2,4,8,13-tetraoxapentadecan-1-oyl}-3-methylpiperazin-1-yl)-1-cyclopropyl-6-fluoro-8-methoxy-4-oxo-1,4-dihydroquinoline-3-carboxylic acid;
rel-7-(4-{7-carboxy-15-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-5,9,12,15-tetraoxo-2,4,13-trioxa-8-azapentadecan-1-oyl}-3-methylpiperazin-1-yl)-1-cyclopropyl-6-fluoro-8-methoxy-4-oxo-1,4-dihydroquinoline-3-carboxylic acid;
rel-1-cyclopropyl-6-fluoro-7-{1-[(10R)-14-[(9S,10R,11R,13R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,8,11,14-tetraoxo-10-(propan-2-yl)-2,4,12-trioxa-9-azatetradecan-1-oyl]octahydro-6H-pyrrolo[3,4-b]pyridin-6-yl}-8-methoxy-4-oxo-1,4-dihydroquinoline-3-carboxylic acid;
rel-1-cyclopropyl-6-fluoro-7-{4-[(10R)-14-[(9S,10R,11R,13R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,8,11,14-tetraoxo-10-(propan-2-yl)-2,4,12-trioxa-9-azatetradecan-1-oyl]-3-methylpiperazin-1-yl}-8-methoxy-4-oxo-1,4-dihydroquinoline-3-carboxylic acid;
rel-1-cyclopropyl-7-{1-[(10R)-14-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-5,8,11,14-tetraoxo-10-(propan-2-yl)-2,4,12-trioxa-9-azatetradecan-1-oyl]octahydro-6H-pyrrolo[3,4-b]pyridin-6-yl}-6-fluoro-8-methoxy-4-oxo-1,4-dihydroquinoline-3-carboxylic acid;
rel-1-cyclopropyl-7-{4-[(10R)-14-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-5,8,11,14-tetraoxo-10-(propan-2-yl)-2,4,12-trioxa-9-azatetradecan-1-oyl]-3-methylpiperazin-1-yl}-6-fluoro-8-methoxy-4-oxo-1,4-dihydroquinoline-3-carboxylic acid;
rel-7-[4-({[(2-amino-4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)-3-methylpiperazin-1-yl]-1-cyclopropyl-6-fluoro-8-methoxy-4-oxo-1,4-dihydroquinoline-3-carboxylic acid;
[({[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methoxy}carbonyl)oxy]methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
[({7-[(3R)-3-aminoazepan-1-yl]-8-chloro-1-cyclopropyl-6-fluoro-4-oxo-1,4-dihydroquinolin-3-yl}carbonyl)oxy]methyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
[({7-[(3R)-3-aminoazepan-1-yl]-8-chloro-1-cyclopropyl-6-fluoro-4-oxo-1,4-dihydroquinolin-3-yl}carbonyl)oxy]methyl 2-[(10R,11S,13S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethyl rel-butanedioate;
({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
({[1-cyclopropyl-6-fluoro-8-methoxy-7-(3-methylpiperazin-1-yl)-4-oxo-1,4-dihydroquinolin-3-yl]carbonyl}oxy)methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methyl 2-[(10R,11S,13S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethyl rel-butanedioate;
({[1-cyclopropyl-6-fluoro-8-methoxy-7-(3-methylpiperazin-1-yl)-4-oxo-1,4-dihydroquinolin-3-yl]carbonyl}oxy)methyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
({[1-cyclopropyl-6-fluoro-8-methoxy-7-(3-methylpiperazin-1-yl)-4-oxo-1,4-dihydroquinolin-3-yl]carbonyl}oxy)methyl 2-[(10R,11S,13S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethyl rel-butanedioate;
rel-1-cyclopropyl-6-fluoro-7-[1-({[(4-{2-[(9R,10S,11S,13S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)octahydro-6H-pyrrolo[3,4-b]pyridin-6-yl]-8-methoxy-4-oxo-1,4-dihydroquinoline-3-carboxylic acid;
rel-1-cyclopropyl-7-[1-({[(4-{2-[(10R,11S,13S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethoxy}-4-oxobutanoyl)oxy]methoxy}carbonyl)octahydro-6H-pyrrolo[3,4-b]pyridin-6-yl]-6-fluoro-8-methoxy-4-oxo-1,4-dihydroquinoline-3-carboxylic acid;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl 2-[(9R,10S,11S,13S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
rel-1-cyclopropyl-7-(1-{[*{[4-({2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)phenyl]carbonyl}oxy)methoxy]carbonyl}octahydro-6H-pyrrolo[3,4-b]pyridin-6-yl)-6-fluoro-8-methoxy-4-oxo-1,4-dihydroquinoline-3-carboxylic acid;
rel-1-cyclopropyl-7-(4-{[({[4-({2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)phenyl]carbonyl}oxy)methoxy]carbonyl}-3-methylpiperazin-1-yl)-6-fluoro-8-methoxy-4-oxo-1,4-dihydroquinoline-3-carboxylic acid;
rel-1-cyclopropyl-6-fluoro-7-(4-{[({[4-({2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)phenyl]carbonyl}oxy)methoxy]carbonyl}octahydro-6H-pyrrolo[3,4-b]pyridin-6-yl)-8-methoxy-4-oxo-1,4-dihydroquinoline-3-carboxylic acid;
rel-1-cyclopropyl-6-fluoro-7-(4-{[({[4-({2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)phenyl]carbonyl}oxy)methoxy]carbonyl}-3-methylpiperazin-1-yl)-8-methoxy-4-oxo-1,4-dihydroquinoline-3-carboxylic acid;
({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methyl 2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
({[1-cyclopropyl-6-fluoro-8-methoxy-7-(3-methylpiperazin-1-yl)-4-oxo-1,4-dihydroquinolin-3-yl]carbonyl}oxy)methyl 2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
rel-1-cyclopropyl-7-[4-({[(4-{2-[(10R,11S,13S,17R)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethoxy}-4-oxobutanoyl)oxy]methoxy}carbonyl)-3-methylpiperazin-1-yl]-6-fluoro-8-methoxy-4-oxo-1,4-dihydroquinoline-3-carboxylic acid;
rel-1-cyclopropyl-6-fluoro-7-[1-({[(4-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)octahydro-6H-pyrrolo[3,4-b]pyridin-6-yl]-8-methoxy-4-oxo-1,4-dihydroquinoline-3-carboxylic acid;
rel-1-cyclopropyl-6-fluoro-7-[4-({[(4-{2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)-3-methylpiperazin-1-yl]-8-methoxy-4-oxo-1,4-dihydroquinoline-3-carboxylic acid;
rel-1-cyclopropyl-7-[4-({1-[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]ethoxy}carbonyl)-3-methylpiperazin-1-yl]-6-fluoro-8-methoxy-4-oxo-1,4-dihydroquinoline-3-carboxylic acid;
({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methyl 2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
rel-1-cyclopropyl-7-[1-({[(4-{2-[(8R,9R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)octahydro-6H-pyrrolo[3,4-b]pyridin-6-yl]-6-fluoro-8-methoxy-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
rel-1-cyclopropyl-7-[4-({[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)-3-methylpiperazin-1-yl]-6-fluoro-8-methoxy-4-oxo-1,4-dihydroquinoline-3-carboxylic acid;
1-[({2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)oxy]ethyl rel-1-cyclopropyl-6-fluoro-8-methoxy-7-(3-methylpiperazin-1-yl)-4-oxo-1,4-dihydroquinoline-3-carboxylate;
[({2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)oxy]methyl rel-1-cyclopropyl-6-fluoro-8-methoxy-7-(3-methylpiperazin-1-yl)-4-oxo-1,4-dihydroquinoline-3-carboxylate;
1-[({2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)oxy]ethyl rel-1-cyclopropyl-6-fluoro-8-methoxy-7-[(4aS,7aS)-octahydro-6H-pyrrolo[3,4-b]pyridin-6-yl]-4-oxo-1,4-dihydroquinoline-3-carboxylate;
[({2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)oxy]methyl rel-1-cyclopropyl-6-fluoro-8-methoxy-7-[(4aS,7aS)-octahydro-6H-pyrrolo[3,4-b]pyridin-6-yl]-4-oxo-1,4-dihydroquinoline-3-carboxylate;
(2R,3R)-2-[(dichloroacetyl)amino]-3-hydroxy-3-(4-nitrophenyl)propyl 2-[(8S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
rel-1-cyclopropyl-6-fluoro-7-[4-({[(4-{2-[(9R,10S,11S,13S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)-3-methylpiperazin-1-yl]-8-methoxy-4-oxo-1,4-dihydroquinoline-3-carboxylic acid;
4-{[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methoxy}-4-oxobutyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
4-{[(2R,3S,4R,5R,6S)-4-amino-6-{[(1S,2S,3R,4S,6R)-4-amino-6-{[(2S)-4-amino-2-hydroxybutanoyl]amino}-3-{[(2R,3R,4S,5S,6R)-6-(aminomethyl)-3,4,5-trihydroxytetrahydro-2H-pyran-2-yl]oxy}-2-hydroxycyclohexyl]oxy}-5-hydroxy-2-(hydroxymethyl)tetrahydro-2H-pyran-3-yl]oxy}-4-oxobutyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
4-{[(2R,3S,4S,5R,6S)-4-amino-6-{[(1S,2S,3R,4S,6R)-4-amino-6-{[(2S)-4-amino-2-hydroxybutanoyl]amino}-3-{[(2R,3R,4S,5S,6R)-6-(aminomethyl)-3,4,5-trihydroxytetrahydro-2H-pyran-2-yl]oxy}-2-hydroxycyclohexyl]oxy}-3,5-dihydroxytetrahydro-2H-pyran-2-yl]methoxy}-4-oxobutyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
2-{2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}ethyl rel-1-cyclopropyl-6-fluoro-8-methoxy-7-(3-methylpiperazin-1-yl)-4-oxo-1,4-dihydroquinoline-3-carboxylate;
4-{[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methoxy}-4-oxobutyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
4-{2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-(3-methylpiperazin-1-yl)-4-oxo-1,4-dihydroquinoline-3-carboxylate;
4-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-[(4aS,7aS)-octahydro-6H-pyrrolo[3,4-b]pyridin-6-yl]-4-oxo-1,4-dihydroquinoline-3-carboxylate;
4-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-(3-methylpiperazin-1-yl)-4-oxo-1,4-dihydroquinoline-3-carboxylate;
4-{2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-[(4aS,7aS)-octahydro-6H-pyrrolo[3,4-b]pyridin-6-yl]-4-oxo-1,4-dihydroquinoline-3-carboxylate;
4-{2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-(3-methylpiperazin-1-yl)-4-oxo-1,4-dihydroquinoline-3-carboxylate;
2-[(8R,9R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-4-[({1-cyclopropyl-7-[(4aS,7aS)-4a,7a-dimethyloctahydro-6H-pyrrolo[3,4-b]pyridin-6-yl]-6-fluoro-8-methoxy-4-oxo-1,4-dihydroquinolin-3-yl}carbonyl)amino]butanoate;
2-{2-[2-({[1-cyclopropyl-6-fluoro-8-methoxy-7-(3-methylpiperazin-1-yl)-4-oxo-1,4-dihydroquinolin-3-yl]carbonyl}oxy)ethoxy]ethoxy}ethyl 2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
3-[({1-cyclopropyl-6-fluoro-8-methoxy-7-[(4aR,7aR)-octahydro-6H-pyrrolo[3,4-b]pyridin-6-yl]-4-oxo-1,4-dihydroquinolin-3-yl}carbonyl)oxy]propyl 2-[(8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
3-[({1-cyclopropyl-6-fluoro-8-methoxy-7-[(4aR,7aR)-octahydro-6H-pyrrolo[3,4-b]pyridin-6-yl]-4-oxo-1,4-dihydroquinolin-3-yl}carbonyl)oxy]propyl 2-[(8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-(2E)-but-2-enedioate;
3-[({1-cyclopropyl-6-fluoro-8-methoxy-7-[(4aR,7aR)-octahydro-6H-pyrrolo[3,4-b]pyridin-6-yl]-4-oxo-1,4-dihydroquinolin-3-yl}carbonyl)oxy]propyl 2-[(8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
4-({2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)benzyl rel-1-cyclopropyl-6-fluoro-8-methoxy-7-(3-methylpiperazin-1-yl)-4-oxo-1,4-dihydroquinoline-3-carboxylate;
(2R,3R)-2-[(dichloroacetyl)amino]-3-hydroxy-3-(4-nitrophenyl)propyl 2-[(8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
2-({[1-cyclopropyl-6-fluoro-8-methoxy-7-(3-methylpiperazin-1-yl)-4-oxo-1,4-dihydroquinolin-3-yl]carbonyl}oxy)ethyl 2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-(2E)-but-2-enedioate;
2-({[1-cyclopropyl-6-fluoro-8-methoxy-7-(3-methylpiperazin-1-yl)-4-oxo-1,4-dihydroquinolin-3-yl]carbonyl}oxy)ethyl 2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
4-({2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)benzyl rel-1-cyclopropyl-6-fluoro-8-methoxy-7-(3-methylpiperazin-1-yl)-4-oxo-1,4-dihydroquinoline-3-carboxylate;
2-{2-[2-({[1-cyclopropyl-6-fluoro-8-methoxy-7-(3-methylpiperazin-1-yl)-4-oxo-1,4-dihydroquinolin-3-yl]carbonyl}oxy)ethoxy]ethoxy}ethyl 2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
2-[(8R,9R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-4-({[1-cyclopropyl-6-fluoro-8-methoxy-7-(3-methylpiperazin-1-yl)-4-oxo-1,4-dihydroquinolin-3-yl]carbonyl}amino)butanoate;
3-({[1-cyclopropyl-6-fluoro-8-methoxy-7-(3-methylpiperazin-1-yl)-4-oxo-1,4-dihydroquinolin-3-yl]carbonyl}oxy)propyl 2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
(2R,3R)-2-[(dichloroacetyl)amino]-3-hydroxy-3-(4-nitrophenyl)propyl 2-[(8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
3-({[1-cyclopropyl-6-fluoro-8-methoxy-7-(3-methylpiperazin-1-yl)-4-oxo-1,4-dihydroquinolin-3-yl]carbonyl}oxy)propyl 2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate;
2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-3-({[1-cyclopropyl-6-fluoro-8-methoxy-7-(3-methylpiperazin-1-yl)-4-oxo-1,4-dihydroquinolin-3-yl]carbonyl}amino)propanoate;
rel-1-cyclopropyl-6-fluoro-7-[4-(4-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)-3-methylpiperazin-1-yl]-8-methoxy-4-oxo-1,4-dihydroquinoline-3-carboxylic acid;
rel-1-cyclopropyl-6-fluoro-7-[4-(2-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-2-oxoethyl)-3-methylpiperazin-1-yl]-8-methoxy-4-oxo-1,4-dihydroquinoline-3-carboxylic acid;
2-{2-[(8R,9S,10R,11R,13R,14R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-2-oxoethyl rel-1-cyclopropyl-6-fluoro-8-methoxy-7-(3-methylpiperazin-1-yl)-4-oxo-1,4-dihydroquinoline-3-carboxylate;
rel-1-cyclopropyl-6-fluoro-7-[4-(4-{2-[(8R,9S,10R,11R,13R,14R,16R,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutanoyl)-3-methylpiperazin-1-yl]-8-methoxy-4-oxo-1,4-dihydroquinoline-3-carboxylic acid;
[({7-[(3R)-3-aminoazepan-1-yl]-8-chloro-1-cyclopropyl-6-fluoro-4-oxo-1,4-dihydroquinolin-3-yl}carbonyl)oxy]methyl 2-[(8S,10R,11S,13S,14S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-4,18-dioxo-5,8,11,14,17-pentaoxahenicosane-1,21-dioate;
[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-4,15-dioxo-5,8,11,14-tetraoxaoctadecane-1,18-dioate;
1-{[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methyl}4′-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl}1′,4-methanediyl reldi-butanedioate;
rel-1-cyclopropyl-6-fluoro-7-(1-{12-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,9,12-trioxo-2,4,7,10-tetraoxadodecan-1-oyl}octahydro-6H-pyrrolo[3,4-b]pyridin-6-yl)-8-methoxy-4-oxo-1,4-dihydroquinoline-3-carboxylic acid;
rel-1-cyclopropyl-6-fluoro-7-(4-{12-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,9,12-trioxo-2,4,7,10-tetraoxadodecan-1-oyl}-3-methylpiperazin-1-yl)-8-methoxy-4-oxo-1,4-dihydroquinoline-3-carboxylic acid;
rel-7-(1-{7-carboxy-15-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-5,9,12,15-tetraoxo-2,4,13-trioxa-8-azapentadecan-1-oyl}octahydro-6H-pyrrolo[3,4-b]pyridin-6-yl)-1-cyclopropyl-6-fluoro-8-methoxy-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 2-[(9R,10S,11S,13S,16R,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-benzene-1,4-dicarboxylate;
rel-1-cyclopropyl-7-(1-{12-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-5,9,12-trioxo-2,4,7,10-tetraoxadodecan-1-oyl}octahydro-6H-pyrrolo[3,4-b]pyridin-6-yl)-6-fluoro-8-methoxy-4-oxo-1,4-dihydroquinoline-3-carboxylic acid;
rel-1-cyclopropyl-7-(4-{12-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-5,9,12-trioxo-2,4,7,10-tetraoxadodecan-1-oyl}-3-methylpiperazin-1-yl)-6-fluoro-8-methoxy-4-oxo-1,4-dihydroquinoline-3-carboxylic acid;
1-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl}4-{[({2-[(9R,10S,11S,13S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)oxy]methyl}rel-(2R)-2-aminobutanedioate;
({[1-cyclopropyl-6-fluoro-8-methoxy-7-(3-methylpiperazin-1-yl)-4-oxo-1,4-dihydroquinolin-3-yl]carbonyl}oxy)methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-3-hydroxypentanedioate;
rel-1-cyclopropyl-7-[1-({[(5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-3-hydroxy-5-oxopentanoyl)oxy]methoxy}carbonyl)octa hydro-6H-pyrrolo[3,4-b]pyridin-6-yl]-6-fluoro-8-methoxy-4-oxo-1,4-dihydroquinoline-3-carboxylic acid;
rel-1-cyclopropyl-7-[4-({[(5-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-3-hydroxy-5-oxopentanoyl)oxy]methoxy}carbonyl)-3-methylpiperazin-1-yl]-6-fluoro-8-methoxy-4-oxo-1,4-dihydroquinoline-3-carboxylic acid;
rel-7-{4-[(2R)-2-amino-5-{2-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-5-oxopentanoyl]-3-methylpiperazin-1-yl}-1-cyclopropyl-6-fluoro-8-methoxy-4-oxo-1,4-dihydroquinoline-3-carboxylic acid;
({[1-cyclopropyl-6-fluoro-8-methoxy-7-(3-methylpiperazin-1-yl)-4-oxo-1,4-dihydroquinolin-3-yl]carbonyl}oxy)methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-3-aminopentanedioate;
rel-1-cyclopropyl-7-{4-[(7R)-7-(2-{2-[(10S,11R,13R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-2-oxoethyl)-11,11-dimethyl-5,9-dioxo-2,4,10-trioxa-8-azadodecan-1-oyl]-3-methylpiperazin-1-yl}-6-fluoro-8-methoxy-4-oxo-1,4-dihydroquinoline-3-carboxylic acid;
rel-7-(4-{2-[(tert-butoxycarbonyl)amino]-3-[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]propanoyl}-3-methylpiperazin-1-yl)-1-cyclopropyl-6-fluoro-8-methoxy-4-oxo-1,4-dihydroquinoline-3-carboxylic acid;
rel-1-cyclopropyl-7-{4-[7-({2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}carbonyl)-11,11-dimethyl-5,9-dioxo-2,4,10-trioxa-8-azadodecan-1-oyl]-3-methylpiperazin-1-yl}-6-fluoro-8-methoxy-4-oxo-1,4-dihydroquinoline-3-carboxylic acid;
rel-1-cyclopropyl-7-[4-(6-{[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methyl}-10,10-dimethyl-5,8-dioxo-2,4,9-trioxa-7-azaundecan-1-oyl)-3-methylpiperazin-1-yl]-6-fluoro-8-methoxy-4-oxo-1,4-dihydroquinoline-3-carboxylic acid;
rel-1-cyclopropyl-6-fluoro-7-{1-[(10R)-14-[(9S,10R,11R,13R,16S,17S)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-5,8,11,14-tetraoxo-10-(propan-2-yl)-2,4,12-trioxa-9-azatetradecan-1-oyl]octahydro-6H-pyrrolo[3,4-b]pyridin-6-yl}-8-methoxy-4-oxo-1,4-dihydroquinoline-3-carboxylic acid;
rel-1-cyclopropyl-7-{4-[6-(2-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-2-oxoethyl)-10,10-dimethyl-5,8-dioxo-2,4,9-trioxa-7-azaundecan-1-oyl]-3-methylpiperazin-1-yl}-6-fluoro-8-methoxy-4-oxo-1,4-dihydroquinoline-3-carboxylic acid;
[({[(2R,3S,4S,5R,6S)-4-amino-6-{[(1S,2S,3R,4S,6R)-4,6-diamino-3-{[(2R,3R,5S,6R)-3-amino-6-(aminomethyl)-5-hydroxytetrahydro-2H-pyran-2-yl]oxy}-2-hydroxycyclohexyl]oxy}-3,5-dihydroxytetrahydro-2H-pyran-2-yl]methoxy}carbonyl)oxy]methyl 2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
{[(1-cyclopropyl-6-fluoro-8-methoxy-7-{1-[(5-methyl-2-oxo-1,3-dioxol-4-yl)methyl]octahydro-6H-pyrrolo[3,4-b]pyridin-6-yl}-4-oxo-1,4-dihydroquinolin-3-yl)carbonyl]oxy}methyl 2-[(8R,10S,11R,13R,14R,17S)-11,17-dihydroxy-10,13-dimethyl-3-oxo-2,3,6,7,8,9,10,11,12,13,14,15,16,17-tetradecahydro-1H-cyclopenta[a]phenanthren-17-yl]-2-oxoethyl rel-butanedioate;
{[(1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinolin-3-yl)carbonyl]oxy}methyl 2-[(8R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
2-[2-(2-{[(1-cyclopropyl-6-fluoro-8-methoxy-7-{3-methyl-4-[(5-methyl-2-oxo-1,3-dioxol-4-yl)methyl]piperazin-1-yl}-4-oxo-1,4-dihydroquinol in-3-yl)carbonyl]oxy}ethoxy)ethoxy]ethyl 2-[(8R,9R,10S,11R,13R,14R,17S)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethyl rel-butanedioate;
1-cyclopropyl-7-(4-{[({[1-cyclopropyl-6-fluoro-8-methoxy-7-(octahydro-6H-pyrrolo[3,4-b]pyridin-6-yl)-4-oxo-1,4-dihydroquinolin-3-yl]carbonyl}oxy)methoxy]carbonyl}-3-methylpiperazin-1-yl)-6-fluoro-8-methoxy-4-oxo-1,4-dihydroquinoline-3-carboxylic acid;
8-chloro-1-cyclopropyl-7-{3-[({[(4-{2-[(10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutanoyl)oxy]methoxy}carbonyl)amino]azepan-1-yl}-6-fluoro-4-oxo-1,4-dihydroquinoline-3-carboxylic acid;
4-{2-[(8R,9S,10R,11R,13R,14R,16R,17S)-17-(butanoyloxy)-9-fluoro-11-hydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-(3-methylpiperazin-1-yl)-4-oxo-1,4-dihydroquinoline-3-carboxylate;
4-{[(8R,9S,10R,11R,13R,14R,16R,17S)-17-{[(2,2-dimethylpropanoyl)oxy]acetyl}-9-fluoro-11-hydroxy-10,13,16-trimethyl-3-oxo-6,7,8,9,10,11,12,13,14,15,16,17-dodecahydro-3H-cyclopenta[a]phenanthren-17-yl]oxy}-4-oxobutyl rel-1-cyclopropyl-6-fluoro-8-methoxy-7-(3-methylpiperazin-1-yl)-4-oxo-1,4-dihydroquinoline-3-carboxylate;
4-{[(8R,9S,10R,11R,13R,14R,16R,17S)-9-fluoro-11-hydroxy-10,13,16-trimethyl-17-{[(2-methylpropanoyl)oxy]acetyl}-3-oxo-6,7,8,9,10,11,12,13,14,15,16,17-dodecahydro-3H-cyclopenta[a]phenanthren-17-yl]oxy}-4-oxobutyl rel-1-cyclopropyl-6-fluoro-8-methoxy-7-(3-methylpiperazin-1-yl)-4-oxo-1,4-dihydroquinoline-3-carboxylate;
4-{2-[(8R,9S,10R,11R,13R,14R,16S,17S)-17-(butanoyloxy)-9-fluoro-11-hydroxy-10,13,16-trimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-[(4aS,7aS)-octahydro-6H-pyrrolo[3,4-b]pyridin-6-yl]-4-oxo-1,4-dihydroquinoline-3-carboxylate;
4-{[(8R,10S,11R,13R,14R,17S)-17-{[(2,2-dimethylpropanoyl)oxy]acetyl}-11-hydroxy-10,13-dimethyl-3-oxo-6,7,8,9,10,11,12,13,14,15,16,17-dodecahydro-3H-cyclopenta[a]phenanthren-17-yl]oxy}-4-oxobutyl rel-1-cyclopropyl-6-fluoro-8-methoxy-7-(3-methylpiperazin-1-yl)-4-oxo-1,4-dihydroquinoline-3-carboxylate;
4-({(8R,10S,11R,13R,14R,17S)-17-[(acetyloxy)acetyl]-11-hydroxy-10,13-dimethyl-3-oxo-6,7,8,9,10,11,12,13,14,15,16,17-dodecahydro-3H-cyclopenta[a]phenanthren-17-yl}oxy)-4-oxobutyl rel-1-cyclopropyl-6-fluoro-8-methoxy-7-(3-methylpiperazin-1-yl)-4-oxo-1,4-dihydroquinoline-3-carboxylate;
4-({(8R,9S,10R,11R,13R,14R,16S,17S)-17-[(acetyloxy)acetyl]-9-fluoro-11-hydroxy-10,13,16-trimethyl-3-oxo-6,7,8,9,10,11,12,13,14,15,16,17-dodecahydro-3H-cyclopenta[a]phenanthren-17-yl}oxy)-4-oxobutyl rel-1-cyclopropyl-6-fluoro-8-methoxy-7-(3-methylpiperazin-1-yl)-4-oxo-1,4-dihydroquinoline-3-carboxylate;
4-(2-{(8R,10S,11R,13R,14R,17S)-11-hydroxy-10,13-dimethyl-3-oxo-17-[(phenylcarbonyl)oxy]-6,7,8,9,10,11,12,13,14,15,16,17-dodecahydro-3H-cyclopenta[a]phenanthren-17-yl}-2-oxoethoxy)-4-oxobutyl rel-1-cyclopropyl-6-fluoro-8-methoxy-7-(3-methylpiperazin-1-yl)-4-oxo-1,4-dihydroquinoline-3-carboxylate;
4-{2-[(8R,10S,11R,13R,14R,17S)-17-(butanoyloxy)-11-hydroxy-10,13-dimethyl-3-oxo-6,7,8,9,10,11,12,13,14,15,16,17-dodecahydro-3H-cyclopenta[a]phenanthren-17-yl]-2-oxoethoxy}-4-oxobutyl rel-1-cyclopropyl-6-fluoro-8-methoxy-7-(3-methylpiperazin-1-yl)-4-oxo-1,4-dihydroquinoline-3-carboxylate;
4-({2-[(8R,9S,10R,11R,13R,14R,16R,17S)-17-(acetyloxy)-9-fluoro-11-hydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)benzyl rel-1-cyclopropyl-6-fluoro-8-methoxy-7-(3-methylpiperazin-1-yl)-4-oxo-1,4-dihydroquinoline-3-carboxylate;
4-({2-[(8R,9S,10R,11R,13R,14R,16R,17S)-17-(acetyloxy)-9-fluoro-11-hydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)benzyl rel-1-cyclopropyl-6-fluoro-8-methoxy-7-(3-methylpiperazin-1-yl)-4-oxo-1,4-dihydroquinoline-3-carboxylate;
4-({2-[(8R,9S,10R,11R,13R,14R,16R,17S)-17-(acetyloxy)-9-fluoro-11-hydroxy-10,13,16-trimethyl-3-oxo-6,7,8,9,10,11,12,13,14,15,16,17-dodecahydro-3H-cyclopenta[a]phenanthren-17-yl]-2-oxoethoxy}carbonyl)benzyl rel-1-cyclopropyl-6-fluoro-8-methoxy-7-(3-methylpiperazin-1-yl)-4-oxo-1,4-dihydroquinoline-3-carboxylate;
4-{[(8R,10S,11R,13R,14R,17S)-11-hydroxy-10,13-dimethyl-17-{[(2-methylpropanoyl)oxy]acetyl}-3-oxo-6,7,8,9,10,11,12,13,14,15,16,17-dodecahydro-3H-cyclopenta[a]phenanthren-17-yl]oxy}-4-oxobutyl rel-1-cyclopropyl-6-fluoro-8-methoxy-7-(3-methylpiperazin-1-yl)-4-oxo-1,4-dihydroquinoline-3-carboxylate;
(9R,10S,11S,13S,16S,17R)-17-[(acetyloxy)acetyl]-9-fluoro-17-hydroxy-10,13,16-trimethyl-3-oxo-6,7,8,9,10,11,12,13,14,15,16,17-dodecahydro-3H-cyclopenta[a]phenanthren-11-yl rel-3-({[1-cyclopropyl-6-fluoro-8-methoxy-7-(3-methylpiperazin-1-yl)-4-oxo-1,4-dihydroquinolin-3-yl]carbonyl}amino)propanoate;
4-({(8R,9S,10R,11R,13R,14R,16R,17S)-17-[(acetyloxy)acetyl]-9-fluoro-11-hydroxy-10,13,16-trimethyl-3-oxo-6,7,8,9,10,11,12,13,14,15,16,17-dodecahydro-3H-cyclopenta[a]phenanthren-17-yl}oxy)-4-oxobutyl rel-1-cyclopropyl-6-fluoro-8-methoxy-7-(3-methylpiperazin-1-yl)-4-oxo-1,4-dihydroquinoline-3-carboxylate;
(2R,3R)-2-[(dichloroacetyl)amino]-3-hydroxy-3-(4-nitrophenyl)propyl 2-[(8S,9R,10S,11S,13S,14S,16S,17R)-9-fluoro-11,17-dihydroxy-10,13,16-trimethyl-3-oxo-6,7,8,9,10,11,12,13,14,15,16,17-dodecahydro-3H-cyclopenta[a]phenanthren-17-yl]-2-oxoethyl rel-butanedioate; and
(2R,3R,4S,5R,6R)-6-((1S)-2-chloro-1-(1-methyl-4-propylpyrrolidine-2-carboxamido)propyl)-4,5-dihydroxy-2-(methylthio)tetrahydro-2H-pyran-3-yl (2-((10R,11S,13S,17R)-11,17-dihydroxy-10,13-dimethyl-3-oxo-6,7,8,9,10,11,12,13,14,15,16,17-dodecahydro-3H-cyclopenta[a]phenanthren-17-yl)-2-oxoethyl) succinate.

10. A method comprising administrating to an eye of a mammal a pharmaceutical composition comprising a therapeutically active amount of a hybrid drug comprising an antibiotic moiety and a steroid moiety, which are connected via two separate covalent bonds to a linker such that said covalent bonds degrade in vivo to yield the antibiotic and the steroid, wherein each bond is an ester bond or an amide bond, wherein said method is effective in the treatment of a bacterial infection or an inflammation affecting said eye.

11. The method according to claim 10, wherein the bacterial infection is selected from: conjunctivitis, keratitis, blepharitis, dacyrocystitis, hordeolum, corneal ulcers, anterior blepharitis, posterior blepharitis, endophthalmitis, meibomian gland dysfunction, dry eye disease (keratocojunctivitis sicca) ocular pain, ocular pain and inflammation post-ocular surgery, bacterial conjunctivitis, anterior uveitis, post-surgical inflammation, inflammatory conditions of the palpebral and bulbar conjunctiva, cornea, and anterior segment of the globe, such as allergic conjunctivitis, ocular rosacea, blepharitis, meibomian gland dysfunction, superficial punctate keratitis, herpes zoster keratitis, iritis, cyclitis, selected infective conjunctivitis, corneal injury from chemical radiation, or thermal burns, penetration of foreign bodies and allergy.

12. The method according to claim 10, wherein the mammal is a human.

13. A pharmaceutical composition comprising a hybrid drug comprising an antibiotic moiety and a steroid moiety, which are connected via two separate covalent bonds to a linker, wherein said covalent bonds degrade in vivo to yield the antibiotic moiety and the steroid moiety, and wherein each bond is an ester bond or an amide bond, and wherein said pharmaceutical composition is formulated for topical ophthalmic administration.
